SEQUENCE LISTING (73 sequences)

SEQ ID NO: 1 - DNA, 18 bp, Artificial Sequence
  The sequence has been designed and synthesized.
cctgctgcctagggaagc

SEQ ID NO: 2 - DNA, 23 bp, Artificial Sequence
  The sequence has been designed and synthesized.
cagatcagtttctatcagcctct

SEQ ID NO: 3 - DNA, 24 bp, Artificial Sequence
  The sequence has been designed and synthesized.
gcacaacttctgcactgaacggat

SEQ ID NO: 4 - DNA, 24 bp, Artificial Sequence
  The sequence has been designed and synthesized.
tcacacctgccagattgattccca

SEQ ID NO: 5 - DNA, 24 bp, Artificial Sequence
  The sequence has been designed and synthesized.
agtgacaacccagtcagttgcaga

SEQ ID NO: 6 - DNA, 24 bp, Artificial Sequence
  The sequence has been designed and synthesized.
agaagcgctcccaccaaagtctat

SEQ ID NO: 7 - DNA, 24 bp, Artificial Sequence
  The sequence has been designed and synthesized.
tgcagccctcctcacaagtgtaat

SEQ ID NO: 8 - DNA, 24 bp, Artificial Sequence
  The sequence has been designed and synthesized.
cgggctgcttacacacattcacaa

SEQ ID NO: 9 - DNA, 24 bp, Artificial Sequence
  The sequence has been designed and synthesized.
gtctctgccggctttacttcttgt

SEQ ID NO: 10 - DNA, 20 bp, Artificial Sequence
  The sequence has been designed and synthesized.
tgcgagaaagcggcacaggg

SEQ ID NO: 11 - DNA, 24 bp, Artificial Sequence
  The sequence has been designed and synthesized.
ttcaggtttggtttagcaaccgcc

SEQ ID NO: 12 - DNA, 24 bp, Artificial Sequence
  The sequence has been designed and synthesized.
tactgcttggatcagacacggctt

SEQ ID NO: 13 - DNA, 24 bp, Artificial Sequence
  The sequence has been designed and synthesized.
agccgtgtgctacgcatcaaattc

SEQ ID NO: 14 - DNA, 24 bp, Artificial Sequence
  The sequence has been designed and synthesized.
ttcctcttcaaaggcaggtctggt

SEQ ID NO: 15 - DNA, 24 bp, Artificial Sequence
  The sequence has been designed and synthesized.
acgacagcagctactacacggaat

SEQ ID NO: 16 - DNA, 24 bp, Artificial Sequence
  The sequence has been designed and synthesized.
tctccacaatgcttgagaggcagt

SEQ ID NO: 17 - DNA, 24 bp, Artificial Sequence
  The sequence has been designed and synthesized.
tgaaaccgcccaaatcctttccca

SEQ ID NO: 18 - DNA, 24 bp, Artificial Sequence
  The sequence has been designed and synthesized.
cgaagagcaacttggaaacagcca

SEQ ID NO: 19 - DNA, 24 bp, Artificial Sequence
  The sequence has been designed and synthesized.
gcagaatttcagaagatgcgccgt

SEQ ID NO: 20 - DNA, 24 bp, Artificial Sequence
  The sequence has been designed and synthesized.
tgactcgttgcaggttgtcgatct

SEQ ID NO: 21 - DNA, 24 bp, Artificial Sequence
  The sequence has been designed and synthesized.
tcaactttcgatggtagtcgccgt

SEQ ID NO: 22 - DNA, 24 bp, Artificial Sequence
  The sequence has been designed and synthesized.
tccttggatgtggtagccgtttct

SEQ ID NO: 23 - DNA, 24 bp, Artificial Sequence
  The sequence has been designed and synthesized.
acatagggcctgtctgcttcttgt

SEQ ID NO: 24 - DNA, 24 bp, Artificial Sequence
  The sequence has been designed and synthesized.
tgactgcggttggaaagtgtttgg

SEQ ID NO: 25 - DNA, 24 bp, Artificial Sequence
  The sequence has been designed and synthesized.
tcagaaccgtcgctacaagaccaa

SEQ ID NO: 26 - DNA, 24 bp, Artificial Sequence
  The sequence has been designed and synthesized.
cagcacctttacggccactttctt

SEQ ID NO: 27 - DNA, 24 bp, Artificial Sequence
  The sequence has been designed and synthesized.
aggtttcagatgcagtgaggagca

SEQ ID NO: 28 - DNA, 24 bp, Artificial Sequence
  The sequence has been designed and synthesized.
acatacagtccaggcagacccaaa

SEQ ID NO: 29 - DNA, 24 bp, Artificial Sequence
  The sequence has been designed and synthesized.
taccagcccacgtctattccacaa

SEQ ID NO: 30 - DNA, 24 bp, Artificial Sequence
  The sequence has been designed and synthesized.
tttggtgtacagtgctcggaggaa

SEQ ID NO: 31 - DNA, 24 bp, Artificial Sequence
  The sequence has been designed and synthesized.
ttcaaaggaggagactgttgggct

SEQ ID NO: 32 - DNA, 24 bp, Artificial Sequence
  The sequence has been designed and synthesized.
tgtggaggaggatgcatttggtct

SEQ ID NO: 33 - DNA, 24 bp, Artificial Sequence
  The sequence has been designed and synthesized.
aggcttacaagcaaatggcaaggg

SEQ ID NO: 34 - DNA, 24 bp, Artificial Sequence
  The sequence has been designed and synthesized.
accgcatggcatacttagcagaga

SEQ ID NO: 35 - DNA, 1288 bp, Gallus gallus
accttctcactgcgcgctggggccgttgacgtgcagcaggaacactataaaggcgagatggtgaaagtcggagtcaacggatttggccgtattggccgcctggtcaccagggctgccgtcctctctggcaaagtccaagtggtggccatcaatgatcccttcatcgatctgaactacatggtttacatgttcaaatatgattctacacacggacacttcaagggcactgtcaaggctgagaacgggaaacttgtgatcaatgggcacgccatcactatcttccaggagcgtgaccccagcaacatcaaatgggcagatgcaggtgctgagtatgttgtggagtccactggtgtcttcaccaccatggagaaggctggggctcatctgaagggtggtgctaagcgtgttatcatctcagctccctcagctgatgcccccatgtttgtgatgggtgtcaaccatgagaaatatgacaagtccctgaaaattgtcagcaatgcatcgtgcaccaccaactgcctggcacccttggccaaggtcatccatgacaactttggcattgtggagggtcttatgaccactgtccatgccatcacagccacacagaagacggtggatggcccctctgggaagctgtggagagatggcagaggtgctgcccagaacatcatcccagcgtccactggggctgctaaggctgtggggaaagtcatccctgagctgaatgggaagcttactggaatggctttccgtgtgccaacccccaatgtctctgttgttgacctgacctgccgtctggagaaaccagccaagtatgatgatatcaagagggtagtgaaggctgctgctgatgggcccctgaagggcatcctaggatacacagaggaccaggttgtctcctgtgacttcaatggtgacagccattcctccacctttgatgcgggtgctggcattgcactgaatgaccatttcgtcaagcttgtttcctggtatgacaatgagtttggatacagcaaccgtgttgtggacttgatggtccacatggcatccaaggagtgagccaggcacacagcccccctgctgcctagggaagcaggaccctttgttggagcccctgctcttcaccaccgctcagttctgcatcctgcagtgagaggccagttctgttcccttctgtctcccccactcctccaatttcttcctccacctgggggaggtgggagaggctgatagaaactgatctgtttgtgtaccaccttacatcaataaaagtgttcaccatctgaag

SEQ ID NO: 36 - protein, 333 aa, Gallus gallus
MVKVGVNGFGRIGRLVTRAAVLSGKVQVVAINDPFIDLNYMVYMFKYDSTHGHFKGTVKAENGKLVINGHAITIFQERDPSNIKWADAGAEYVVESTGVFTTMEKAGAHLKGGAKRVIISAPSADAPMFVMGVNHEKYDKSLKIVSNASCTTNCLAPLAKVIHDNFGIVEGLMTTVHAITATQKTVDGPSGKLWRDGRGAAQNIIPASTGAAKAVGKVIPELNGKLTGMAFRVPTPNVSVVDLTCRLEKPAKYDDIKRVVKAAADGPLKGILGYTEDQVVSCDFNGDSHSSTFDAGAGIALNDHFVKLVSWYDNEFGYSNRVVDLMVHMASKE

SEQ ID NO: 37 - DNA, 4837 bp, Gallus gallus
actgctcggcccgacggacggtgagagggcctccgccgcccgccatgcacggccgccgcccgccccgctccgccgctctcctcctcctcctcctccttctcacggccgccgcagccgcgcaggaccgcgacctccgacaacctggccccaagggacagaagggagaacccggagatattaaagatgttgtaggaccccgagggcctccaggaccacagggcccagcaggagagcagggacagcgaggggaccgtggcgagaagggggagaagggtgctcctggcccccgtgggagggatggagaacccggcacccctggaaacccaggcccccccggtccccccggacctcctggcccccccggacttggtggaaactttgcggcgcagatggcgggcggcttcgatgagaaggcgggtggagcgcagatgggtgtcatgcagggacccatgggccctatgggaccccgcggcccccctggccccactggcgcacctggtccccagggatttcaaggcaaccccggtgagcccggcgaacccggcgctgctggtccgatgggtccccggggacctccgggaccacctgggaaacccggtgacgatggtgagacaggcaaacccggcaaatctggtgaacgtggcccccccggcccccagggcgctcgtggcttccctgggactcctggtctccccggagtgaagggccaccgaggctaccccggtttggatggtgccaaaggagaggcgggggctcctggagccaagggtgaatctggttcaccgggtgagaacggctcccccggccccatgggaccccgtgggctgcccggagagcgaggacgtcccggcccctccggcgccgccggtgctcgtggcaatgacggtctccctggccctgctggaccccctggacccgtcggccctgccggagcccccggcttccccggagcccccggttcaaagggtgaagccggccccactggtgcacggggtcccgagggtgcccaaggaccccgcggcgaatccggcacccccggctctcccggccccgctggcgcacccggtaacccagggactgatggcatccccggtgccaagggctcggcgggtgccccgggcattgcaggcgctccaggattccccggcccacgcggcccccccggaccccaaggtgccaccggaccactgggacccaaaggacagacgggcgaacccggcatcgcaggcttcaagggcgagcaaggaccgaagggcgagacgggccccgcaggaccccaaggtgcccccgggccggctggtgaggaaggcaagagaggagctcgtggtgaacctggtgccgccggccctgtgggcccccccggagaaaggggcgctcctggcaaccgtggattccccgggcaggacgggctggccggacccaagggtgctccaggtgaacgcggccccgctggtctcgccggtcccaaaggtgccaccggtgaccccggacgtcccggagagcccgggctgcccggagcgaggggtctcaccggccgccccggcgatgcgggacctcaaggcaaagtcggcccaactggtgctcctggcgaggatggccgccccggcccccccggacctcagggtgctcgtgggcagcctggtgtgatgggtttccccggtcccaaaggcgctaatggtgagcctggaaaagctggagagaaaggactgcccggcgccccagggctgcggggtctgcctggcaaggatggggagacgggagctgccggcccccctggacccgctggtcctgtgggtgagagaggagagcaaggagcccccggtccttccggcttccagggactgcccggaccaccaggtccccctggggagagcggcaaacccggagaccagggtgttcctggagaagccggtgcccccggtcttgttggtcccagaggtgaacgtggattccccggtgaacgcggctctcccggtgcccaagggctgcagggtccccgtgggctccccggaacgcccggcactgacggacccaagggtgcaaccggtccagccggccccaacggtgcccagggtcccccagggctgcagggaatgcccggtgagagaggagcagctggcatcgctggcctcaagggtgaccggggagatgttggtgagaaaggacctgagggagctccaggcaaggatggcgcacgtggtctgacgggtcccattggtccccctggccctgctggccccaacggtgagaagggtgaatccggccctcctggtccatctggtgctgccggtgcccgtggtgcccccggtgagcgtggcgagcccggtgcccccggtcctgctggatttgctggccccccgggcgccgatggacaacccggtgccaaaggcgagcagggagagcccgggcagaagggtgacgcgggcgctcctggtccccaaggtccctccggcgctcctggcccccagggcccaaccggtgtcactggtcccaaaggagctcgtggggctcagggtccccctggagccacgggattccccggagctgccggccgtgtgggaccgcccggccctaatggtaacccaggcccccccggaccccctggctctgctggcaaggacggccccaagggtgttcgtggcgacgccggcccccccggccgtgcaggtgaccccggcctccaaggccccgccggcccccccggcgagaagggcgaacccggcgaggacggccccgcgggtcccgacggcccccccggccctcaaggcttggcaggacagcgtggtattgtgggtctcccaggacagcgtggtgagagaggcttccccggactgccggggccatcgggagaacctggaaagcaaggagcgcctggctctgcgggtgaccgaggtccccccggccccgtggggccccctgggctgacgggtcctgctggagaacccgggcgcgagggcaaccctggtgctgacggtctcccaggcagggatggcgcagctggcgtgaagggtgatcgtggtgagaccggccctgtgggtgcccccggtgctcctggagcccctggcgcccccggccctgttggtcccactggaaaacaaggagacagaggcgagacgggtgcacaagggcccatgggtccctctggtcccgctggagctcgaggaatgccgggtccccaaggacctcgtggtgacaaaggtgagacgggagaggctggagagagagggctgaagggccaccgtggcttcaccggtctgcagggtctgcccggaccacccggcccgtctggagaccaaggtgctgccggtcccgctggtccctccggtcccagaggtccccctggtcccgtcggcccctctggcaaagatggctctaacggcatgcccggccccatcggtcctcccggtccccgtggacggagtggtgaacccggccctgcgggtcctcctggaaaccccggtcctcccggtcctcctggcccccccggcaccggcatcgacatgtctgcttttgctggactgggtcagacggagaagggccccgaccccatccgctacatgagggcagacgaggcggccggagggctgcggcagcacgacgtggaggtggatgccaccctcaaatccctcaacaatcagattgagagcatccgcagccccgagggctccaagaagaaccctgccaggacctgccgcgacatcaaactctgccatcccgagtggaagagcggagattactggattgacccgaaccagggctgcaccttggacgccatcaaagtattctgcaacatggagacgggcgagacctgcgtctacccgacccccagcagcatccccaggaagaactggtggaccagcaagacgaaagacaagaagcacgtctggtttgcagagaccatcaacggcggtttccacttcagctacggcgatgagaacctgtcccccaacaccgccagcatccagatgaccttcctgcgcctcctgtccaccgagggctcccagaacgtcacctaccactgcaagaacagcatcgcctacatggacgaggagacgggcaacctgaagaaagccatcctcatccagggatccaacgacgtggagatcagagccgagggcaacagcaggttcacctacagcgtcttggaggacggctgcacgaaacacactggcaaatggggcaagacggtgatcgagtaccggttgcagaagacctcgcgcctgtccattgtagatactgcacctatggacattggcggagccgatcaggagtttggcgtggatattggcccagtctgcttcttgtaaaaagggttgttgttatttgtgtgtttgtttgttgtttggttgttgttttttgtttcttttttttttttttttagaaaagaaaggaatccagcccaatcccataaaagcaaaccagtcccacccccaggacccgcacgttcccagcacaacttctgcactgaacggatggcacgaccccgcgccccttcgggaccctccggcgccgtcaccgggcagactgcgaaatacaaccacgggcttatatttatttattgccttcctggaaggcctggtttcgtagggcgggtggaggtgggaatcaatctggcaggtgtgacggcccccctccccacaaagggatctggcaaacgcaggtatcgcgaatcccctcccctccccgtgtatcaccagcaggagtgctaatgtatcatacaacagaaatggtgctattcttgtaaaacaagtctgtattttttaacatcagttgatataaaaacaacaaaaaaaaaaacttttggtggaaagtaaaaaaaacaaaaaaaaaaaaaaaa

SEQ ID NO: 38 - protein, 1420 aa, Gallus gallus
MHGRRPPRSAALLLLLLLLTAAAAAQDRDLRQPGPKGQKGEPGDIKDVVGPRGPPGPQGPAGEQGQRGDRGEKGEKGAPGPRGRDGEPGTPGNPGPPGPPGPPGPPGLGGNFAAQMAGGFDEKAGGAQMGVMQGPMGPMGPRGPPGPTGAPGPQGFQGNPGEPGEPGAAGPMGPRGPPGPPGKPGDDGETGKPGKSGERGPPGPQGARGFPGTPGLPGVKGHRGYPGLDGAKGEAGAPGAKGESGSPGENGSPGPMGPRGLPGERGRPGPSGAAGARGNDGLPGPAGPPGPVGPAGAPGFPGAPGSKGEAGPTGARGPEGAQGPRGESGTPGSPGPAGAPGNPGTDGIPGAKGSAGAPGIAGAPGFPGPRGPPGPQGATGPLGPKGQTGEPGIAGFKGEQGPKGETGPAGPQGAPGPAGEEGKRGARGEPGAAGPVGPPGERGAPGNRGFPGQDGLAGPKGAPGERGPAGLAGPKGATGDPGRPGEPGLPGARGLTGRPGDAGPQGKVGPTGAPGEDGRPGPPGPQGARGQPGVMGFPGPKGANGEPGKAGEKGLPGAPGLRGLPGKDGETGAAGPPGPAGPVGERGEQGAPGPSGFQGLPGPPGPPGESGKPGDQGVPGEAGAPGLVGPRGERGFPGERGSPGAQGLQGPRGLPGTPGTDGPKGATGPAGPNGAQGPPGLQGMPGERGAAGIAGLKGDRGDVGEKGPEGAPGKDGARGLTGPIGPPGPAGPNGEKGESGPPGPSGAAGARGAPGERGEPGAPGPAGFAGPPGADGQPGAKGEQGEPGQKGDAGAPGPQGPSGAPGPQGPTGVTGPKGARGAQGPPGATGFPGAAGRVGPPGPNGNPGPPGPPGSAGKDGPKGVRGDAGPPGRAGDPGLQGPAGPPGEKGEPGEDGPAGPDGPPGPQGLAGQRGIVGLPGQRGERGFPGLPGPSGEPGKQGAPGSAGDRGPPGPVGPPGLTGPAGEPGREGNPGADGLPGRDGAAGVKGDRGETGPVGAPGAPGAPGAPGPVGPTGKQGDRGETGAQGPMGPSGPAGARGMPGPQGPRGDKGETGEAGERGLKGHRGFTGLQGLPGPPGPSGDQGAAGPAGPSGPRGPPGPVGPSGKDGSNGMPGPIGPPGPRGRSGEPGPAGPPGNPGPPGPPGPPGTGIDMSAFAGLGQTEKGPDPIRYMRADEAAGGLRQHDVEVDATLKSLNNQIESIRSPEGSKKNPARTCRDIKLCHPEWKSGDYWIDPNQGCTLDAIKVFCNMETGETCVYPTPSSIPRKNWWTSKTKDKKHVWFAETINGGFHFSYGDENLSPNTASIQMTFLRLLSTEGSQNVTYHCKNSIAYMDEETGNLKKAILIQGSNDVEIRAEGNSRFTYSVLEDGCTKHTGKWGKTVIEYRLQKTSRLSIVDTAPMDIGGADQEFGVDIGPVCFL

SEQ ID NO: 39 - DNA, 6470 bp, Gallus gallus
atgaccactctactactagtgtttgtgtgtttgcaagccatcaccacagctgcctccgcagagctctcagacagtagcgatggcctggaagtgaagatacctgagcagtctcccctgcgtgttgtcctgggaagctccctgaacatcccctgctatttcaacatcccagaggaagaggacaccaatgctctgctgaccccccggatcaaatggagcaagctttccaatggaacagagattgtcttactagtggccaccggtgggaagatccggctcaatgcagagtacagagaggtgatctccttgcccaattaccctgccatccccactgatgccaccttggaaatcaaggcgctgagatccaaccacactgggatttatcgctgtgaagtgatgtatgggattgaggacagacaagacaccatagaggtcctggtgaaaggcgtcgtgttccactacagagcaatctccacaaggtacactttgaacttcgagagggcaaagcaggcctgtatccagaacagtgctgtcattgccacccctgagcagctgcaggctgcctacgaggatgggtacgagcagtgcgatgccggctggctggctgatcagactgtcaggtaccccatccatctgccccgggagcgctgctacggtgacaaggatgagtttccaggagtgagaacctacggtgtccgtgagacagatgaaacctatgatgtttactgctatgcagagcaaatgcaaggcaaagtcttctacgccacctcccccgagaagttcaccttccaggaagcttttgacaaatgccacagcttgggagcccgcctggccaccacgggcgagctgtacttggcctggaaggatggcatggacatgtgcagtgcgggctggctggctgaccgcagcgttcgctaccccatttccagagcacggcccaattgtggagggaacctggtgggcgtgcggaccgtttacctgaaccctgccaaccagacagggtaccctcaccccagctcacgctacgatgccatctgttacagtggtgatgacttcgaggctctggtcccagggctgttcactgacgaggttgggactgagctgggcagtgcttttaccatacagaccgtcacacagactgaggtggagctgcccctgccacgcaatgtcacagaagaggaggcccgtggcagcatcgctaccctggagcccatggagatcacagccaccgccactgagctgtatgaggccttcaccgtcctgcctgacctctttgccaccagtgtcacagtagagacagcttccccaagggaagagaacgtgaccagagaggagatcacagggatatgggctgtgcctgaagaggtcactacatcggtctcaggcactgctttcaccaccggaatggcagaggtgagctcagtggaagaggccatagcagtgactgccacaccaggactggagtctgcctctccattcaccatagaagatcatctcgtgcaagtgacagcagcccctgatgttgccctcctccccaggcagcccatttcccccactggtgtggtgttccactaccgtgctgccaccagcagatacgccttctccttcatccaagcccagcaggcctgcctggagaacaatgccgttattgccactcccgagcagctccaggctgcctacgaggctggctttgatcagtgcgatgccggctggctgcgggaccagacggtcaggtatcccattgtgaatccccgcagcaactgtgtaggagacaaagagagctccccaggtgtgcggtcatacggcatgcgcccggcctcagagacctacgatgtgtactgttacatcgacaggctaaagggtgaggtgttttttgccacccagccagagcagttcaccttccaagaagcccagctgtactgtgaaagccaaaatgccacgctggcctctgctgggcaactccacgctgcctggaagcagggcctggataggtgctaccctgggtggctggccgacggcagcttgcggtaccccattgtgagcccccgacctgcctgcgggggggatgcacctggtgtgaggaccatctaccagcaccacaaccagacgggctttcctgaccctctgtcacggcatcacgctttctgcttcagagctctgccatccgtagtggaggagggggtgacctcactctttgaagaagaggtgatggtaacccaactgatccctggagtggaaggaataccttctggggaggaaacaactgtggagacagagctttcctctgagcctgagaatcagacagcccagggaacggaggtcttcccaactgacgtatcactgctctcagcgagaccatctgcttttcctccagctactgtaataccagaggaaacaagtaccaatgcttccatccccgaagtgtccggagagttccctgagtctggagagcacccaaccagtggcgaaccttcagcatctggagccccagacacgagtggagaaccaacttccgtaggttttgaactgagtggagagcaatccgggattggagaaagtggattaccatctgtagacctgcagagcagtggctttgtacctggagaaagtggccttccctcaggggatgtgagcgggttgccttctggcattgttgatatcagcggcttgccttctgcagaggaagaggtaacggtgtctgtttcgaggataccagaagttagtggaatgccatctggagctgaaagcagtggtctgcattctggatttagtggagaaatctctggcactgagcttatcagtggcctgccatctggagaggaaagtgggctcgcctctggttttcccaccatctccctcgtggattccactttggtggaggttgtaacggcagcaccgggacggcaagaggagggaaaaggatcaattggagtcagtggtgaagaagagctgtcagggtttccgtctgcagagtgggacagcagtggggccagagggctgccctcgggagctgaaaccagtggggagcaatccggggtgcccgagctcagtggggagcattccggggtgcctgggctcagtggagagccttttgaggtgcctgagctcagcggggagcattccggggtgactgagctcagtggggagcattctgggcttcctgagctcagtggagagccttttggggtgcctgagctcagcgggtttccctccggactggacatcagtggggaaccatctggagcacccgaggtgagcggcccggtggacgtgagcggccttacctctggtgttgatggaagtggtgaggtctcaggcgttacctttataagcaccagcctgcaagaagtgacaacccagtcagttgcagaagcagaagcaaaagaaattctagagatcagtggactgccttcaggagagacatcaggcatggtgtctgggagcttagatgtcagcggtcagccttcgggacacatagactttggtgggagcgcttctggagtgctggagatgagcggatttccaagcggagcggttgagagcagtggagaagcctctggggttgaagtcaccagtgtcctcgcgtctggagaggaaagtgggctcacctcaggctttcccaccgtgtctctcgtggataccacgttggtagaagttgtaacgcagacatcagttgctcaagaggtgggagaaggaccatctgggatgatagaaatcagtggatttctttctggagacagaggagtatctggagaagggtctggagctgtgcagtctagtgggcttccttcaggaacaggagacttcagcggagagccatccgggatcccgtatttcagcggagacatttctggagccacagatctaagcggacaaccttccgcagtgactgatattagtggggaggactcgggacttccagaagtcactttagtcacatccgatttagtagaagttgtgacaaggccaacagtttcacaggagctgggtggggaaacagctgtgacgtttccctatgtctttgggccaagtggtgagggctctgcatctggagacctgagtgggggagcatctgcagaaggtggtatagaaacatcagcagcttatgaaatcagtggtgagagctctgccttccctgaaacgagtatagaaacatccacagatcaagaaatcagtggggaagcatctgcataccctgagattagcgtagaaacatccacgcatctggaaaccagtggggaaacatctgcataccctgagattagcacagagacgtccaccattcaggaagttagtggggaaacgtctgcctttcctgaaattagcacagaaacatccacaatacaagagatcagtggggaaacatccgcatttcctgaaattagaatagaaacatccacctttcaagaaatcagtggggaaacgtccgcatttcctgaaattagaatagaaacatccacgagtcaagaagcccgcggcgaaacgtctgcgtttcccgagattaccatagaagcctccacagtccatgaaaccagtggagaaacatctgcctttcccgaaattagcatagaaacatccacagtccatgaaatcagtggggaaagttctgcctttcctgaaattagaatagaaacatccacgagtcaagaagcccggggtgaaacatctgcgttccctgagattaccatagaagcctccaccattcaagagatcagtggagaaacatctgcatttcctgaaattagcatagcaacttccaccgtccgtgaaatcagcggcgaaacgtctgcctttcctgaaattagaatagaaacatccacgagtcaagaagcccggggtgaaacatctgcattgcctgagattaccgtagagacatccactgtccatgaaaccagtggggaagcgtctgcctttcctgaaattagcatagaaacttccacaagacaagaagccaggggtgaagcatccgcctaccctgaggttagcatagaggcatccacaactcaagaagtaagtggggaaagttctgccttccctgaaattagcgtagagacatccacaagtcaggaagcccgcggtgaaacatctgcctttcctgagattggcatagagacatccacagcccacgaaggcagtggggaaactcctgggctgcctgctgttagcactgacactgctgccacgtctctggccagtggtgagccctccggtgctcctgagaaggaaactcccgacacaacatcacatctgatcacgggcgtttcaggggaaacctctgtcccagatgctgtaatcagtaccagtgctccagatgttgaactagcgcagggacccagaaacactgaagagactcagcttgaaatagagccctccactcctgcggcatctggacaagagacagaaacagctgctgtcctcgacaatccccatctgccagccactgctactgctgccctgcatccagcctcccaagaagcagtagatgcactgggacccacgacagaagacactgatgagtgccactcaagcccctgtctgaatggagctacctgcgttgatggcatcgactctttcaaatgcttatgccttcccagctacggaggggacctgtgtgagatcgacctggcaaactgtgaggaaggctggatcaagttccagggccactgctacaggcactttgaagagagggagacgtggatggacgcagagtccaggtgcagagaacatcaagcccacctgagcagcatcattactccagaggaacaagaatttgtgaacagccatgcacaagactatcagtggatcggcctcagtgacagagccgtggagaatgatttccgctggtctgatggacactcgctgcaatttgagaactggcggcccaaccaacctgacaacttcttctccgcgggcgaggactgcgttgtgatgatctggcatgagcaaggcgaatggaacgacgtcccctgcaactatcacctgcctttcacgtgcaagaagggaacagttgcctgtggggacccacctgtagtggagaacgcgcggacctttggccggaagaaggaccgctatgagatcaactccctggtgcggtaccagtgtgaccacggctacatccagcgccacgtgcccaccatccgctgccaacccaacgggcactgggaggaaccgcggatatcctgcacaaacccctccagctaccaacgccggctatacaagaggagcccccggagccggttgagacccggtgtcgtgcacagacccacccattagagcggggcgaggggtgagcagagagagaaaagagagatttttacggagcacttctattaacagagtcaatttcttcttcttgttgttgcttttttgtcatataaggaaaaaaaaaaaaaagaattatattacagacaaacccacctttgtgtatacatatataaaaataataataataataatgttttccaagcaccaaa

SEQ ID NO: 40 - protein, 2089 aa, Gallus gallus
MTTLLLVFVCLQAITTAASAELSDSSDGLEVKIPEQSPLRVVLGSSLNIPCYFNIPEEEDTNALLTPRIKWSKLSNGTEIVLLVATGGKIRLNAEYREVISLPNYPAIPTDATLEIKALRSNHTGIYRCEVMYGIEDRQDTIEVLVKGVVFHYRAISTRYTLNFERAKQACIQNSAVIATPEQLQAAYEDGYEQCDAGWLADQTVRYPIHLPRERCYGDKDEFPGVRTYGVRETDETYDVYCYAEQMQGKVFYATSPEKFTFQEAFDKCHSLGARLATTGELYLAWKDGMDMCSAGWLADRSVRYPISRARPNCGGNLVGVRTVYLNPANQTGYPHPSSRYDAICYSGDDFEALVPGLFTDEVGTELGSAFTIQTVTQTEVELPLPRNVTEEEARGSIATLEPMEITATATELYEAFTVLPDLFATSVTVETASPREENVTREEITGIWAVPEEVTTSVSGTAFTTGMAEVSSVEEAIAVTATPGLESASPFTIEDHLVQVTAAPDVALLPRQPISPTGVVFHYRAATSRYAFSFIQAQQACLENNAVIATPEQLQAAYEAGFDQCDAGWLRDQTVRYPIVNPRSNCVGDKESSPGVRSYGMRPASETYDVYCYIDRLKGEVFFATQPEQFTFQEAQLYCESQNATLASAGQLHAAWKQGLDRCYPGWLADGSLRYPIVSPRPACGGDAPGVRTIYQHHNQTGFPDPLSRHHAFCFRALPSVVEEGVTSLFEEEVMVTQLIPGVEGIPSGEETTVETELSSEPENQTAQGTEVFPTDVSLLSARPSAFPPATVIPEETSTNASIPEVSGEFPESGEHPTSGEPSASGAPDTSGEPTSVGFELSGEQSGIGESGLPSVDLQSSGFVPGESGLPSGDVSGLPSGIVDISGLPSAEEEVTVSVSRIPEVSGMPSGAESSGLHSGFSGEISGTELISGLPSGEESGLASGFPTISLVDSTLVEVVTAAPGRQEEGKGSIGVSGEEELSGFPSAEWDSSGARGLPSGAETSGEQSGVPELSGEHSGVPGLSGEPFEVPELSGEHSGVTELSGEHSGLPELSGEPFGVPELSGFPSGLDISGEPSGAPEVSGPVDVSGLTSGVDGSGEVSGVTFISTSLQEVTTQSVAEAEAKEILEISGLPSGETSGMVSGSLDVSGQPSGHIDFGGSASGVLEMSGFPSGAVESSGEASGVEVTSVLASGEESGLTSGFPTVSLVDTTLVEVVTQTSVAQEVGEGPSGMIEISGFLSGDRGVSGEGSGAVQSSGLPSGTGDFSGEPSGIPYFSGDISGATDLSGQPSAVTDISGEDSGLPEVTLVTSDLVEVVTRPTVSQELGGETAVTFPYVFGPSGEGSASGDLSGGASAEGGIETSAAYEISGESSAFPETSIETSTDQEISGEASAYPEISVETSTHLETSGETSAYPEISTETSTIQEVSGETSAFPEISTETSTIQEISGETSAFPEIRIETSTFQEISGETSAFPEIRIETSTSQEARGETSAFPEITIEASTVHETSGETSAFPEISIETSTVHEISGESSAFPEIRIETSTSQEARGETSAFPEITIEASTIQEISGETSAFPEISIATSTVREISGETSAFPEIRIETSTSQEARGETSALPEITVETSTVHETSGEASAFPEISIETSTRQEARGEASAYPEVSIEASTTQEVSGESSAFPEISVETSTSQEARGETSAFPEIGIETSTAHEGSGETPGLPAVSTDTAATSLASGEPSGAPEKETPDTTSHLITGVSGETSVPDAVISTSAPDVELAQGPRNTEETQLEIEPSTPAASGQETETAAVLDNPHLPATATAALHPASQEAVDALGPTTEDTDECHSSPCLNGATCVDGIDSFKCLCLPSYGGDLCEIDLANCEEGWIKFQGHCYRHFEERETWMDAESRCREHQAHLSSIITPEEQEFVNSHAQDYQWIGLSDRAVENDFRWSDGHSLQFENWRPNQPDNFFSAGEDCVVMIWHEQGEWNDVPCNYHLPFTCKKGTVACGDPPVVENARTFGRKKDRYEINSLVRYQCDHGYIQRHVPTIRCQPNGHWEEPRISCTNPSSYQRRLYKRSPRSRLRPGVVHRPTH

SEQ ID NO: 41 - DNA, 1842 bp, Gallus gallus
cggcggcggcggcggcggcgcggccggccggagcgggcgatgcgggcgccgagctgaagttgcggcgcttgtcgctgctctccgcccggggccgccgggccatggccgtccgcggcggcggcggcggcggcggcaccgccctgacgcctttctccatccaggccatcctcaacaagaaggaggagcgcgcccgcggctggcggctgtgcgctgcccgcgccgaggccccgccgccgcccgccccgcgcccgccgcccgcctgggactcggactcggcgctgagcgaggagcccgagagcgagcggcgctccgaggaggagagcgccgggggcagcgcccgcccccccgaggcggcgggcaggggggcggccggcggcggaggcgcggccctcggaggcggcggcggacgggaccgggaccgggacggagcgcgcagcgacagcgaagcgtcggccgccgcctcaggtcgcggcccggccgaggaggaggaggcggcgggcgggaggctgctggcggccgaggaggaggcggcggcgccgaagccgcgcaagaagcgctcccgcgccgccttctcccacgcgcaggtgttcgagctggagcggcgcttcaaccaccagcgctacctgtcggggcccgagcgcgccgacctggccgcctccctcaagctgaccgagacgcaggtgaagatctggttccagaaccgccgctacaaaaccaagcggcggcagatggcagccgacctgctggccgccgcccccgccgccaagaaggtggccgtcaaggtgctggtgcgcgacgaccagagacagtaccagcccggcgaggtgctgcggccgccctcgctgctcgccctgcagccctcctactactacccctactactgcctgcccggctgggccctgtcctgcgccgcggccgccggcacgcagtgagcgccccgacggcaccgccccgacggcagcgccgcggcgccccggacagtatttattgctattttattgttatttttattattaatattattcggacggtcgctcggcctctccctatcgccccggcggccgaggactcgcggccgggcaccgcctctccacgggatccacgctgcggtgcggcggccgcggaccgtctgttggtttgtctgctccgtcccgcgtcggatcggcggagactgacgtgggagacgcaaagagagaggggggaaagaaaacacccaaagcgccgcggaaccgagcgcagctccgaggagacgagaacggatcccggggggctctgagagcgccgtgagcggggctgaatgtacggagcctctcccacccccggccgtgtcagcccactgcgaaggataaccgtgtcccgtagggtgagacccgccccagcgctccaggcttcttcttcttcttcttctgttttatttcctgagatctttccgtgttcggggagggaggattctggatttttcgcactttccggttgtattaaagttgttgttatttttatattcaatgtgaatatttctaggcaggctttaaaaaaaaaacagaaaagaaaactcgatgtaacgggaaatgttaatgccatcagtgccttttgtgcagtcgttctgcagccctcctcacaagtgtaattacggtctctttccctcgaggtaacccatcgctttgtttctttctttctttctttttctccctttaatggcactgtgcactcaacgacgctatcgactccaaatggattgtgaatgtgtgtaagcagcccgctgtactttttttttttttttttttttaatttataaacgttacgttcccaaaaaaaaaaaaaaaaaaaaaaaaaaaaaaaaaa

SEQ ID NO: 42 - protein, 276 aa, Gallus gallus
MAVRGGGGGGGTALTPFSIQAILNKKEERARGWRLCAARAEAPPPPAPRPPPAWDSDSALSEEPESERRSEEESAGGSARPPEAAGRGAAGGGGAALGGGGGRDRDRDGARSDSEASAAASGRGPAEEEEAAGGRLLAAEEEAAAPKPRKKRSRAAFSHAQVFELERRFNHQRYLSGPERADLAASLKLTETQVKIWFQNRRYKTKRRQMAADLLAAAPAAKKVAVKVLVRDDQRQYQPGEVLRPPSLLALQPSYYYPYYCLPGWALSCAAAAGTQ

SEQ ID NO: 43 - DNA, 1640 bp, Gallus gallus
ctccgtctctgccggctttacttcttgtttttaacccttccccgcccctcagccgcccggttgttttttttctctccgttttctcctcccctgatccccctgtgccgctttctcgcatgaatctcctagaccccttcatgaaaatgacagaagaacaggacaaatgcatctccgacgcccccagccccaccatgtcggatgactccgccgggtccccctgcccctccggatccggctcggacacggagaacacccgacctcaagagaacaccttccccaaaggcgacccggacctgaagaaggagagcgacgaggacaaattccccgtgtgcatccgcgaggccgtgagccaggtgctcaagggctacgactggaccctggtgcccatgcccgtgcgggtgaacggatccagcaagaacaaaccccacgtgaagcgccccatgaacgccttcatggtgtgggcccaggcggctcgaaggaagctggctgaccagtacccgcatctgcacaacgccgagctcagcaagacgctgggcaagctgtggaggctgctgaatgagagcgagaagcgtcccttcgtggaggaggccgagcggctgcgggtgcagcacaagaaggaccaccccgactacaagtaccaaccacgcaggaggaagtcggtgaagaacgggcagtcggagcaggaggagggctccgagcagacccacatctcccccaacgccatcttcaaggcgctgcaggcggactccccgcagtcatcctccagcatcagcgaggtgcactcccccggggagcactcagggcagtcgcagggcccccccacgccccccaccacccccaaaacggacgctcagcagccgggcaagcaggacctgaagcgcgagggccgccctttggcggaaggcggccgccaacctccccacatcgatttccgagacgtggacatcggcgagctcagcagcgacgtcatctccaacatcgaaaccttcgacgtcaacgagttcgaccaatacctgccccccaacggccacccgggggtcccggccacccacggccaggtcaccacctacagcggtacctacggcatcagcagctcggccagctctccggcgggcgccgggcacgcctggatggccaagcagcagccgcagcccccacagcccccagcacagcccccggcacagcacacactgccagcactgagcggtgagcagggtccggcacagcagcggccgcacatcaaaacagagcagctgagccccagccactacagcgagcagcagcagcactccccgcagcagcagcagcagcagcaacagcagctgggctacggctccttcaacctgcagcactacggctcctcgtacccccccatcacccgctcgcagtacgattacaccgagcaccagaactccggctcctactacagccacgccgccgggcagagcggaggcctctactccaccttcacctacatgaaccccacgcagcgccccatgtacacccccatcgcagacacgtcgggggtgccctctatcccgcagacccacagcccgcagcactgggaacagcccgtctacacgcagctcacccggccttaaagccacgtgaaggcaaataaataaataaacaaaccaatc

SEQ ID NO: 44 - protein, 494 aa, Gallus gallus
MNLLDPFMKMTEEQDKCISDAPSPTMSDDSAGSPCPSGSGSDTENTRPQENTFPKGDPDLKKESDEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQSEQEEGSEQTHISPNAIFKALQADSPQSSSSISEVHSPGEHSGQSQGPPTPPTTPKTDAQQPGKQDLKREGRPLAEGGRQPPHIDFRDVDIGELSSDVISNIETFDVNEFDQYLPPNGHPGVPATHGQVTTYSGTYGISSSASSPAGAGHAWMAKQQPQPPQPPAQPPAQHTLPALSGEQGPAQQRPHIKTEQLSPSHYSEQQQHSPQQQQQQQQQLGYGSFNLQHYGSSYPPITRSQYDYTEHQNSGSYYSHAAGQSGGLYSTFTYMNPTQRPMYTPIADTSGVPSIPQTHSPQHWEQPVYTQLTRP

SEQ ID NO: 45 - DNA, 1566 bp, Gallus gallus
gagcgcgccgcgccgccgcgatgaccacgctggccggggccgtgcccaggatgatgcggcccggagccgggcagagctacccgagaggaggcttcccgttggaagtctctacccctctcggccagggcagagtcaatcagctcggaggagtgttcatcaacgggcggccgctgcccaaccatatccgccacaagatcgtggagatggcgcaccacggcatccggccctgtgtcatctcccggcagctgcgcgtgtcccacggctgcgtctccaagatcctctgccgctaccaggagacgggatccatccgcccgggggccatcggcggcagcaaacccaagcaggtgacgacgccggacgtggagaagaaaatcgaggagtacaagcgggagaacgcggggatgttcagctgggagatccgcgacaggctgctcaaggacggcgtctgcgaccgcaacaccgtgccctcagtgagctccatcagccgcatcctgcggagcaagttcgggaaaggcgaggaggaggaggccgagctggagaggaaggaggcggaggaaggagacaagaaggcgaagcacagcatcgacggcatcctcagcgagagagcatcagcagcccagtctgatgaaggctctgatatcgattctgaaccagacttacccttaaaaagaaagcagcgtcgcagcaggacaaccttcacagcagagcaactggaagagctggaaagagcttttgagaggacacactaccctgacatttatactcgggaagaactcgcacaaagagccaaactcacggaagctcgagttcaggtttggtttagcaaccgccgtgctagatggaggaagcaggcaggagccaaccaactgatggcttttaaccacctgatcccaggagggtttccacccagcgccatgccgactctgccgacataccagctctctgagccatcctatcagcccacctccataccgcaagccgtgtctgatccaagcagtacagtccatagacctcagcctctccctccaagcactgtacaccaaagcagcctcccttcaaacccagagagcagctctgcctattgcctacccagcaccaggcatggattttccagctatacagacagctttgtgcctccgtccgggccctcaaatcccatgaaccctgccattggcaatggcctttcacctcaggtaatgggactcttgactaaccatggtggtgtgcctcaccagccccaaactgattatgccctgtcccctttgactggaggtctggagcccaccaccactgtctcagccagctgcagtcagcggctagatcacatgaagagtttagacagcctgcctacatcacagtcctactgcccaccgacctatagcaccaccggctacagcatggaccctgtcaccggctaccagtatggacagtatggacaaagtgcctttcattatctgaagccagatatcgcataaatgaactgtccacttcaagttaaagctggtgttattttccggtctcgctccagagcttggataccaggggatcttctcaacacactgcagt

SEQ ID NO: 46 - protein, 484 aa, Gallus gallus
MTTLAGAVPRMMRPGAGQSYPRGGFPLEVSTPLGQGRVNQLGGVFINGRPLPNHIRHKIVEMAHHGIRPCVISRQLRVSHGCVSKILCRYQETGSIRPGAIGGSKPKQVTTPDVEKKIEEYKRENAGMFSWEIRDRLLKDGVCDRNTVPSVSSISRILRSKFGKGEEEEAELERKEAEEGDKKAKHSIDGILSERASAAQSDEGSDIDSEPDLPLKRKQRRSRTTFTAEQLEELERAFERTHYPDIYTREELAQRAKLTEARVQVWFSNRRARWRKQAGANQLMAFNHLIPGGFPPSAMPTLPTYQLSEPSYQPTSIPQAVSDPSSTVHRPQPLPPSTVHQSSLPSNPESSSAYCLPSTRHGFSSYTDSFVPPSGPSNPMNPAIGNGLSPQVMGLLTNHGGVPHQPQTDYALSPLTGGLEPTTTVSASCSQRLDHMKSLDSLPTSQSYCPPTYSTTGYSMDPVTGYQYGQYGQSAFHYLKPDIA

SEQ ID NO: 47 - DNA, 2132 bp, Gallus gallus
ccggagcgcccgctcaggccggttgtgacatagcccgaaaactttccgagggatttctgccgcgggctgggagaccgccgaaagccttcagctaccggacacgagagagagcccccacgtctctcggctctcccggggttcgggggtgacccgctccgcacggagccgctccgccgccccccgctatggcagcgctccccgggacggtaccgcggatgatgcgcccggcgccggggcagaactacccgcgcaccggcttccctttggaagtgtccaccccgctgggccagggccgggtgaaccaactcggaggggttttcatcaacgggcgcccactgcccaaccacatccgccataagatcgtggagatggctcaccacggcatccggccctgcgtgatctccaggcagctgagggtctcccacggctgcgtttccaaaatcctctgcaggtaccaagagacgggctccatccggcccggggccatcgggggcagcaagcccaggcaggttgcgactcccgacgtggagaagaaaatcgaggaatacaagagggagaaccctgggatgttcagctgggagatccgggacaggctgctgaaggacggacactgcgaccgcagcactgtgccctcagtgagttcgattagccgtgtgctacgcatcaaattcgggaagaaagaggaagaggaggactgcgacaagaaggaggaagacggggagaagaaggccaagcacagcatagatggcatcctgggcgacaaagggaacaggctggatgaaggctccgatgtcgaatcagaaccagacctgcctttgaagaggaagcagcgccgcagccggaccactttcactgccgagcagctggaggagctggagaaggcctttgagaggacccactacccggacatctacaccagggaggagctggcacagagaaccaagctcaccgaggcccgtgttcaggtgtggttcagcaaccgacgagcaagatggcgcaagcaggcgggtgcaaaccaactcgcagcattcaaccatctgctgccagggggattcccacccacgggaatgccaactctgcccccgtaccagctgccagactccacctacccaaccaccaccatttcccaagatggaggcagcaccgtgcacagaccccagcccttgccaccatccaccatgcaccagggagggctcgctgccgccgctgcagccgactccagctctgcctatggggcccgacacagcttctccagctactcagacagcttcatgaatgctgcagctcctgccaaccacatgaatcctgttagcaatggcctctctccgcagaagcagggtgcccaaaacaagatgcagtgctccaggtggaacctcaccatagccttgaacaatcaggtgatgagcatcctgagcaaccccagcggggttcctccgcagccccaggctgacttctccatctctcctcttcacggtggcctggacaccaccaactccatctctgccagctgcagccagcggagtgactccatcaagtccgtggacagcctcccgacctcgcagtcctactgtcctcccacctacagcaccaccagttacagcgtggacccggtggctggctaccagtatgggcagtatggacaaactgctgttgattatttgaccaagaacgtgagcctgtccacgcagcgcaggatgaagctgggagaacattcggccgttctggggctcctaccagtagagacaggccaagcttactgaagaggtccgactgtgccaacaacccactccagcaacttggtaccactggagaaacactaccagcttccaggatctgctgtacccaagaccctactccttcccaactcccttccacctcgcatcaacttgtggaaggaagacgggaaagatggggagggctctccaagaatccagctcctcccagggcgctcagaggcaagcctggcctcattgacctgaattctgtggccagagacaataggttgggtacatttattaacccgagttcatgcctcctctactggccatgtgtctgcccatgctaaagaccttccattgatctagtagttggcatagtcaaagccaaattgatctttttttcttttcctttgt

SEQ ID NO: 48 - protein, 524 aa, Gallus gallus
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEEEDCDKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAADSSSAYGARHSFSSYSDSFMNAAAPANHMNPVSNGLSPQKQGAQNKMQCSRWNLTIALNNQVMSILSNPSGVPPQPQADFSISPLHGGLDTTNSISASCSQRSDSIKSVDSLPTSQSYCPPTYSTTSYSVDPVAGYQYGQYGQTAVDYLTKNVSLSTQRRMKLGEHSAVLGLLPVETGQAY

SEQ ID NO: 49 - DNA, 1518 bp, Gallus gallus
gtcggcggtggtggcagcagcaacccgcgccggtggcctcgcctgggacagggtgcgagggccccgctccgtgcccacctcgcacagccaccctctggaccccccgtgcccccgaccgccatctcaccccactccgacgttcccagtcgcccccatggacttactgggccccatggaaatgacggagggctccctctgctccttcacggccgccgatgacttctatgacgacccgtgcttcaacacgtcggacatgcacttcttcgaggacctggacccccggctggtgcacgtgggcgggctgctgaaagccgaggagcacccgcacacacgggcaccaccacgggaacccacagaggaggagcacgtgcgggcgcccagtgggcaccaccaggccggccgctgcctgctgtgggcgtgcaaggcctgcaagaggaagaccaccaacgctgaccgccgcaaagccgccaccatgagggaacggcggcggctcagcaaggtcaacgaggcctttgagaccctcaagcgctgcacttccaccaaccccaaccagcgcctgcccaaggtggagatcctgcgcaacgccatccgctacatcgagagcctgcaggccctgctgcgtgagcaggaggatgcatactacccagtgctggagcactacagcggggagtcagatgcctccagccctcgctccaactgctccgacggcatgatggagtacagcgggccgccctgtagctctcgcaggagaaacagctacgacagcagctactacacggaatcaccaaatgacccaaagcatgggaagagttctgttgtttccagcctcgactgcctctcaagcattgtggagaggatttccacagacaactccacatgtcccatactgcctccagctgaagctgtagctgaagggagtccctgttccccccaggaaggaggaaacctgagtgacagtggagcccagattccttcccccaccaactgcacccctcttccccaggaaagcagcagcagcagcagcagcaatccaatctaccaagtgctataaaggcaggtccagccggactgcaccgagaacaaattgctccgttcagccaagctccaagacctgccttctaaaagaggaaggacttcaagacttgttccagttttaaaatatcatgcaaaattccttctataacttttcaaacctgtattactacaaaatacacctagttatttattggttgctaaactaaagttatttaatatgtctagaaataaaagcgtatacggggaaatggccaatgtttaatttgggctttggagaatagggaacctggctcttgaatacggaggagaaaagaaatctacaacagcagtggtgtgacagatccttctccttattacccctgttctgccaaaataaggttgtggaccattttttataaaacttttgtataattgtaaataagagttgctttgcaaaaaagaaaaaaaaaaagaaaagaaaaaagaagaaaaaaaaaaaaaaaa

SEQ ID NO: 50 - protein, 298 aa, Gallus gallus
MDLLGPMEMTEGSLCSFTAADDFYDDPCFNTSDMHFFEDLDPRLVHVGGLLKAEEHPHTRAPPREPTEEEHVRAPSGHHQAGRCLLWACKACKRKTTNADRRKAATMRERRRLSKVNEAFETLKRCTSTNPNQRLPKVEILRNAIRYIESLQALLREQEDAYYPVLEHYSGESDASSPRSNCSDGMMEYSGPPCSSRRRNSYDSSYYTESPNDPKHGKSSVVSSLDCLSSIVERISTDNSTCPILPPAEAVAEGSPCSPQEGGNLSDSGAQIPSPTNCTPLPQESSSSSSSNPIYQVL

SEQ ID NO: 51 - DNA, 1074 bp, Gallus gallus
gagccgtgcacagtcctcccatggagctctttgagaccaacccttactttttcccggagcagaggttttacgatggggaaaacttcctgggctcccgcttgcagggctacgaggcggccgcgtttcctgagcgtcccgaggtgaccctgtgccctgaaagcagaggggctttggaggagaaggactcgacgctgcccgagcactgccccgggcaatgcttgccatgggcttgcaaaatctgcaagcgcaaaaccgtgtccatcgaccggcgtcgggcggccacgctgcgggagaagcggaggctgaagaaggtgaacgaagccttcgaggctctgaaacgcagcactctgctcaaccccaaccagcggctgcccaaggtggagatcctgcgcagcgccatccagtacatcgagcgcctgcagagcctgctcagcagcctcaaccagcaggagcgtgagcagagggagctgcgctaccgccccgctgcaccacaacctgctgcacccagcgagtgcggctctggcagctcatcctgcagccctgagtggagcacccagctggagtttggcaccaaccccgcagatcacctcctgagcgatgaccaggcagaggaccgcaacctccactcgctctcctccatcgtggagagcatcgccgtggaggacgtggccgtgacgttcccagaggagcgggtccaaaactgagctggcgcaaagccccgcggctctctgagctggaaacggggtgggatggtgatgctggaaggatccagcagctggaaatcataattacccctccgacactgtggactgcagcgctgaaaccgcccaaatcctttcccactcctctccaaacgttcccaccggtggaaaaaagaaaaggggagaagtgtgatggcggtggctggcggtggccctgggtcccccaggccgggggctttccttccttgggggccaacacgtgtgccacagccaatgtggctgtttccaagttgctcttcgaccaaggtctctctccggcgggctgccttcgccggctttcctactgaccgctgtgtgcagttgccatttgttt

SEQ ID NO: 52 - protein, 227 aa, Gallus gallus
MELFETNPYFFPEQRFYDGENFLGSRLQGYEAAAFPERPEVTLCPESRGALEEKDSTLPEHCPGQCLPWACKICKRKTVSIDRRRAATLREKRRLKKVNEAFEALKRSTLLNPNQRLPKVEILRSAIQYIERLQSLLSSLNQQEREQRELRYRPAAPQPAAPSECGSGSSSCSPEWSTQLEFGTNPADHLLSDDQAEDRNLHSLSSIVESIAVEDVAVTFPEERVQN

SEQ ID NO: 53 - DNA, 6067 bp, Gallus gallus
gtctccacggaaagcttcaagcattgaccagctgccaggctgcaagtcacccatctggtgtcacaagagagtcttctgctgaggtgtcttcagtgaagagcagccatggcttctccagatgctgaaatggccgcctttggggaggctgctccttatcttcgaaagtcagagaaggaaagaatcgaggcccagaacaggccttttaatgccaagtcatcagtctttgtggtgcatcccaaagaatcttttgtgaaagggactatccagagcaaagaaacagggaaggtcactgtcaagactgaaggtggagaaactctaactgtgaaggaagatcaaatcttctccatgaatcctcccaagtacgataaaattgaggacatggccatgatgacccaccttcacgaacccgctgtgctgtacaacctcaaagagcgttatgcagcctggatgatctacacctactcgggcctcttctgtgtcactgtcaacccctacaagtggctgccggtgtacaacccggaggtggtgttggcctaccgaggcaaaaagcgccaggaggcccctccacacattttctccatttctgacaacgcctatcagttcatgctgactgaccgtgagaatcagtcgatcctgatcaccggagaatctggtgcagggaagactgtgaacacgaaacgtgtcatccagtactttgcaacaattgcagctagcggggagaagaagaaggaggagcagcctggcaaaatgcagggaacgcttgaggatcaaatcatcagcgccaacccattgctagaggcctttggaaatgccaagactgtgaggaatgacaactcctcacgctttgggaaattcatcagaattcactttggtgccacaggaaaactagcttctgcagatattgaaacatatctattggagaagtctagagtcacttttcagctgaaggcagaaagaagctaccacatattttatcaaattatgtccaacaagaaaccagagctaattggcatgcttctaattaccaccaatccatacgactatcactttgtgagtcaaggtgagatcactgttcccagcattaatgatcaagaggagctgatggctacagatagtgccattgacatcctgggctttactgccgatgaaaaagtggctatttacaagctgacaggagctgtcatgcactatggcaacctgaagttcaagcagaaacaaagagaagagcaagcagagccagatggcacagaagttgcagacaaggctgcctacctgatgggtctgaactcagcagacctgctcaaggccctgtgctatcctcgtgtcaaggttgggaatgagtatgtgaccaagggtcaaactgtgcagcaggtgaacaactcagttggtgctctggcaaaggctgtctatgagaagatgttcttgtggatggttgttcgcatcaaccaacagttggataccaagcagcccaggcagtacttcattggtgtcctggacatcgctggatttgagatctttgatttcaacagcttggagcagctgtgcatcaacttcaccaatgagaaactgcaacagttcttcaaccaccacatgttcgtgctggaacaggaggagtacaagaaggaaggcattgaatgggagttcattgactttggaatggacttggctgcttgcattgagctcattgaaaagcccatgggcatcttctccatcctggaagaggagtgcatgttccccaaggcaactgacacctctttcaaaaacaagctctatgaccagcatctgggcaagtccagcaacttccagaagcccaagcctgccaaaggcaaggctgaggcccacttctctctggtgcactatgctggcacagtagactacaacatcactggctggctggagaagaacaaggaccccctgaacgaaactgtcattgggctgtaccagaaatcatctgtgaagacactggccttactgtttgccaactatggtggagcagaagcagaggctagtggtggtggtggtggtggcaagaaaggtggcaagaagaagggttcttctttccagactgtctcagcccttttccgggagaacctaaacaacttgatgaccaacctacggagtacacaccctcattttgtccgctgcatcatcccaaatgaaacaaaaacacctggtgccatggagcatgaactggtgctgcaccagctgcgctgtaacggcgtgctggaggggatcaggatttgcaggaaaggcttccccagcagagtcctctatgcagactttaaacagaggtacaaggtgcttaatgccagtgccatccctgagggacagttcattgatagcaagaaagcttctgagaagcttcttgggtcaattgatgtggaccacacccagtacaaatttggtcacaccaaggtgttcttcaaagctgggctgctgggactcctggaggagatgagagatgagaagctggcacagctcatcactcgcacacaggccaggtgcagaggcttcctgatgagagtggagtcccagagaatggtggagaggagggagtccatcttctgcattcagtcaatgttcggtgcattcatgaatgtcaaacactggccctggatgaagctgtccttcaagatcaagcccctgctgaagagcgcagagtctgagaaggagatggccaacatgaaggaagaatttgagaagaccaaggaagagcttgcaaagtctgaggcaaagaggaaggagctggaggagaaaatggtgaagctggtgcaggagaaaaatgatctgcagctccaagtgcaggctgaagctgatgctttggctgatgctgaggaaaggtgtgaccagctcatcaaaaccaaaatccagctggaagccaaaattaaggaggtgacagaacgggctgaggatgaggaggaaattaatgctgaactgacagccaagaagaggaaactggaggatgaatgctcagagctgaagaaagatatagatgacctggagttaacactggccaaagttgagaaggaaaagcatgccactgaaaacaaggtgaaaaacctcacagaagagatggcagccctggacgagacaattgccaagctgacaaaagagaagaaagccctccaagaggcccatcagcagacactggatgacctgcaggctgaagaggacaaagtcaatacgctgaccaaagctaaaaccaagctggaacagcaagtagacgatctggaagggtccctggagcaagagaagaagctgcgcatggacctcgagagagctaagaggaaacttgaaggagacctgaagatgtcccaggataccataatggacttggaaaatgataaacagcagctggatgagaaactgaagaagaaagactttgaaatcagccagatccagagcaaaattgaggatgagcaagccctgggcatgcagttacagaagaagatcaaggagctgcaggcccgtattgaggaactggaggaggaaatcgaggcagagcgaacctctcgggcaaaagcagagaagcaccgagctgacctctccagggagctggaggagatcagtgagcgcctggaggaagcaggaggggctacagcgactcagattgatatgaacaagaagcgtgaggcagaatttcagaagatgcgccgtgacctcgaagaggccacgctgcagcacgaagccacggctgctgccctgcggaagaagcatgcggacagcacagcggagcttggggagcagatcgacaacctgcaacgagtcaagcagaagctggagaaggagaagagtgagctgaagatggagattgatgacttggccagcaacatggagtctgtctccaaagctaaggcaaacctggaaaaaatgtgtcgtactttagaagaccaactgagtaagattaagtcaaaggaggaggagcatcaacgcatgatcaacgatctcagcactcaaagagctcgtctgcagacagaatcaggtgaatattcacgccaggtggaggagaaagatgccctgatatctcagctgtctcgaggcaagcaggcattcacccaacagattgaggaactcaagcggcacttagaggaagaaatcaaggccaagaacgccctggcccacgccttgcagtctgctcgtcatgactgtgatttgctccgggaacaatatgaagaggagcaggaagccaagggtgagctgcagcgtgccctgtccaaggccaacagtgaagtggcccagtggagaaccaaatatgagacagatgctattcagcgcacagaggaactggaggaggccaagaagaagctggcacagcgcctgcaggatgccgaggaacatgttgaagctgtgaatgccaaatgtgcttccctggaaaagacaaagcagaggctgcagaatgaggtggaggacctgatgattgacgtggagcgagcaaatgctgcctgcgcacgtctggacaagaagcagaagaactttgacaagatcctggcagagtggaagcagaagtacgaggaaacgcaggctgagctggaggcctcccagaaggagtctcgctccctcagcacagagctgtttaagatgaagaatgcctatgaggaatccctggaccacctggaaacgctgaagcgtgagaacaagaacttgcagcaggagatttctgacctcacagagcagattgcagagggaggaaaggcgattcatgagttggagaaagtcaagaagcagattgagcaggagaagtctgaaatccaggcagcactggaggaagctgaggcctccctggagcatgaagaggggaagatcctgcgcctccaactggagctcaaccaggtcaagtctgagattgacaggaagatagcagagaaagatgaggaaattgaccagctgaagagaaatcacctcagaattgtggagtccatgcagagcactctggatgctgagatcaggagcaggaatgaggccctgcggctgaagaagaagatggagggagacctgaatgaaatggagatccagctcaaccatgccaaccgcgtggctgcagaggcacaaaagaaccttagaaacacacaaggagtgctcaaagatacccagatacacttggatgatgctctcaggacacaggaggacctgaaggagcaggtggccatggtggagcgcagagcaaacctgttgcaggctgagattgaggagctacgggcagccctggaacagacagagcgctcgagaaaagtggctgagcaggaattgatggatgcaagtgagagagttcagctcctccacactcagaacaccagcttgatcaacaccaagaagaaactggaaacagacattgcccaaattcagagtgaaatggaggataccatccaggaagcccgcaatgctgaagagaaggccaagaaggccatcaccgatgcagccatgatggcagaagagctgaagaaggagcaggacaccagtgcccacctggagaggatgaagaagaacctggaccagacggtgaaggatctgcagctccgtctggatgaggcagagcagctggccctgaagggaggcaagaagcaaattcagaagctggaggccagggtgcgggagctggaaggggaggttgatgctgagcagaagcgcagcgctgaagccgtgaagggtgtgcgcaagtacgagaggagggtgaaggagctgacataccagtctgaggaagatcgcaagaacattcttagactccaggatctggtggacaaactgcagatgaaggtgaaatcctacaagagacaagctgaggaggctgaggagctgtccaatgtcaacctcaccaagttccgcaagatccagcatgagctggaggaagccgaggagcgggctgacattgcagagtcacaggtcaacaagctccgagcaaagagccgggagtttcatggcaagaagatagaagaggaagagtgaagatatcatctgacagcaaagtggcctgaggagtgcacaaaatgtgaaccctctgttgctatcacttataatttatctttataaccacctcaatgtctagagaataaagaccatagattcctctgca

SEQ ID NO: 54 - protein, 1944 aa, Gallus gallus
MASPDAEMAAFGEAAPYLRKSEKERIEAQNRPFNAKSSVFVVHPKESFVKGTIQSKETGKVTVKTEGGETLTVKEDQIFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGEKKKEEQPGKMQGTLEDQIISANPLLEAFGNAKTVRNDNSSRFGKFIRIHFGATGKLASADIETYLLEKSRVTFQLKAERSYHIFYQIMSNKKPELIGMLLITTNPYDYHFVSQGEITVPSINDQEELMATDSAIDILGFTADEKVAIYKLTGAVMHYGNLKFKQKQREEQAEPDGTEVADKAAYLMGLNSADLLKALCYPRVKVGNEYVTKGQTVQQVNNSVGALAKAVYEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSSNFQKPKPAKGKAEAHFSLVHYAGTVDYNITGWLEKNKDPLNETVIGLYQKSSVKTLALLFANYGGAEAEASGGGGGGKKGGKKKGSSFQTVSALFRENLNNLMTNLRSTHPHFVRCIIPNETKTPGAMEHELVLHQLRCNGVLEGIRICRKGFPSRVLYADFKQRYKVLNASAIPEGQFIDSKKASEKLLGSIDVDHTQYKFGHTKVFFKAGLLGLLEEMRDEKLAQLITRTQARCRGFLMRVESQRMVERRESIFCIQSMFGAFMNVKHWPWMKLSFKIKPLLKSAESEKEMANMKEEFEKTKEELAKSEAKRKELEEKMVKLVQEKNDLQLQVQAEADALADAEERCDQLIKTKIQLEAKIKEVTERAEDEEEINAELTAKKRKLEDECSELKKDIDDLELTLAKVEKEKHATENKVKNLTEEMAALDETIAKLTKEKKALQEAHQQTLDDLQAEEDKVNTLTKAKTKLEQQVDDLEGSLEQEKKLRMDLERAKRKLEGDLKMSQDTIMDLENDKQQLDEKLKKKDFEISQIQSKIEDEQALGMQLQKKIKELQARIEELEEEIEAERTSRAKAEKHRADLSRELEEISERLEEAGGATATQIDMNKKREAEFQKMRRDLEEATLQHEATAAALRKKHADSTAELGEQIDNLQRVKQKLEKEKSELKMEIDDLASNMESVSKAKANLEKMCRTLEDQLSKIKSKEEEHQRMINDLSTQRARLQTESGEYSRQVEEKDALISQLSRGKQAFTQQIEELKRHLEEEIKAKNALAHALQSARHDCDLLREQYEEEQEAKGELQRALSKANSEVAQWRTKYETDAIQRTEELEEAKKKLAQRLQDAEEHVEAVNAKCASLEKTKQRLQNEVEDLMIDVERANAACARLDKKQKNFDKILAEWKQKYEETQAELEASQKESRSLSTELFKMKNAYEESLDHLETLKRENKNLQQEISDLTEQIAEGGKAIHELEKVKKQIEQEKSEIQAALEEAEASLEHEEGKILRLQLELNQVKSEIDRKIAEKDEEIDQLKRNHLRIVESMQSTLDAEIRSRNEALRLKKKMEGDLNEMEIQLNHANRVAAEAQKNLRNTQGVLKDTQIHLDDALRTQEDLKEQVAMVERRANLLQAEIEELRAALEQTERSRKVAEQELMDASERVQLLHTQNTSLINTKKKLETDIAQIQSEMEDTIQEARNAEEKAKKAITDAAMMAEELKKEQDTSAHLERMKKNLDQTVKDLQLRLDEAEQLALKGGKKQIQKLEARVRELEGEVDAEQKRSAEAVKGVRKYERRVKELTYQSEEDRKNILRLQDLVDKLQMKVKSYKRQAEEAEELSNVNLTKFRKIQHELEEAEERADIAESQVNKLRAKSREFHGKKIEEEE

SEQ ID NO: 55 - DNA, 1849 bp, Mus musculus
acctggttgatcctgccagtagcatatgcttgtctcaaagattaagccatgcatgtctaagtacgcacggccggtacagtgaaactgcgaatggctcattaaatcagttatggttcctttggtcgctcgctcctctcctacttggataactgtggtaattctagagctaatacatgcccacgggcgctgaccccccttcccggggggggggggggatgcgtgcatttatcagatcaaaaccaacccggtgagctccctcccggctccggccgggggtcgggcgccggcggctttggtgactctagataacctcgggccgatcgcacgcccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagtcaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggagtttcgaagacgatcagataccgttgtagttccaaccataaacgatgccgactggcaatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggccataagcttgcgttgattaagtccctgccctttgtacacaccgcccgtcgctactaccgattggatggtttagtgaggcccacggccctggtggagcgctgagaagacggtcgaacttgactatctagaggaagtaaaagtcgtaacaaggtttccgtaggtgaacctgcggaaggatcattaa

SEQ ID NO: 56 - protein, 9 aa, Artificial Sequence
  The sequence has been designed and synthesized.
YPYDVPDYA

SEQ ID NO: 57 - DNA, 4944 bp, Mus musculus
ggcggcccgcggtttgggccggtttgccagcctttggagcgaccgggagcatataactggagcctctgccgggggaagacgcagagcgccgctgggctgccgggtctcctgcctcctcctgctcctagggcctcctgcatgagggagcggtagagacccggacccgctccgtgctctgccgcctcgctgcgcttcgcccgggccaggctctgccaggcctcgcggtgagccatgatccgcctcggggctccccagtcgctggtgctgctgacgctgctcatcgccgcggtcctacggtgtcagggccaggatgcccgaaaattagggccaaaggggcagaaaggagaacctggagatatcagagatatcataggacccagaggacctcctggccctcagggacctgcaggtgaacaaggacccagaggtgatcgtggtgacaagggagaaaagggtgcgcctggaccccgtggcagagatggagaacctggtacccctggaaatcctggccccgctggccctccaggtccccctggtccccctggccttagtgcaggaaacttcgcggctcagatggctggagggtatgacgagaaggctggtggtgcccagatgggagtcatgcaagggcccatgggccccatgggaccccgtggacccccaggccctgccggtgcccccggccctcaaggatttcaaggcaatcctggtgaacctggcgagcctggtgtctctggtcccatgggtccccgaggtcctcctggccctgctggaaaacctggtgacgacggtgaagctgggaagcccggaaagtctggggaaagaggcctccctggccctcagggtgctcgtggattcccaggaaccccgggtctccccggtgtcaagggtcacagaggttacccaggcctcgacggtgctaagggggaagctggtgctccgggtgtgaagggtgagagtggttcccctggtgagaacggatccccgggcccaatgggtccccgtggcctgcctggtgagagaggacggactggccctgctggtgctgctggtgctcggggtaacgatggccagccaggccccgctggacctccgggtcctgtgggtcccgcaggtggtcctggcttccctggtgctcctggtgccaagggcgaagctggtcccactggtgctcgcggtcctgaaggtgctcaaggttctcgtggcgagcctggcaatcctgggtcccctgggcctgcaggtgcttctggtaacccagggactgatggtattcctggagccaaaggatccgctggtgctcctggaattgctggtgcccctggcttccctgggccccgtggccctcccggtcctcaaggtgcaactggtccccttggccccaaaggtcaggcgggtgaacctggcattgctggctttaaaggtgatcaaggccccaagggagagactggacctgctgggccccaaggagcccctggccccgctggtgaagaaggcaaacgaggtgctcgaggagagccgggtggtgctggaccaatcggaccccctggagagagaggtgctcctggcaaccgtggattcccaggtcaagatggtctggcaggtcccaagggtgcccctggagagcgagggcccagtggcttgactggtcccaagggagccaacggtgacccgggtcgtcctggagaacctggtcttcctggagccaggggtcttaccggtcgccctggtgacgctggtcctcaaggcaaagttggtccttctggagcccctggtgaagacggtcgccctggacctcctggtcctcagggagctcgtgggcagcctggcgtcatgggtttccctggccccaaaggtgccaacggcgagcctggcaaagctggtgagaagggtctggctggcgctcctggtctgagaggtcttcctggaaaagacggtgagacgggagccgcaggaccccccggccccagtggacctgctggtgaacgaggcgagcagggcgctcctggaccatcagggttccagggacttcctggccctcccggtcccccaggtgaaggtggaaagcaaggtgaccagggtattcctggtgaagctggagctcctggccttgtgggtcctcggggcgagcgaggtttcccaggtgaacgtggctctcctggtgctcagggccttcagggtccccgaggcctccctggcactcctggtactgatggtcccaaaggtgcagctggcccagatggcccccctggggctcaggggcctccaggtctacagggaatgcctggtgagagaggagccgctggcattgctgggcccaagggagacagaggcgatgttggcgagaaaggcccagagggagctcctgggaaggatggcggccgaggtctgactgggcccatcggacccccaggcccagcaggggccaacggcgagaagggagaagtcggacctcctggcccgtcaggaagtaccggagctcgaggtgccccgggtgaacgcggagagaccgggccacctggacctgctggattcgctggccctcctggtgctgatggccagcctggtgccaagggtgatcaaggagaagccggacagaaaggagatgctggtgcccccggcccacaaggcccctcgggagcccctgggccacagggtcctactggagtgactggtcctaagggagcccgaggtgcccaaggtcccccgggagccaccggattccctggagctgctggccgagttggacccccaggtgctaatggcaatcctggacccgccggtccccctggtcctgctggaaaagatggtcccaaaggtgttcgaggagacagtggcccccctggcagagctggtgaccccggtcttcaaggtcctgcaggagctcctggcgagaaaggagaacctggagatgatggtccctctggtcttgatggtcctccaggtccccaggggctggctggtcaaaggggcattgttggtctgcctggtcagcgtggtgagagaggattccccggccttcccggcccatcgggtgagcccggcaagcagggtgcacctggcgcgtctggagacagaggtcctcctggtcctgtggggcctcctggcctgacagggcctgcaggtgaacctggacgagagggcagccctggtgctgatggaccccctggaagagatggtgcagctggagtcaagggagatcgtggtgagactggagcactgggtgcccctggagctcctgggcccccaggctctcctggtcctgctggcccaactggcaaacaaggagacagaggagaggctggtgcacaaggtcctatgggtccctcaggacctgctggagcccgtgggattgcaggccctcaaggcccccgaggtgacaaaggagaatctggagagcagggcgagaggggactgaagggacaccgaggtttcactggactgcagggtctgcctggccctccgggtccttctggagatcagggtgcttctggccctgctggtccttctggccctagaggtccacctggccctgttggtccctctggcaaagatggctctaatggaatccctggccccatcgggcctccaggtccccgtggacgctcaggagaaacaggccctgttggtccccctggaagtcccggtcctcctggccctccaggtcctcctggtcctggcatcgacatgtcagcctttgctggcttagggcagagagagaagggccccgaccccatgcagtacatgcgggccgacgaggcagacagtaccttgagacagcacgacgtggaggtggacgctacactcaagtcactgaacaaccagattgagagcatccgcagccccgacggctcccgcaagaaccctgctcgcacttgccaagacctgaaactctgccaccccgagtggaagagcggagactactggattgatcccaaccagggctgcaccttggacgccatgaaagttttctgcaacatggagaccggcgagacttgcgtctaccccaacccagcgactgtccctcggaaaaactggtggagcagcaagagcaaggaaaagaaacacatctggtttggagagaccatgaacggtggcttccacttcagctatggcgatggcaacctggctcccaacaccgctaacgtccagatgactttcctccgtctactgtccactgagggctcccagaacatcacctaccactgtaagaacagcatcgcctacctggacgaagcggctggcaacctcaagaaggccttgctcatccagggctccaatgatgtagagatgagggccgagggcaacagcaggttcacatacactgccctgaaggatggctgcacgaaacacactggtaagtggggcaagaccgtcatcgagtaccgatcacagaagacctcccgccttcccattattgacatcgcacccatggacattggaggggctgaacaggaatttggtgtggacatagggcctgtctgcttcttgtaaaacccccgaaccctgaaacaacacaatccattgcgaacccaaaggacccaaacactttccaaccgcagtcactccaggatctgcactgaatggctgacctgacctgatgatacccaaccgtcctcccctcacagcccggactgtgctcccctttctaagagacctgaactgggcagactgcaaaataaaatctcggtgttctatttatttattgtcttcctgtaagacctctgggtccaggcggagacaggaactatctggtgtgagtcagacgccccccgagtgactgttcccagcccagccagaagacccctacagatgctgggcgcagggactgcgtgtcctacacaatggtgctattctgtgtcaaacacctctgtattttttaaaacatcaattgatattaaaaaccaaaaaaaaaaaaatcattggaaaggaaaaaaaaaaaaaaaa

SEQ ID NO: 58 - protein, 1419 aa, Mus musculus
MIRLGAPQSLVLLTLLIAAVLRCQGQDARKLGPKGQKGEPGDIRDIIGPRGPPGPQGPAGEQGPRGDRGDKGEKGAPGPRGRDGEPGTPGNPGPAGPPGPPGPPGLSAGNFAAQMAGGYDEKAGGAQMGVMQGPMGPMGPRGPPGPAGAPGPQGFQGNPGEPGEPGVSGPMGPRGPPGPAGKPGDDGEAGKPGKSGERGLPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGSRGEPGNPGSPGPAGASGNPGTDGIPGAKGSAGAPGIAGAPGFPGPRGPPGPQGATGPLGPKGQAGEPGIAGFKGDQGPKGETGPAGPQGAPGPAGEEGKRGARGEPGGAGPIGPPGERGAPGNRGFPGQDGLAGPKGAPGERGPSGLTGPKGANGDPGRPGEPGLPGARGLTGRPGDAGPQGKVGPSGAPGEDGRPGPPGPQGARGQPGVMGFPGPKGANGEPGKAGEKGLAGAPGLRGLPGKDGETGAAGPPGPSGPAGERGEQGAPGPSGFQGLPGPPGPPGEGGKQGDQGIPGEAGAPGLVGPRGERGFPGERGSPGAQGLQGPRGLPGTPGTDGPKGAAGPDGPPGAQGPPGLQGMPGERGAAGIAGPKGDRGDVGEKGPEGAPGKDGGRGLTGPIGPPGPAGANGEKGEVGPPGPSGSTGARGAPGERGETGPPGPAGFAGPPGADGQPGAKGDQGEAGQKGDAGAPGPQGPSGAPGPQGPTGVTGPKGARGAQGPPGATGFPGAAGRVGPPGANGNPGPAGPPGPAGKDGPKGVRGDSGPPGRAGDPGLQGPAGAPGEKGEPGDDGPSGLDGPPGPQGLAGQRGIVGLPGQRGERGFPGLPGPSGEPGKQGAPGASGDRGPPGPVGPPGLTGPAGEPGREGSPGADGPPGRDGAAGVKGDRGETGALGAPGAPGPPGSPGPAGPTGKQGDRGEAGAQGPMGPSGPAGARGIAGPQGPRGDKGESGEQGERGLKGHRGFTGLQGLPGPPGPSGDQGASGPAGPSGPRGPPGPVGPSGKDGSNGIPGPIGPPGPRGRSGETGPVGPPGSPGPPGPPGPPGPGIDMSAFAGLGQREKGPDPMQYMRADEADSTLRQHDVEVDATLKSLNNQIESIRSPDGSRKNPARTCQDLKLCHPEWKSGDYWIDPNQGCTLDAMKVFCNMETGETCVYPNPATVPRKNWWSSKSKEKKHIWFGETMNGGFHFSYGDGNLAPNTANVQMTFLRLLSTEGSQNITYHCKNSIAYLDEAAGNLKKALLIQGSNDVEMRAEGNSRFTYTALKDGCTKHTGKWGKTVIEYRSQKTSRLPIIDIAPMDIGGAEQEFGVDIGPVCFL

SEQ ID NO: 59 - DNA, 1437 bp, Mus musculus
gaagagataatcggccgggctgtaggagaagagcatctggcgctggattccaggggagggggtcctggcgaggtgggcctgtgaggtggggtgagggtcggcgcggatcctggaactgaggggggcgggggctgctgtgctgcgcaggagtgggttgcgggggcctggcgcgcccctctcactcgcgctgaggtcgcccagagctccgtgtcaggctcgaagctgtccccgcgccccagttgccctcctggctgccggctccgatctgcggcgcagatggctgtgcgcggcagcggcaccttgacgcccttctccatccaggcgatcctcaacaagaaagaggagcgcggcgggttggctacgccggaggggcgcccggcaccagggggcacagaggtggcggtgaccgcggcgcccgccgtctgctgctggcggatcttcggggagaccgaggccggtgcgctggggggcgccgaggactctcttctggcatctcccgcccgaaccagaacagccgtggggcagagcgcagagagtccgggaggttgggactcagactcagccctgagcgaagagaacgagggcaggaggcgctgcgccgatgtcccgggagccagtggaaccggccgcgccagggtgaccctgggcctggaccagccaggctgcgagctgcacgccgccaaggacctggaggaggaagccccggtccgcagcgacagcgagatgtcagccagcgtttcaggcgaccacagcccgagaggcgaggatgacagcgttagccctggaggtgcacgcgtgcctgggttgcgcggcgccgcgggcagcggggctagcggcgggcaggcgggcggcgtggaggaggaggaagagcccgcagcccctaaaccgcgaaagaagcgctcccgggccgccttctcgcacgcccaggtcttcgagctggagcgccgctttaaccatcagcgctacctgtccgggccggagcgcgcagacctggcagcttcgctgaagctcacagagacgcaagtgaagatctggtttcagaaccgtcgctacaagaccaaacgccggcagatggccgccgacctgctcgcctctgcacccgccgccaagaaagtggccgtaaaggtgctggtgcgtgacgaccaaagacagtatttgcccggagaggtgctgaggccaccttcccttctgccactgcagccctcctactactacccttactactgtctcccgggctgggcgctgtccacgtgcgcggccgctgctggtacccagtgaagcccttgggacgaggcaaagaaggattcctgcgtcccccgtatgcaccgccacggacaggtgtactctgtgcaggcgagttctgctcaactggggatgaggcaggtggggaatgaaatcctgctgggacttgacacacctatccagggtgacagggcc

SEQ ID NO: 60 - protein, 333 aa, Mus musculus
MAVRGSGTLTPFSIQAILNKKEERGGLATPEGRPAPGGTEVAVTAAPAVCCWRIFGETEAGALGGAEDSLLASPARTRTAVGQSAESPGGWDSDSALSEENEGRRRCADVPGASGTGRARVTLGLDQPGCELHAAKDLEEEAPVRSDSEMSASVSGDHSPRGEDDSVSPGGARVPGLRGAAGSGASGGQAGGVEEEEEPAAPKPRKKRSRAAFSHAQVFELERRFNHQRYLSGPERADLAASLKLTETQVKIWFQNRRYKTKRRQMAADLLASAPAAKKVAVKVLVRDDQRQYLPGEVLRPPSLLPLQPSYYYPYYCLPGWALSTCAAAAGTQ

SEQ ID NO: 61 - DNA, 4146 bp, Mus musculus
tgagctggaagtcggagagccgagagcgctgctcggaactgcctggaaacttctgtgggagcgacaactttaccagtttcagtccaggaacttttctttgcaagagagacgaggtgcaagtggccccggtttcgttctctgttttccctccctcctcctccgctccgactcgccttccccgggtttagagccggcagctgagacccgccacccagcgcctctgctaagtgcccgccgccgcagcccggtgacgcgccaacctccccgggagccgttcgctcggcgtccgcgtccgggcagctgagggaagaggagccccagccgccgcggcttctcgcctttcccggccacccgccccctgccccgggctcgcgtatgaatctcctggaccccttcatgaagatgaccgacgagcaggagaagggcctgtctggcgcccccagccccaccatgtcggaggactcggctggttcgccctgtccctcgggctccggctcggacacggagaacacccggccccaggagaacaccttccccaagggcgagccggatctgaagaaggagagcgaggaagataagttccccgtgtgcatccgcgaggcggtcagccaggtgctgaagggctacgactggacgctggtgcccatgcccgtgcgcgtcaacggctccagcaagaacaagccacacgtcaagcgacccatgaacgccttcatggtgtgggcgcaggctgcgcgcaggaagctggcagaccagtacccgcatctgcacaacgcggagctcagcaagactctgggcaagctctggaggctgctgaacgagagcgagaagagacccttcgtggaggaggcggagcggctgcgcgtgcagcacaagaaagaccaccccgattacaagtaccagccccggcggaggaagtcggtgaagaacggacaagcggaggccgaagaggccacggaacagactcacatctctcctaatgctatcttcaaggcgctgcaagccgactccccacattcctcctccggcatgagtgaggtgcactccccgggcgagcactctgggcaatctcagggtccgccgaccccacccaccactcccaaaaccgacgtgcaagctggcaaagttgatctgaagcgagaggggcgccctctggcagaggggggcagacagccccccatcgacttccgcgacgtggacatcggtgaactgagcagcgacgtcatctccaacattgagaccttcgacgtcaatgagtttgaccaatacttgccacccaacggccacccaggggttccggccacccacggccaggtcacctacactggcagttacggcatcagcagcaccgcacccacccctgcgaccgcgggccacgtgtggatgtcgaagcagcaggcgccgccccctcctccgcagcagcctccgcaggccccgcaagccccacaggcgcctccgcagcagcaagcacccccgcagcagccgcaggcaccccagcagcagcaggcacacacgctcaccacgctgagcagcgagccaggccagtcccagcgaacgcacatcaagacggagcagctgagccccagccactacagcgagcagcagcagcactccccgcaacagatctcctacagccccttcaaccttcctcactacagcccctcctacccgcccatcacccgctcgcaatacgactacgctgaccatcagaactccggctcctactacagtcacgcagccggccagggctcagggctctactccaccttcacttacatgaaccccgcgcagcgccccatgtacacccccatcgctgacacctccggggtcccttccatcccgcagacccacagcccgcagcactgggaacaaccagtctacacacagctcaccagaccctgagaagagaaaagctatggtgacagagctgatcttttttttttttttttttaaagaagaaaagaaagaaacgaaaagaaaaagctgaagaaatcaagaaccaattgaaattcctttggacacttttttttttttgtctgtcgttaatttttaaagacatgtaaaggaaggtaacgattgctgggattccaggagagagactttaagactttgtctgagctcatgacaacatattgcaaatggccgggccactcgtggccagacggacagcactcctggccagatggacccaccagtatcagcgaggaggggcttgtctccttcagagttaacatggaggacgattggagaatctccctgcctgtttggactttgtaattattttttagccgtaattaaagaaaaaaaaagtcctctgtgaggaatattctctattttaaatacttttagtatgtactgtgtatgactcattaccattttgaggggatttatacatattttagataaaaaaagttaaatgctcttatttttccaacagcttaactactcctagttcaagagtgtcccttagccttccttgcaaccagagtatttttgtacagatttgctttctcctaaacaaacaaacaaaaacaaaaacaaaaaaaacaaaaaaaaatgaaaaaaaaaaagaaaaatgaaaaaagtgtgttgttatagtaacataaataatacacagaattttgttacactatcagtttggggggtgagctttgattaattccccaggctcttggatttcaagagtagctgccttaaaagaaagaaagaaaaaagaaagaaaggaaggaaggaaggaaggaagggagagagagagagaaagccttcttttgcaataaggggaataaacagataacatagagcattggtgagctttatcatatatatatatatatttttaaaaaacgagaagaaaacaccttgagccttaaatcccggctgctgggaaagtatatgctgttcctagaacattcactgtgcctttgcctgcttgctggctgcagcctcagaaggaaagccaggggcaagcaaaggagaccaaaatttcttggggaagatttttcacgcagccctaagtgcccaagcacattttccctggttgcctcctgggccccatgtggccagcagatgctggagtctgcctgctcagactatcacctgtacctccctgaataccagcgtttaaccttcaagacatcccatgtgctaaaattatttattttgtaaggagaagttttcatttttttttttttaaaaaaaatctccttcctttttttctttcttgtttttctttttcttcttcttcttcttctttttttttttaatttactgtctttaaggtagattgttggcgccttcctcaaagggtatggtcatctgttgttaaattatgttcttaactgtaaccttttgttgttgttgtttttaatttatctctttaatcttttttttattagtaaaagcaagtctctttgaattcttcattctagatttgtacaaatgctttttttgtccgtcctcgtggggttttttttctcccccttttctttgttgttttcgttgttgttgataacaaactggaaacctgtctctctctgtttgtggacaaatgagaggtttcagatgcagtgaggagcactgagtcctttgcaatgttttcagccatagacctttgggtctgcctggactgtatgtggatgtgtgcgtgtgttgtgacacgggacaacacatgcctctgcaagtgtgtgtgccgtggatagccccttggctgctctcctgcagagagacatcggacagaccttaattcttactcactgctgtggctggagagtataaggaatgctttttctttttttctttctttctttcttttttttttttaagacagcagtctttttttttaatttaaaaaaaaaaagatatattaacagttttagaagtcagtagaataaaaccttaaagcgttcttataatatggcatctttcgatttctgtataaaaacagaccttttaaaaaatatttctgtaacttaagaaacctgacatttatgtcatattttctctttaggtaagatttggtttgtttgtgtcttgtttgtttccctctccaaattcttccttctttgtgcaccctgcctttcttcccttccatcctttcttttttgtatattattgtttacaataaatatacattgcattaaagtgaaaaca

SEQ ID NO: 62 - protein, 507 aa, Mus musculus
MNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQAGKVDLKREGRPLAEGGRQPPIDFRDVDIGELSSDVISNIETFDVNEFDQYLPPNGHPGVPATHGQVTYTGSYGISSTAPTPATAGHVWMSKQQAPPPPPQQPPQAPQAPQAPPQQQAPPQQPQAPQQQQAHTLTTLSSEPGQSQRTHIKTEQLSPSHYSEQQQHSPQQISYSPFNLPHYSPSYPPITRSQYDYADHQNSGSYYSHAAGQGSGLYSTFTYMNPAQRPMYTPIADTSGVPSIPQTHSPQHWEQPVYTQLTRP

SEQ ID NO: 63 - DNA, 3868 bp, Mus musculus
gctccctagtccggatccctgcactcggtgtcacgacgggaggagacttgggacgtgttcctgcctcgtcaccaccctccctcacccgatcttcgcggtgcagtgagcacctttgccagtagccccagctgaggggccgatctgctagactcgcaccaaactcgtccgccctgggcttgggatcctgcactcaagggactcctctggagcctgtggacttggatctattagcgcccccctccccgcgccccgcctccctctctggccttttttgggggaggagctctccgagatccggagagttcccgagggtcacccgccgcactgtgctcgctttttcgtctcgccttcacctggatataatttgcgagcgaagctgcccccaggatgaccacgctggccggcgctgtgcccaggatgatgcggcccggcccggggcagaattacccacgcagcggcttcccgctggaagtgtccacccctcttggccagggccgagtcaaccagctcggaggagtatttatcaacggcaggcctctgcccaaccatatccgccacaagatagtggagatggcccaccatggcattcggccttgcgtcatttctcgccagcttcgcgtgtcccatggttgcgtctctaagatcctgtgcaggtaccaggagacaggctccatccgacctggtgccatcggcggcagcaaacccaagcaggtgacaacgcctgacgtggagaagaaaattgaggaatacaaaagagagaacccgggcatgtttagctgggaaatcagagacaaattgctcaaggacgctgtctgtgatcggaacactgtgccctcagtgagttctatcagccgcatcctgaggagtaaatttggaaaaggagaagaggaggaggcggatctagaaaggaaggaagcagaagaaagcgagaaaaaggctaaacacagcatcgatggcatcctgagtgagcgagcctctgcacctcagtcagatgaaggctccgatattgactctgaacctgatttaccgctgaagaggaagcagcgcaggagcagaaccaccttcacggcagagcagctggaggaactggagcgggctttcgagagaacccactacccagacatttacaccagggaggagctggcccagagggcgaagcttaccgaggcccgagtgcaggtctggtttagcaaccgccgtgcaagatggaggaaacaagctggagccaatcaactgatggctttcaaccatctcattccggggggattccctcccaccgccatgccgaccctgccaacataccagctgtcggagacctcttaccagcccacgtctattccacaagccgtgtcagatcccagtagcaccgtccacagacctcagccgcttcctccgagcactgtacaccaaagcactattccttcgaacgcagacagcagctctgcctactgcctccccagcaccaggcatggattttcaagctatacagacagctttgtgcctccatcggggccctccaaccccatgaaccccaccatcggcaatggcctttcacctcaggtaatgggacttctgaccaaccacggtggggtaccgcaccagcctcagaccgactatgctctctcccctctcactgggggcctggaacccacgaccacggtgtcagccagctgcagtcagagactggaacatatgaagaatgtggacagtctgcccacatctcagccctattgtccccccacctatagcaccgcaggctacagtatggaccctgtcacaggctaccagtatgggcagtatggacaaagtaagccttggacgttctagggggtagttcctcctggaagggagagagatcacctcttgcttgagaacggggaactggaggcatgtttaagcctttcatcccagtatcatttttttgtggcaaagacagctgattgctacagcacaggctcccttgtgtaattatttgcttaactgatgtcaataacatcttgcagttattaattgctgggacggaaaaccggattgtcagtaggtaaaacacagaggttggccaaaatgaaataacctctagcattagaaacacatgttcttaatgaggtcagcgccaggatcatatggggataagcccaggacacagagttgtgtcaaacttgtctcaggaataaaaatattagtctcgatcctttgataccgtggtattaaatatgacattgtcagcctgtagctgatcttgcccctaactgtgaattgtcccagcatgacctaaaaagctgcgtgtgtttccttacaggtgcctttcattatctcaagccagatattgcataagtgaactgtccacttggagccctgtttccggcctccacagactagacatgaagaatcttctcagaaacaaaacaataaaaaaaatttaaaaacaaacaaaaaataaatgaaaaaaaattcagcttttgggggaggtggagacaaaagagattgattttctttctccagtagttgctttcagattctttgaacatattggacaaaaggcaggggagacaaggaagacccagagctctaaaatgcaccatttttaagacagtgatgtaactggctacatatcaaaatggccccaaatctttgttgctgatcaaagaagctaaaacattccgtgtgtgtgtgtgtgtgtgtgtgtgtgtgagagagagagagagagagagagagagagagagagattgattttcacggggtggtttgagtaagcagaatgttcccatatattgtagttgaattcctgaaagttaacaggtttgatgtctaaggctgaccaagtttatgacattcacactgcttatgtagtgaattgggaatggagaaatccttgattttgacacactggaaaccgatatgaaaaccaacaccaccattgtatagggcactagactagatgtgaaggatctccccagtaaattattaccataccttcatttgtaacctagttatcaaccgatgtccctgacatgtcttgtaaaaagatgtgggataatgagaaaaaagtgtgttatctgctgtggggatagatggatgtcatttacttcccaagtcacattgatttggaggccagttggtgggggggggggaagggtatgatgtagaggaagtgacacccatcacatatctgcttctcagcgtgcaatactgtgtacctcacagatgctttggcaatggccaaggtgatagaaatgaaatcagaactatatttttgcaggtgtttttatttttgcagcccataactcatcgtagtagagtgcagtgatgtttgctcgttgccaaacaactcacatcctgcttgttccttttgttcagagaccatgtctgagaatcaagctctctacagagatggtgtttacattcgtctcttttctacaaaaggcagggctagaacagaaatgtccacgtggctctcatgtacttttaaattatacaacgatacccaatattaaaggatatagggaacaggtttacatacattcatgtggtgacatttaggatgtcaataaatctgtgttttgaaatgttaagagaaagatgaaggtgggcggtgctgttatttatttctttctattcttggaaccatgaatgaggtaggcacaaatacattccctttagatttaagaacaatgaggtagtgtgttagcaggactagacatagaactgagttattttatacgttgggcagggtgatggaacaggagacatagtatctttgtgttgcagcctcaaccaacaatgatgtgttgtaaaaatgtctttcatgtaaaaagtgcagtaaatatttactatttataatgaataaacagttaatgaaaatgtgccc

SEQ ID NO: 64 - protein, 479 aa, Mus musculus
MTTLAGAVPRMMRPGPGQNYPRSGFPLEVSTPLGQGRVNQLGGVFINGRPLPNHIRHKIVEMAHHGIRPCVISRQLRVSHGCVSKILCRYQETGSIRPGAIGGSKPKQVTTPDVEKKIEEYKRENPGMFSWEIRDKLLKDAVCDRNTVPSVSSISRILRSKFGKGEEEEADLERKEAEESEKKAKHSIDGILSERASAPQSDEGSDIDSEPDLPLKRKQRRSRTTFTAEQLEELERAFERTHYPDIYTREELAQRAKLTEARVQVWFSNRRARWRKQAGANQLMAFNHLIPGGFPPTAMPTLPTYQLSETSYQPTSIPQAVSDPSSTVHRPQPLPPSTVHQSTIPSNADSSSAYCLPSTRHGFSSYTDSFVPPSGPSNPMNPTIGNGLSPQVMGLLTNHGGVPHQPQTDYALSPLTGGLEPTTTVSASCSQRLEHMKNVDSLPTSQPYCPPTYSTAGYSMDPVTGYQYGQYGQSKPWTF

SEQ ID NO: 65 - DNA, 4275 bp, Mus musculus
agagacgccaagaggtttatccagccgactctggattcgtctccagcgtgcgcaggaatggcggcgctgcccggcgcggtccccaggatgatgagacccggcccggggcagaactacccgcgcaccggcttccccctggaagtgtccacccctcttggccaaggccgggtcaatcagcttggtggggtcttcatcaacggtcgacccctgccgaaccacatccgtcacaagatagtggaaatggcccaccatggcatccggccctgcgtcatctcccgtcagctccgtgtttcccatggttgtgtctccaagattctgtgccgatatcaggagactgggtccatccggcccggggctatcggaggcagcaagcccagacaggtggcgactccggatgtggagaaaaagattgaggagtataagagagagaaccccgggatgttcagctgggaaatccgggaccggctgctgaaggacggtcactgcgaccgaagcacggtgccctcagtgagttcgattagccgagtgctcagaatcaagttcgggaagaaagaggacgacgaggaaggagacaagaaagaagaagatggcgagaagaaagccaaacacagcatcgatggcatcctgggcgacaaagggaaccgtctggatgagggctcagatgtggaatcagaacccgacctccccctgaagcgcaagcagcgccgcagtcggaccacgttcacagccgagcagctggaggagctggagaaggccttcgagaggacccactacccggacatctacacccgggaggagctggcacagaggaccaagctcacggaggcacgcgtccaggtctggttcagtaaccggcgtgcccgctggcgtaagcaggcaggagctaaccagctggccgccttcaaccaccttctgccgggaggtttcccacccaccggcatgcccacgctgccaccctaccagctgccggactctacctaccccaccaccaccatctcccaagatggaggcagcacagtacacaggccccagccccttccgccatcaaccatgcatcagggtgggctggctgcggccgctgcagcagcggacaccagctctgcctacggagcccgccacagcttctccagctactctgacagcttcatgaaccctggggctccctccaaccacatgaaccctgtcagcaatggcctgtctcctcaggtcatgagcatccttagcaacccgagtgccgtgcctccacagccccaggccgacttctccatctccccgctgcatggaggcctggactcggcttcctccatctcagccagctgcagccaacgggccgactccatcaagccaggagacagcttgcccacgtcccagtcttactgcccacccacctacagcaccactggctacagtgtggaccctgtggctggctaccagtacagccagtatggccaaactgctgttgattacctggccaaaaacgtgagcctgtccacacagcgccgtatgaagcttggggaacactccgctgtgctgggacttcttcctgtggaaacgggacaagcctactagggtccctggggcaacttgccccatccagtggcccagccaacccttcccaagccctgagtctcctcacctcagtccccttatccccctggggttgcaggagaccaagggaaaaaaaccctttcccttcctacaggaaaccctctggagacggaaaaccagtgtgccatctacccatgcttagtgacccagagtggccccttgccttcccctctttcttcagaggggttcctaggcatcctgcagtgacctccagctcacatccaccttctctgtatcgtggcctcggtcctgtctcagtgcagagattgaggctcaatttgaaccaagcacctagttatcagaagaaaatggtgccaaagacaaggccctggagtccttgacctctgagtcgtgggtgccctggctatgggtgtaggtggagcccatgggtgtcctcagtcacagagctgggagctctctctcgctcgcttggcatcaggactgcagcctctttcactggacactgagatgagtccccagggtgttcccaggggagaaagcaggtaacatcccagctttacctaggaatccagaggactttaggactgtccctatgcaccctgcagggcatcaggagaccaggaagggattctagcagagggtagggggcacagaggcagagctgattgcccatgggctatcccagaatgcctggtcctgaatctagcatcaggaggtgcaggattcctaggctgcaatctgacagaggcttgcccactgtgtcaggcctgggcagcccacagaacctgtcactctcctcaattggtaggagaagaggtcttgaggtgacaggaggcagaaggcaggctcagacagtcagagagcaccaagctttcaagtccgcacccctggggttcggcataccatcttgctggcagctggaaacctggttccctgaaagggggcctccatcctccagaatgtaaggctcttgatgccaccggatgcagagagccttctcgggccagacaaaattgctgctccaccccagagaagatgttccagccttcttggcatcttagaggaaggccatgtcgctgtccttttcagagtagcatatttttcagtgatggctgctcagtcaggaggcttctgtcgccttacaaagcacagtgcgctctgggcactgtttctaagccaccccatcccacccccacccccgccaccccggggacagaggaagatgctaaaagtcccagcaaagaggacaaagcacctttcttaagcactccagagtcttccttgtaccccgccctctcttagagctgggtcttttgagggaaacggattgctgagccctcccccccccccaatcctctcctctgtggagctgtttatcatcctctatttatcaaaatcgcatccatctttaccctctccttcgctatagcctacttctggatcaccctcatccagtgctggtaccccacagcactaaatccaggaaccctgggcttgatcacctgttgccacctgtacacatgaaaccacctgctggcccggcccatgtctcctgccctcagccagcaagacattcctagagagaggaactatgggcttaaaagccccaactgacttccttttgcctggggacctgaaccgacaagacaccagggacacttgtctacatgaacatgtgaccaatgtacaccgatttctcatctctagacctattatctgaagcctgtcccgggccatgactagaatggcttgtatctgtggtttagagaagtctaataataactgagggcaaactgactctctggtagcatggagcaccaggcggatggagctcaccagctctgtccaggtttcaaaggaggagactgttgggctcttcaaggtctggacaagaggaaagccacattgcccccttgggaacccaggttctccttttgaacttctcacagctgcaagcacccctttcaaagaccaaatgcatcctcctccacattccttgctccctggaggcctggctctggatacacctgagtcttcgttcacctactacactttaggagcaggaacttcaagcaggtgacatccacagggcccagtcccagccaagggagcaacattccaacgcttggaccaatcataatgatctgcccgtgagggtaaccgcaactagagacctgcttgggagaaaacaaaatgacttctcattccatgccatgcctctgaatgctcccccaagctgccatcttggtataaaatgggacttgtgttgtggggaaccccttgaccccaacaggttttcccaactgtctcatgcttttgtgaatctgtctgctttgatctgtaaaactcagccttgtttgggcagcttgtaatttcaacagtgaggcgacatcgattagatgagaggcaccaggcctctccgccgccgtccctctgtggccgtccctctggggttgagcagaacctagaagaaggccgatttccagtggccagactggaccagaaacagcccccaccccaatccctgtaaatagagtcaatagcaaaataagaggggcgccctccatgtcacctcaagtagctactggttcttctgtggaggcccctctgaactcattgtctggtagttgaaaatgtgatgttgtgctgtttgtttatagaacattggctttttatatataaatctatatacttaaaaacaaaaac

SEQ ID NO: 66 - protein, 503 aa, Mus musculus
MAALPGAVPRMMRPGPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEDDEEGDKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHSFSSYSDSFMNPGAPSNHMNPVSNGLSPQVMSILSNPSAVPPQPQADFSISPLHGGLDSASSISASCSQRADSIKPGDSLPTSQSYCPPTYSTTGYSVDPVAGYQYSQYGQTAVDYLAKNVSLSTQRRMKLGEHSAVLGLLPVETGQAY

SEQ ID NO: 67 - DNA, 7087 bp, Mus musculus
ggcacgagcctaggcggggggcgtcggcgctgggcccgccatggccgcgtccgagctctacaccaagtttgccagggtttggatccctgatcctgaggaagtgtggaaatcggcagagttgctcaaggattataagcctggagataaagtgctcctgcttcacctcgaggaagggaaggatttggaataccgtctagacccaaagaccggtgagctccctcacttacggaaccctgacatacttgttggagaaaatgacctcacagccctcagctaccttcacgagcccgctgtgctacataatctccgagttcgcttcatcgattccaaacttatttatacgtattgtggaatagttctggtagctataaatccctatgagcagctgcctatttatggagaagatattattaatgcctacagtggccagaacatgggtgacatggatcctcacatcttcgcagtagctgaagaggcttacaagcaaatggcaagggatgaacgaaatcagtccatcattgtaagtggagagtcaggcgcagggaagacggtctctgctaagtatgccatgcggtacttcgcaactgtaagtggctctgccagtgaggccaatgttgaggaaaaggtcttggcctccaaccccatcatggagtcaattggaaatgccaaaacaaccaggaatgataatagcagccggtttggaaaatatattgaaattggttttgacaagaggtacagaatcatcggtgccaatatgagaacttaccttttagagaaatccagagtggtgttccaggcagaagaggagagaaactaccatatcttctatcagctctgtgcttcggcaaagttacctgagtttaaaatgctgcggttaggaaatgcagatagttttcattacacgaagcaaggaggcagccctatgatagaaggagtagatgatgcgaaggagatggcgcacaccaggcaggcctgcactctgctgggaattagtgaatcttaccaaatgggaatttttcgaatacttgctggcattcttcacttaggcaatgttggatttgcatctcgggattcagacagctgcacaatacctcccaagcacgaacctcttaccatcttctgtgaccttatgggtgtggattatgaagagatgtgtcactggctctgccaccgaaagctggctactgccacagagacatacatcaagcccatctccaagctgcaggccacaaatgcccgagatgctttagcaaagcacatctatgcaaagctctttaactggattgttgaccacgtcaatcaggctctccattctgctgtcaagcagcactctttcatcggcgtgctggacatctatggatttgaaacatttgaaataaatagttttgaacagttctgcataaattatgcaaatgaaaaactacaacaacaattcaacatgcatgtcttcaaattggaacaggaggaatatatgaaggaacagattccatggacacttatagatttctatgataatcagccttgtatcaatcttatagaatctaaactgggaattctcgatttgctggatgaggaatgtaagatgcctaaaggcacagatgacacatgggcccaaaaactgtacaacacacatttgaacaaatgtgccctctttgagaagccccgcatgtcaaacaaagctttcatcatcaaacattttgctgacaaagtggagtaccagtgtgaaggttttcttgaaaagaataaagatactgtttttgaagaacaaattaaagtccttaagtcaagcaagtttaagatgctaccagagctatttcaagatgatgagaaggccatcagtcccacctctgccacctcctcagggcgcactcctctcactcgagttcctgtaaagcccaccaagggccgacctggccagactgccaaagagcacaagaagacagtgggacatcagtttcgaaactcccttcacctgcttatggaaacccttaatgccactactcctcactatgtacgctgtattaagcctaatgatttcaagtttccattcacatttgatgagaagagggcagtgcagcagctaagagcatgtggtgtcctggagaccatccggatcagcgcacgagggtttccctcacggtggacttaccaagagtttttcagccggtaccgggtcctaatgaagcaaaaagatgtgctgggagatagaaagcaaacgtgcaagaatgtattagagaaactaatattggacaaggataaataccagtttggtaagacaaagatctttttccgtgctggtcaagtggcctatcttgaaaaattgagggctgacaaacttcgggctgcctgcatccggatccagaagaccattcgtgggtggcttctaaggaagagatacctgtgtatgcagagggcagccatcacagtgcagcgatacgtgcggggctatcaggctcgatgctatgctaagtttctgcgcagaaccaaggcagcaaccaccattcaaaagtactggcgcatgtatgtggtccgcaggaggtacaagattagacgagctgccacgattgttattcagtcttacttgagaggctacttgacaagaaataggtatcgcaagatactccgtgaatacaaagcagtcatcattcagaaacgtgtccgtggctggctggcccgtacacattataagaggaccatgaaagccatcgtctaccttcagtgctgcttccggcggatgatggccaagcgtgacgtgaagaaactcaaaattgaggctcgctctgtggaacgctacaagaagctccatattggcatggagaacaagattatgcagctgcagcgcaaagtggatgagcagaataaagactacaaatgcctcatggagaaactgaccaatctggaaggagtatacaactctgagactgaaaaactacgaaatgatgtagaacgtcttcagctaagtgaagaggaagctaaggttgccactgggcgggtccttagtctgcaggaagaaattgcaaaactccgaaaagacctggaacaaactcgatcagagaaaaagtctattgaagaaagagcagataaatacaaacaagaaactgaccagctggtgtcaaacttgaaggaagaaaatactttgctgaagcaggaaaaggagaccctcaaccaccgcattgtggagcaggcgaaggagatgacagaaactatggagaggaagttagtagaagagacaaaacaactggagcttgacctgaatgatgagaggctgaggtatcagaaccttctgaatgagttcagtcgcctggaggagcgctatgatgacctcaaggaagagatgaccctgatgctgaatgtgcctaagccaggacacaagagaacagactctacccacagcagtaatgagtctgaatacaccttcagctctgagtttgcagaaactgaagacattgcaccaagaacagaggagccaattgagaagaaggtgcctctggatatgtcactattccttaagctccagaagcgtgtcacagagctggaacaggagaagcagctgatgcaggatgagctggaccgcaaggaggagcaggtgttccgcagcaaggcaaaggaagaagaaaggccacagataagaggagctgaactagagtatgagtctctcaagcgtcaagaactggagtcagaaaacaaaaaactgaaaaacgagctgaatgagttgcgcaaagccctcagtgagaaaagtgccccagaagtgactgcgccaggtgcgcctgcttaccgagtcctcatggaacagctgacctccgtgagcgaggagctcgacgtgcgcaaggaggaagtcctcatcttgaggtcgcagctggtgagccaaaaagaagccatccaacccaaggatgacaagaatacaatgacagattccacaattcttttagaagatgtacagaaaatgaaagacaaaggtgaaatagcacaagcatatattggtttgaaagaaacaaacaggctcttagaatcccagctacagtcacagaaaagaagccatgagaatgaggctgaggccctccgtggggagatccagagcctaaaggaagaaaacaaccggcaacagcagctgctggcccagaacctgcagctgccccctgaggcccgcattgaggccagcctgcagcatgagatcacccggctgaccaatgaaaacctgtattttgaggaattatatgcagatgaccctaagaagtatcaatcctatcggatttcactttacaaaaggatgattgatctgatggaacaacttgaaaagcaggataaaactgtccggaaactgaagaaacaactgaaagtctttgccaaaaaaattggtgaactagaagtggggcagatggagaacatatctccaggacagatcatcgatgagcctatccggccagtcaacattccccggaaagaaaaggatttccaaggaatgctggagtacaaaagggaggatgagcagaagctagtgaagaacctgattctagaactaaagccacgtggtgtggctgtcaatctgattccagggttaccagcatatatcttgtttatgtgtgtacgacatgctgactatctgaacgatgatcagaaagtaagatcattgctgacatcaacaattaacagcatcaaaaaagtcttgaagaaaagaggtgacgattttgaaactgtctccttctggctctctaacacatgtcgatttttgcactgtttgaagcaatatagtggagaggagggctttatgaagcacaacacgtctcgccagaatgagcactgcctcaccaattttgaccttgctgaatatcggcaagtactgagtgacttggccattcagatctatcagcagcttgtgagggtgttagagaacattcttcagccaatgatagtctcaggtatgctagagcatgaaacaattcagggcgtatctggggtgaagcccacagggctcagaaaacgaacctccagtatcgcagatgagggcacctacacactggactccattcttcggcagctcaactccttccactcagtcatgtgtcagcatggcatggacccggagctaatcaagcaggtagtcaaacagatgttctacattgtgggcgccatcaccctaaacaacctcctcctgcgcaaggacatgtgctcctggagcaagggcatgcagatcaggtacaatgtcagtcaactggaagaatggctacgtgacaagaatctaatgaacagtggggccaaggagactctggaacctcttatccaggccgctcagcttttgcaagtgaaaaagaaaactgacgatgacgcagaagccatctgttccatgtgcaacgccctgaccacggcccagattgtcaaagtgttgaatctgtacacaccggttaatgagtttgaagaaagggtctcagtttcatttatccgcactatacagatgcggttacgagacaggaaagactctccacagctgctcatggatgcaaaacacatctttcctgtcacttttccctttaacccatcctccctagccctagaaaccatccagatcccagccagcctgggcctgggcttcatcgcacgggtctgaagtgctgtccaagcaaacgtagaaaacgcatttctcctcagaatatgaacagttgtttccagtgagctactgaaaatgcatttttaaagaaaaagtactgattatctctaaaggaagatgttaactggaaatccacccctccccccagaatcctttcgattgaccaacctggaggacacccctcgtactgttacctttaaacaacatttattctttatcagttggaaacctgagtttacatacagctgaaaggtggcagggaggagtgaaaggaaggataaaggggtgtgtcttcagctgactgcttttagtttaaatctttagtgctgcatttaagtggcatacaaaatacaatcccatatgtatgaactgttgtgaacaaggcacaaagaactgaagaaaatcactgaatacatatcatagcagctttggagccaatcatgttgatattgtcaaactggatcacaaaaataaactggcaacattaaagtaactggccaagtaacttccttctctgtgtccctacggcatagcgatgtgaaagctgctgtttgctaggccgtgtagatgcccgtttgcactgtagcttactgagcatcttaagtggctgctgtgtgctgtctcctgacctaagtgaaagtcttactaggactataaactacgcttctctttcatcagctctgtttacaagtcaaccagatcagttctaaccggattatatacagttatccacaggttcatcagcgcgagtagcagacgctggggaagtagagttagacagagtggctgttgatgtcattgctgatcggattattacattgataagtcatctgtgtggatatcacatcatggtatagagttgtgtaaagttacttggttttagttcccaaatctgattcttgccatgcatatattcaaccttgacaagttttctttttaatttattaggtgttagtttcagtcactatctggaaaggactgtcagaatatttatgactaagatcttgaaactgtttatcctataagttattcaaaataggcatataaataaatcattataattcagggggcttaggtgttgcaacctctataaatgttctcagataccactggatactttaaaaatatcatattataaacacattaagaagacgtatgtaagaagtataggagattattaaactttatagaggatttggatcagtgagatccaggttcagtatgtcttcattatgaaatcctagttaagcatattctccgtttttttttcctggaagtcttatatagtaaatcctttgggttttgcaagagcaaattcttagctttcattcctctggttcttaattacaatcatcaatttgtatagtttaactgtaagatggtctataaccgtgacatttaaatatgtgcaatattattaatgagagctaaatggtttcaagtcaatgataaatactgttattaaacaacaattattgggaattc

SEQ ID NO: 68 - protein, 1853 aa, Mus musculus
MAASELYTKFARVWIPDPEEVWKSAELLKDYKPGDKVLLLHLEEGKDLEYRLDPKTGELPHLRNPDILVGENDLTALSYLHEPAVLHNLRVRFIDSKLIYTYCGIVLVAINPYEQLPIYGEDIINAYSGQNMGDMDPHIFAVAEEAYKQMARDERNQSIIVSGESGAGKTVSAKYAMRYFATVSGSASEANVEEKVLASNPIMESIGNAKTTRNDNSSRFGKYIEIGFDKRYRIIGANMRTYLLEKSRVVFQAEEERNYHIFYQLCASAKLPEFKMLRLGNADSFHYTKQGGSPMIEGVDDAKEMAHTRQACTLLGISESYQMGIFRILAGILHLGNVGFASRDSDSCTIPPKHEPLTIFCDLMGVDYEEMCHWLCHRKLATATETYIKPISKLQATNARDALAKHIYAKLFNWIVDHVNQALHSAVKQHSFIGVLDIYGFETFEINSFEQFCINYANEKLQQQFNMHVFKLEQEEYMKEQIPWTLIDFYDNQPCINLIESKLGILDLLDEECKMPKGTDDTWAQKLYNTHLNKCALFEKPRMSNKAFIIKHFADKVEYQCEGFLEKNKDTVFEEQIKVLKSSKFKMLPELFQDDEKAISPTSATSSGRTPLTRVPVKPTKGRPGQTAKEHKKTVGHQFRNSLHLLMETLNATTPHYVRCIKPNDFKFPFTFDEKRAVQQLRACGVLETIRISARGFPSRWTYQEFFSRYRVLMKQKDVLGDRKQTCKNVLEKLILDKDKYQFGKTKIFFRAGQVAYLEKLRADKLRAACIRIQKTIRGWLLRKRYLCMQRAAITVQRYVRGYQARCYAKFLRRTKAATTIQKYWRMYVVRRRYKIRRAATIVIQSYLRGYLTRNRYRKILREYKAVIIQKRVRGWLARTHYKRTMKAIVYLQCCFRRMMAKRDVKKLKIEARSVERYKKLHIGMENKIMQLQRKVDEQNKDYKCLMEKLTNLEGVYNSETEKLRNDVERLQLSEEEAKVATGRVLSLQEEIAKLRKDLEQTRSEKKSIEERADKYKQETDQLVSNLKEENTLLKQEKETLNHRIVEQAKEMTETMERKLVEETKQLELDLNDERLRYQNLLNEFSRLEERYDDLKEEMTLMLNVPKPGHKRTDSTHSSNESEYTFSSEFAETEDIAPRTEEPIEKKVPLDMSLFLKLQKRVTELEQEKQLMQDELDRKEEQVFRSKAKEEERPQIRGAELEYESLKRQELESENKKLKNELNELRKALSEKSAPEVTAPGAPAYRVLMEQLTSVSEELDVRKEEVLILRSQLVSQKEAIQPKDDKNTMTDSTILLEDVQKMKDKGEIAQAYIGLKETNRLLESQLQSQKRSHENEAEALRGEIQSLKEENNRQQQLLAQNLQLPPEARIEASLQHEITRLTNENLYFEELYADDPKKYQSYRISLYKRMIDLMEQLEKQDKTVRKLKKQLKVFAKKIGELEVGQMENISPGQIIDEPIRPVNIPRKEKDFQGMLEYKREDEQKLVKNLILELKPRGVAVNLIPGLPAYILFMCVRHADYLNDDQKVRSLLTSTINSIKKVLKKRGDDFETVSFWLSNTCRFLHCLKQYSGEEGFMKHNTSRQNEHCLTNFDLAEYRQVLSDLAIQIYQQLVRVLENILQPMIVSGMLEHETIQGVSGVKPTGLRKRTSSIADEGTYTLDSILRQLNSFHSVMCQHGMDPELIKQVVKQMFYIVGAITLNNLLLRKDMCSWSKGMQIRYNVSQLEEWLRDKNLMNSGAKETLEPLIQAAQLLQVKKKTDDDAEAICSMCNALTTAQIVKVLNLYTPVNEFEERVSVSFIRTIQMRLRDRKDSPQLLMDAKHIFPVTFPFNPSSLALETIQIPASLGLGFIARV

SEQ ID NO: 69 - DNA, 2241 bp, Homo sapiens
ggcgctctctgtcccgctcggagctgctcggcgccccagctgcccgccccgccggccgctcctgcccgcggcgcagatggctgtgcgcggcgccaacaccttgacgtccttctccatccaggcgatcctcaacaagaaagaggagcgcggcgggctggccgcgccagaggggcgcccggcgcccgggggcacagcggcatcggtggccgcggctcccgctgtctgctgttggcggctctttggggagagggacgcgggcgcgttggggggcgccgaggactctctgctggcgtctcctgccggtaccagaacagctgcggggcggactgcggagagcccggaaggctgggactcggactccgcgctcagcgaggagaacgagagcaggcggcgctgcgcggacgcgcggggggccagcggggccggccttgcggggggatccttgagcctcggccagccggtctgtgagctggccgcttccaaagacctagaggaggaagccgcgggccggagcgacagcgagatgtccgccagcgtctcaggcgaccgcagcccaaggaccgaggacgacggtgttggccccagaggtgcacacgtgtccgcgctgtgcagcggggccggcggcgggggcggcagcgggccggcaggcgtcgcggaggaggaggaggagccggcggcgcccaagccacgcaagaagcgctcgcgggccgctttctcccacgcgcaggtcttcgagctggagcgccgctttaaccaccagcgctacctgtccgggcccgagcgcgcagacctggccgcgtcgctgaagctcaccgagacgcaggtgaaaatctggttccagaaccgtcgctacaagacaaagcgccggcagatggcagccgacctgctggcctcggcgcccgccgccaagaaggtggccgtaaaggtgctggtgcgcgacgaccagagacaatacctgcccggcgaagtgctgcggccaccctcgcttctgccactgcagccctcctactattacccgtactactgcctcccaggctgggcgctctccacctgcgcagctgccgcaggcacccagtgaacccgcttgggctgaggcagcgagtgattcccgcgctccggctccggaccggcgctgacagctgtaggctgtagcctgcacggggcgccccgccaaggaggcacctggaggtgaaacccagctccagctcccgttagccaggacttgtcccctggcagctgggctgagtctgccctgagggggcgcctttttctaatttgaacagaggcaccctatggcctaggggccctgatcgcccacctgcctggaagcccctgggctctatttattatcatgacaatgttggaattaaattttgattcgaatatgtctgcctgggggtggggttttccctgagcggcaactcctggagaccacatagcctgaatcctcagaatttcaggcctgctgggagctttctgcactaggccacactagttcatggtatccatgctaccaatctatgtgtatctacatatcttttatttttggaaattgcatttgtaaccaaggggtgcgaaaccctggcagtcccaggcagcaccaggccaggggttgatttgaaacgtgaaggattgggttttcaggccctctgctccacccctcctgtgtgtcagagctagggtgggggtgcccgattcgggtgctgaatgtaaggaggggagcctccaagtgtggtgcaagccgggggtctccacatcttccttctctgaagtccaggtacctgcacaagcaggaagcgcctgggagtcccggaaggaggagagcgcacacccaggcagccctctgcggaaactttccttggtttctttttatttgtgtaaaggaggttaagacgtgtcgcacttttcagttgtttgtattcaaatgacgattatttttctactcaatgtgaatatccctggccagcctttccacggcgcccaccgcagtgccgctgcctggccctcagtgtctaccttctgccctctgcgactccagtgctctggcccgggactcccctatccgcccctcacttacccttaaacaggtgatcccacctgtcttgtcaacctcgccgcttttcgcctccttaatggcactgtgcactcaactagagtattaactgtaaaaagatttgtgaagtttggaagctctattcgctgtattttttctttaatttataaacttttagtttaacatgc

SEQ ID NO: 70 - protein, 333 aa, Homo sapiens
MAVRGANTLTSFSIQAILNKKEERGGLAAPEGRPAPGGTAASVAAAPAVCCWRLFGERDAGALGGAEDSLLASPAGTRTAAGRTAESPEGWDSDSALSEENESRRRCADARGASGAGLAGGSLSLGQPVCELAASKDLEEEAAGRSDSEMSASVSGDRSPRTEDDGVGPRGAHVSALCSGAGGGGGSGPAGVAEEEEEPAAPKPRKKRSRAAFSHAQVFELERRFNHQRYLSGPERADLAASLKLTETQVKIWFQNRRYKTKRRQMAADLLASAPAAKKVAVKVLVRDDQRQYLPGEVLRPPSLLPLQPSYYYPYYCLPGWALSTCAAAAGTQ

SEQ ID NO: 71 - DNA, 3963 bp, Homo sapiens
ggagagccgaaagcggagctcgaaactgactggaaacttcagtggcgcggagactcgccagtttcaaccccggaaacttttctttgcaggaggagaagagaaggggtgcaagcgcccccacttttgctctttttcctcccctcctcctcctctccaattcgcctccccccacttggagcgggcagctgtgaactggccaccccgcgccttcctaagtgctcgccgcggtagccggccgacgcgccagcttccccgggagccgcttgctccgcatccgggcagccgaggggagaggagcccgcgcctcgagtccccgagccgccgcggcttctcgcctttcccggccaccagccccctgccccgggcccgcgtatgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttgaggaggcctcccacgaagggcgaagatggccgagatgatcctaaaaataaccgaagaaagagaggaccaaccagaattccctttggacatttgtgtttttttgtttttttattttgttttgttttttcttcttcttcttcttccttaaagacatttaagctaaaggcaactcgtacccaaatttccaagacacaaacatgacctatccaagcgcattacccacttgtggccaatcagtggccaggccaaccttggctaaatggagcagcgaaatcaacgagaaactggactttttaaaccctcttcagagcaagcgtggaggatgatggagaatcgtgtgatcagtgtgctaaatctctctgcctgtttggactttgtaattatttttttagcagtaattaaagaaaaaagtcctctgtgaggaatattctctattttaaatatttttagtatgtactgtgtatgattcattaccattttgaggggatttatacatatttttagataaaattaaatgctcttatttttccaacagctaaactactcttagttgaacagtgtgccctagcttttcttgcaaccagagtatttttgtacagatttgctttctcttacaaaaagaaaaaaaaaatcctgttgtattaacatttaaaaacagaattgtgttatgtgatcagttttgggggttaactttgcttaattcctcaggctttgcgatttaaggaggagctgccttaaaaaaaaataaaggccttattttgcaattatgggagtaaacaatagtctagagaagcatttggtaagctttatcatatatatattttttaaagaagagaaaaacaccttgagccttaaaacggtgctgctgggaaacatttgcactcttttagtgcatttcctcctgcctttgcttgttcactgcagtcttaagaaagaggtaaaaggcaagcaaaggagatgaaatctgttctgggaatgtttcagcagccaataagtgcccgagcacactgcccccggttgcctgcctgggccccatgtggaaggcagatgcctgctcgctctgtcacctgtgcctctcagaacaccagcagttaaccttcaagacattccacttgctaaaattatttattttgtaaggagaggttttaattaaaacaaaaaaaaattcttttttttttttttttccaattttaccttctttaaaataggttgttggagctttcctcaaagggtatggtcatctgttgttaaattatgttcttaactgtaaccagtttttttttatttatctctttaatctttttttattattaaaagcaagtttctttgtattcctcaccctagatttgtataaatgcctttttgtccatcccttttttctttgttgtttttgttgaaaacaaactggaaacttgtttctttttttgtataaatgagagattgcaaatgtagtgtatcactgagtcatttgcagtgttttctgccacagacctttgggctgccttatattgtgtgtgtgtgtgggtgtgtgtgtgttttgacacaaaaacaatgcaagcatgtgtcatccatatttctctgcatcttctcttggagtgagggaggctacctggaggggatcagcccactgacagaccttaatcttaattactgctgtggctagagagtttgaggattgctttttaaaaaagacagcaaacttttttttttatttaaaaaaagatatattaacagttttagaagtcagtagaataaaatcttaaagcactcataatatggcatccttcaatttctgtataaaagcagatctttttaaaaagatacttctgtaacttaagaaacctggcatttaaatcatattttgtctttaggtaaaagctttggtttgtgttcgtgttttgtttgtttcacttgtttccctcccagccccaaaccttttgttctctccgtgaaacttacctttccctttttctttctcttttttttttttgtatattattgtttacaataaatatacattgcattaaaaagaaaaaaaaaaaaaaaaaaaaaaaaaaaaaaa

SEQ ID NO: 72 - protein, 509 aa, Homo sapiens
MNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQPGKADLKREGRPLPEGGRQPPIDFRDVDIGELSSDVISNIETFDVNEFDQYLPPNGHPGVPATHGQVTYTGSYGISSTAATPASAGHVWMSKQQAPPPPPQQPPQAPPAPQAPPQPQAAPPQQPAAPPQQPQAHTLTTLSSEPGQSQRTHIKTEQLSPSHYSEQQQHSPQQIAYSPFNLPHYSPSYPPITRSQYDYTDHQNSSSYYSHAAGQGTGLYSTFTYMNPAQRPMYTPIADTSGVPSIPQTHSPQHWEQPVYTQLTRP

SEQ ID NO: 73 - protein, 14 aa, Artificial Sequence
  The sequence has been designed and synthesized.
GKPIPNPLLGLDST

(19) United States
(12) Patent Application Publication
(10) Pub. No.: US 2014/0004047 A1
(43) Pub. Date: Jan. 2, 2014

(54) Methods, compositions and kits for modulating trans-differentiation of muscle satellite cells

(71) Applicant: Tufts University, Boston, MA (US)

(72) Inventors: Li Zeng, Framingham, MA (US); Dana M. Cairns, Quincy, MA (US)

(21) Appl. No.: 13/939,367

(22) Filed: Jul. 11, 2013

Related U.S. Application Data
(63) Continuation of application No. PCT/US12/20933, filed on Jan. 11, 2012.
(60) Provisional application No. 61/431,708, filed on Jan. 11, 2011.

(51) Int. Cl. C07K 14/475; G01N 33/68; C12Q 1/68

(52) U.S. Cl. CPC C07K 14/475; C12Q 1/6883; G01N 33/6887
USPC 424/9.2; 514/16.5; 435/34; 435/6.13; 435/6.12; 435/7.1; 435/7.4; 530/350; 536/23.5; 536/24.5

(57) ABSTRACT

Compositions, methods and kits are provided for modulating the trans-differentiation of cells for example muscle satellite cells. The compositions, methods and kits include a modulator of trans-differentiation of the muscle satellite cells selected from the group of: a transcription factor, a nucleic acid sequence or vector encoding expression of the transcription factor, and an agent that binds to the transcription factor. The transcription factor is selected for example from a homeodomain class transcription factor such as Nkx3.2 and a TATA binding protein class transcription factor such as Sox9, and includes at least one nucleotide binding-domain, so that the transcription factor modulates the process of trans-differentiation of the cells or tissue to form a phenotype selected from: cartilage, muscle, and bone.

### RELATED APPLICATION

This application is a U.S. continuation of international application number PCT/US12/20933 filed Jan. 11, 2012 which claims the benefit of and priority to U.S. provisional application Ser. No. 61/431,708 filed Jan. 11, 2011, titled “Methods, compositions and kits for modulating trans-differentiation of muscle satellite cells”, inventors Li Zeng and Dana M. Cairns, each of which is hereby incorporated herein by reference in their entireties.

### GOVERNMENT SUPPORT

This invention was made with government support under grant numbers 1R03AR054611 and 1R01AR059106-01A1 awarded by the National Institutes of Health, and CBET-0966920 and CBET-0966920 awarded by the National Science Foundation. The government has certain rights in the invention.

### TECHNICAL FIELD

Compositions, methods, and kits for modulating trans-differentiation of muscle satellite cells to chondrocytes or bone, and methods for identifying a modulator of trans-differentiation of muscle satellite cells are provided herein.

### BACKGROUND

Skeletal muscle includes highly differentiated contractile fibers that perform the actions of the body, and muscle satellite cells that differentiate into myocytes to form mature contractile fibers and regenerate into new muscle satellite cells. Muscle satellite cells, also referred to muscle stem cells, differentiate into cells having alternative and distinct phenotypes such as fat and bone that in unfortunate cases cause painful masses and abnormalities in soft tissue of subjects.

Bone morphogenic protein (BMP) signaling plays a role in forming cartilage and bone. The mechanisms that lie downstream of BMP signaling that are responsible for muscle differentiating to cartilage or bone remain unidentified and unclear. There is a need for methods and compositions for modulating muscle satellite cells for regenerating muscle in subjects and preventing abnormal formation of bone in soft tissue.

### SUMMARY

An aspect of the invention provides a pharmaceutical composition for modulating trans-differentiation of muscle satellite including a modulator of trans-differentiation of the muscle satellite cells selected from the group of: a transcription factor, a nucleic acid encoding expression of the transcription factor, and an agent that binds to the transcription factor, such that the transcription factor is selected from a homeodomain class transcription factor and a TATA binding protein class transcription factor and comprises at least one nucleotide binding-domain, such that the transcription factor modulates trans-differentiation of the muscle satellite cells to chondrocytes and bone.

The transcription factor in an embodiment of the pharmaceutical composition includes an NKX protein or a portion thereof. For example, the NKX protein is a Nkx3.2 protein or a Nkx3.2 protein having a deleted or altered terminal domain. In an embodiment of the pharmaceutical composition, the terminal domain includes a carboxy-end or amino end. In various embodiments of the pharmaceutical composition, the NKX protein optionally further includes at least one sequence selected from the group of: SEQ ID NO: 41, SEQ ID NO: 42, SEQ ID NO: 59, SEQ ID NO: 60, SEQ ID NO: 69, SEQ ID NO: 70, and substantially identical.

The sequence listing material in computer readable form ASCII text file (209 kilobytes) created Jan. 11, 2012 entitled “SEQ_ID_{—}01122012”, containing sequence listings numbers 1-73, has been electronically filed herewith and is incorporated by reference herein in its entirety.

As used herein, the term “substantially identical” means that the sequence has at least about 60% identity, at least about 65%, at least about 70% identity, at least about 75%, at least about 80% identity, at least about 85%, at least about 90%, at least about 95%, or at least about 99% identity or homology to an nucleic acid sequence or an amino sequence herein for the modulator or the transcription factor.

For example, the gene encoding the Nkx3.2 protein or portion thereof includes at least one nucleic acid sequence selected from the group of: SEQ ID NO: 41, SEQ ID NO: 59, SEQ ID NO: 69, and substantially identical. In various embodiments of the pharmaceutical composition, the Nkx3.2 protein includes at least one amino acid sequence selected from the group of SEQ ID NO: 42, SEQ ID NO: 60, SEQ ID NO: 70, and substantially identical. In related embodiments of the pharmaceutical composition, the Nkx3.2 protein includes an amino acid sequence at least about 30%, about 35%, about 40%, about 45%, about 50%, about 55%, about 60%, about 65%, about 70%, about 75%, about 80%, about 85%, about 90%, about 95%, or about 99% identical to at least one selected from: SEQ ID NO: 42, SEQ ID NO: 60, SEQ ID NO: 70, and a portion thereof.

The transcription factor in an embodiment of the pharmaceutical composition includes a Sox protein or a portion thereof, for example the Sox protein is a Sox9. In various embodiments of the pharmaceutical composition, the Sox protein optionally further includes at least one sequence selected from the group of: SEQ ID NO: 43, SEQ ID NO: 44, SEQ ID NO: 61, SEQ ID NO: 62, SEQ ID NO: 71, SEQ ID NO: 72, and substantially identical. In various embodiments of the pharmaceutical composition, the gene encoding the Sox9 protein or portion thereof includes at least one sequence selected from the group of: SEQ ID NO: 43, SEQ ID NO: 61, and SEQ ID NO: 71. In various embodiments of the pharmaceutical composition, the Sox9 includes at least one amino acid sequence selected from the group of: SEQ ID NO: 44, SEQ ID NO: 62, SEQ ID NO: 72, and substantially identical. In related embodiments of the pharmaceutical composition, the Sox9 protein includes an amino acid sequence having at least about 30%, about 35%, about 40%, about 45%, about 50%, about 55%, about 60%, about 65%, about 70%, about 75%, about 80%, about 85%, about 90%, about 95%, or about 99% identical to SEQ ID NO: 44, SEQ ID NO: 62, SEQ ID NO: 72, or a portion thereof.

The pharmaceutical composition in an embodiment includes a fusion protein of the transcription factor. For example, the fusion protein includes at least one of: an Nkx protein or portion thereof, a Sox protein or portion thereof, and a tag. For example, the tag includes SEQ ID NO: 73. In various embodiments of the pharmaceutical composition, the tag includes at least one of: an antibody epitope, including a polypeptide, sugar or DNA molecule. In an embodiment, the pharmaceutical composition further comprises a detectable marker.

In an embodiment of the pharmaceutical composition, the modulator alleviates or reduces a symptom of a disease or a disorder, for example the disease or the disorder is selected from the group of: heterotopic ossification; edema; formation of a tissue mass for example the mass comprises cartilaginous material; joint or muscle stiffness; joint or muscle pain; and arthritis.

The transcription factor or the agent in an embodiment of the pharmaceutical composition improves fracture healing for example by stimulating formation of bone, cartilage or muscle at a site of a fracture or adjacent to the fracture. In an embodiment of the pharmaceutical composition, the transcription factor includes an tag, for example an epitopic tag such as

The agent that binds the transcription factor in various embodiments of the pharmaceutical composition includes a transcription repressor. For example, the transcription repressor comprises a protein that negatively modulates the nucleic acid that encodes Nkx3.2 or Sox9.

An embodiment of the pharmaceutical composition provides the agent that binds to the transcription factor as including an siRNA that negatively modulates a nucleic acid that encodes the transcription factor, for example, the siRNA negatively modulates at least one of: Nkx3.2, Sox9, Pax3, Pax7, and myosin heavy chain. Alternatively, the agent that binds to the transcription factor includes an antibody or antibody fragment that negatively modulates a nucleic acid that encodes the transcription factor, or that binds directly to the transcription factor. For example, the antibody or the antibody fragment includes at least one of: a recombinant antibody, a Fv, a Fab, a Fab′, a F(ab′)₂, and a Fe.

The pharmaceutical composition is effective in one embodiment for increasing formation of cartilage or bone in a subject, for example increasing formation in the subject having a deficiency, defect, or fracture of the cartilage or the bone. In various embodiments, the pharmaceutical composition is optionally compound to be administered by at least one route of administration such as: topical, ocular, nasal, bucal, oral, rectal, parenteral, intracisternal, intravaginal, intraperitoneal, intra-bone, intra-cartilaginous, and intra-muscular. In various embodiments of the pharmaceutical composition, the modulator is compounded with a pharmaceutically acceptable buffer or carrier.

In general, the modulator is an active agent for modulation of trans-differentiation of muscle satellite by controlling differentiation pathways and expression of phenotype markers. Additionally or alternatively, the pharmaceutical composition is effective for increasing rate of healing a defect or an injury of a tissue such as bone, muscle, or cartilage.

An aspect of the invention provides a method for modulating trans-differentiation of muscle satellite cells of a subject, the method including: engineering a modulator of trans-differentiation of the muscle satellite cells, such that the modulator is selected from the group of a transcription factor, a nucleic acid sequence encoding expression of the transcription factor, and an agent that binds to the transcription factor, such that the transcription factor is selected from a homeodomain class transcription factor and a TATA binding protein class transcription factor, and includes at least one nucleotide binding-domain; contacting cells with the modulator; and, measuring an amount of at least one phenotype selected from chondrocyte, muscle, or bone in comparison to cells not so contacted and otherwise identical, such that an increase or a decrease in the phenotype in the cells compared to the cells not so contacted is an indication of modulation of the muscle satellite cells. In various embodiments, the cells are a plurality of muscle satellite cells or a plurality of cells adjacent to the muscle satellite cells.

In various embodiments of the method, the cells include living cells. In various embodiments of the method, the cells include at least one cell type selected from the group consisting of: epithelial cells, hematopoietic cells, stem cells, satellite cells, spleen cells, kidney cells, pancreas cells, liver cells, neuron cells, bone cells, muscle cells, adipose cells, cartilage cells, glial cells, smooth or striated muscle cells, sperm cells, heart cells, lung cells, ocular cells, bone marrow cells, fetal cord blood cells, progenitor cells, tumor cells, peripheral blood mononuclear cells, leukocyte cells, and lymphocyte cells.

An embodiment of the method, further includes engineering the modulator includes expressing in the cells a gene encoding an NKX protein or a portion thereof, for example an NKX family of homeodomain-containing transcription factors such as NK3 homeobox 2 (Nkx3.2). In an embodiment of the method, engineering the modulator comprises mutating a gene encoding an Nkx3.2 protein including deleting or modifying a portion of the gene encoding a carboxy-terminal (C-terminal) domain of the protein. For example, the method involves deleting or modifying the last 20 amino acids, 30 amino acids, 40 amino acids, 50 amino acids, or 60 amino acids of the C-terminal domain. In an embodiment of the method, the transcription factor includes a tag. In an embodiment of the method, the nucleic acid sequence encoding the transcription factor includes a signal for effectively expressing the transcription factor.

In an embodiment of the method, the transcription factor includes an NKX protein or a portion thereof. In various embodiments, the NKX protein optionally further includes at least one sequence selected from the group of: SEQ ID NO: 41, SEQ ID NO: 42, SEQ ID NO: 59, SEQ ID NO: 60, SEQ ID NO: 69, SEQ ID NO: 70, and substantially identical. For example, the NKX protein is derived from human, mouse, pig, or chicken.

For example, the NKX protein is derived from human, dog, cat, mouse, pig, or chicken.

In various embodiments of the method, the nucleic acid sequence encoding expression of the NKX protein, for example a Nkx3.2 protein, is selected from the group of: SEQ ID NO: 41, SEQ ID NO: 59, SEQ ID NO: 69, and substantially identical. In various embodiments of the method, the NKX protein comprises an amino acid sequence selected from the group of: SEQ ID NO: 42, SEQ ID NO: 60, SEQ ID NO: 70, and substantially identical.

In an embodiment of the method, the transcription factor includes a Sox protein or a portion thereof. In various embodiments, the Sox protein optionally further includes at least one sequence selected from the group of: SEQ ID NO: 43, SEQ ID NO: 44, SEQ ID NO: 61, SEQ ID NO: 62, SEQ ID NO: 71, SEQ ID NO: 72, and substantially identical. In various embodiments of the method, the nucleic acid sequence encoding expression of the Sox protein, for example a Sox9 protein, is selected from the group of: SEQ ID NO: 43, SEQ ID NO: 61, SEQ ID NO: 71, and substantially identical. In various embodiments of the method, the Sox protein comprises an amino acid sequence selected from the group of: SEQ ID NO: 44, SEQ ID NO: 62, SEQ ID NO: 72, and substantially identical.

An embodiment of the method further includes engineering the modulator includes expressing in the cells a gene encoding a Sox protein or a portion thereof. In an embodiment of the method, engineering the modulator includes expressing in the cells a gene encoding a Sox protein, for example a Sox9 protein. In an embodiment of the method, engineering the Sox9 protein includes constructing a Sox9 gene having a mutated high mobility group (HMG) box that has an altered ability to bind to the minor groove in DNA. For example, the Sox9 gene is engineered to decrease muscle formation and to increase cartilage formation in the cells.

In alternative embodiments of the method, engineering the modulator involves constructing a nucleic acid vector carrying the gene encoding the transcription factor; or a viral vector carrying a gene encoding the transcription factor. A related embodiment of the method includes engineering by constructing or synthesizing a viral vector recombinantly linked to the nucleotide sequence encoding the transcription factor. In various embodiments of the method, the vector is at least one selected from a retrovirus, an adenovirus, an adeno-associated virus, a herpesvirus, a poxvirus, and a lentivirus. For example, the virus is derived from a mammalian subject such as a human, a mouse, or a pig. In an embodiment, engineering the virus is derived from an avian species such as a chicken.

An embodiment of the method further includes engineering the transcription factor includes expressing a fusion protein in the cells. For example, the method involves engineering the fusion protein to include joining of two or more genes having a nucleic acid sequence which encodes a Nkx3.2 protein and a VP16 transcriptional domain. Contacting the cells with the modulator further includes, in alternative embodiments at least one route of administration selected from the group of: topical, ocular, nasal, bucal, oral, rectal, parenteral, intracisternal, intravaginal, and intraperitoneal. In an embodiment of the method, contacting the cells involves administering, for example by injecting, by at least one route selected from the group of: intramuscular, intra-cartilaginous, intra-bone, subcutaneous, and intravenous.

An embodiment of the method further includes contacting the cells or contacting a tissue in situ or in vivo. Alternatively, contacting the cells involves contacting a cell culture or a tissue ex vivo. Various embodiments of the method further include culturing the cells in a medium, for example the medium is selected from: growth, chondrogenic, muscle, bone, and adipose. In various embodiments of the method, culturing the cells involves forming a three-dimensional micromass.

An embodiment of the method provides contacting the cells with at least one selected from the group of: a coactivator, a transcription repressor, a transcription enhancer, and a growth factor. Alternatively, the method includes administering at least one of: a growth factor, an anti-inflammatory agent, a vasopressor, a collagenase inhibitor, a collagenase, a steroid, a matrix metalloproteinase inhibitor, an ascorbate, an angiotensin, a calreticulin, a tetracycline, a fibronectin, a collagen, a thrombospondin, a transforming growth factor, a keratinocyte growth factor, a fibroblast growth factor, an insulin-like growth factor (IGF), an IGF binding protein, an epidermal growth factor, a platelet derived growth factor, a neu differentiation factor, a hepatocyte growth factor, a vascular endothelial growth factor, a heparin-binding epidermal growth factor, a thrombospondins, a von Willebrand Factor-C, a heparin, a heparin sulfate, and a hyaluronic acid. In related embodiments of the method, contacting the cells optionally further includes administering at least one agent selected from: an anti-tumor, an antiviral, an antibacterial, an anti-mycobacterial, an anti-fungal, an anti-proliferative and an anti-apoptotic.

An embodiment of the method provides engineering the modulator by constructing an siRNA that specifically targets a nucleic acid having a sequence encoding the transcription factor or encoding the agent that binds to the transcription factor. Alternatively, engineering the modulator includes constructing an antibody or portion thereof that specifically targets the cells or a surface antigen on the cells. For example, engineering the antibody includes synthesizing a monoclonal antibody or a polyclonal antibody.

In various embodiments of the method, measuring the amount of the at least one phenotype of chondrocyte, muscle, or bone (i.e., chondrocyte, muscle, or bone phenotype) includes measuring an amount of at least one from the group of: a myosin for example myosin heavy chain or myosin light chain; an actin; an actin/myosin complex; a collagen; a hyaluronan; an aggrecan; a paired box protein for example paired box (Pax) 3 or Pax 7; an alkaline phosphatase; an osteocalcin; and a procollagen type 1 N-terminal propeptide.

The method further includes after measuring the amount of the at least one chondrocyte, muscle, or bone phenotype, measuring an amount of remediation of a disease or condition selected from heterotopic ossification; edema; formation of a mass of tissue comprising cartilaginous material or bone material; joint or muscle stiffness; joint or muscle pain; arthritis; bone fracture such as in the tibia, fibula, or femur. In various embodiments of the method, observing involves imaging a site of the subject, for example using magnetic resonance imaging, X-ray imaging, and fluorescence imaging. An embodiment of the method includes manually palpitating a site in the subject at which the cells are located, for example manipulating a tissue such as a joint or a bone.

The method in an embodiment further includes observing the localization of the modulator, for example by visualizing a detectable marker bound or fused to the modulator, for example the detectable marker is selected from the group consisting of: detectable, fluorescent, colorimetric, enzymatic, radioactive, and the like. For example, the detectable marker is a green fluorescent protein or a cyanine 3 fluorescent dye.

An aspect of the invention provides a method for identifying a modulator of trans-differentiation of muscle satellite cells including: contacting a first sample of cells with a potential modulator; inducing trans-differentiation of the first sample of cells; and, measuring an amount of at least one of a chondrocyte phenotype, a muscle phenotype, and a bone phenotype, in comparison to at least one phenotype of a second sample of cells induced to trans-differentiate and not so contacted with the modulator and otherwise identical, such that an increase or a decrease in the phenotype in the first sample of cells compared to the second sample of cells identifies the modulator.

In various embodiments, after measuring the amount of the at least one of the chondrocyte phenotype, the muscle phenotype, and the bone phenotype, the method further comprises comparing the phenotype of the first sample of cells to a third sample of cells contacted with a control and then induced to trans-differentiate, such that the control includes an expression vector for example the expression vector optionally further includes a nucleic acid that encodes a transcription factor or a reporter agent for example a fluorescent agent, a colorimetric agent, an enzymatic agent, or a radioactive agent.

An embodiment of the method further includes inducing trans-differentiation of the first sample of cells, contacting the first sample of cells with a BMP, for example BMP-4, or a transforming growth factor such as transforming growth factor beta.

In various embodiments of the method, measuring the amount of the at least one of the chondrocyte phenotype, the muscle phenotype, and the bone phenotype further comprises measuring at least one molecular such as a glycoprotein; a glycosaminoglycan, a sugar, and a nucleic acid. In various embodiments, measuring includes determining an amount or a relative amount of the glycoprotein and/or a glycosaminoglycan, for example determining the amount and/or the relative amount of a collagen, a hyaluronan, an aggrecan, a brevican, or a neurocan.

In various embodiments of the method, measuring the phenotype further includes measuring at least one protein of: myosin for example myosin heavy chain or myosin light chain; an actin; an actin/myosin complex; and a paired box protein for example Pax1, Pax2, Pax3, Pax4, Pax5, Pax6, Pax7, or Pax8.

In various embodiments of the method, measuring the amount of the phenotype further includes measuring at least one protein selected from: alkaline phosphatase, osteocalcin, and procollagen type 1 N-terminal propeptide.

In various embodiments of the method, measuring the amount of the at least one of the phenotype further includes visualizing the 1st, 2nd, and 3^{rd} samples of cells by at least one technique selected from: immunostaining, radiography, microscopy, and photography.

In an embodiment of the invention, the control comprises an NKX protein, a Sox protein, or a portion thereof.

An aspect of the invention provides a kit for modulating trans-differentiation of muscle satellite cells, the kit including: a modulator of trans-differentiation of the muscle satellite cells selected from the group of: a transcription factor, a nucleic acid sequence encoding expression of the transcription factor, and an agent that binds to the transcription factor, such that the transcription factor is a homeodomain class transcription factor such as Nkx3.2 or a TATA binding protein class transcription factor such as Sox9, and includes at least one nucleotide binding-domain; the kit further including a container and instructions for use.

In an embodiment of the kit, the transcription factor includes an NKX protein or a portion thereof. In various embodiments of the kit, the NKX protein optionally further includes at least one sequence selected from the group of: SEQ ID NO: 41, SEQ ID NO: 42, SEQ ID NO: 59, SEQ ID NO: 60, SEQ ID NO: 69, SEQ ID NO: 70, and substantially identical. For example, the nucleic acid sequence encoding expression of the NKX protein, for example a Nkx3.2 protein, is selected from the group of: SEQ ID NO: 41, SEQ ID NO: 59, and SEQ ID NO: 69. In various embodiments of the kit, the NKX protein comprises an amino acid sequence selected from the group of: SEQ ID NO: 42, SEQ ID NO: 60, SEQ ID NO: 70, and substantially identical.

In an embodiment of the kit, the transcription factor includes a Sox protein or a portion thereof. In various embodiments of the kit, the Sox protein optionally further includes at least one sequence selected from the group of: SEQ ID NO: 43, SEQ ID NO: 44, SEQ ID NO: 61, SEQ ID NO: 62, SEQ ID NO: 71, SEQ ID NO: 72, and substantially identical. In various embodiments of the kit, the nucleic acid sequence encoding expression of the Sox protein, for example a Sox9 protein, is selected from the group of: SEQ ID NO: 43, SEQ ID NO: 61, and SEQ ID NO: 71. In various embodiments of the kit, the Sox protein comprises an amino acid sequence selected from the group of: SEQ ID NO: 44, SEQ ID NO: 62, SEQ ID NO: 72, and substantially identical.

The kit includes in the pharmaceutical any of the embodiments described herein of the modulator. For example, the modulator includes a NKX protein, a Sox protein, or a portion thereof.

An embodiment of the agent that binds to the transcription factor in the kit includes a repressor that binds to a Nkx3.2 protein, a Sox9 protein, or a portion thereof. An embodiment of the kit includes the agent that negatively modulates expression of the transcription factor.

Embodiments of the kit have the instructions for use that include instructions for a composition or a method for modulating trans-differentiation of muscle satellite cells of a subject, or include instructions for a composition or a method for stimulating formation of cartilage and/or bone in the subject. The kit in various embodiments optionally further includes an applicator for contacting or administering the pharmaceutical composition to cells or to a tissue of a subject. An embodiment of the kit includes at least one applicator selected of: a bottle, a sprayer, a fluid dropper, a solution dropper, an inhaler, a gauze, a strip, a brush, a spatula, a tweezer, a pipette, and a syringe.

An embodiment of the kit further includes a substrate or a material for attaching to the modulator prior to contacting the modulator to the cells or the tissue. For example the modulator is applied to the substrate or the material for at least a minute, an hour, a day, or a week, for subsequent contact to the cells or tissue.

An aspect of the invention provides a method for stimulating formation of cartilage and/or bone in a subject including: contacting cells or a tissue of the subject with a modulator of trans-differentiation of muscle satellite cells, such that the modulator is selected from the group of: a transcription factor, a nucleic acid encoding expression of the transcription factor, and an agent that binds to the transcription factor, the transcription factor being selected from a homeodomain class transcription factor and a TATA binding protein class transcription factor and includes at least one nucleotide binding-domain; and, the method optionally further comprising measuring initiation of cartilage formation or bone formation in the cells or the tissue.

An embodiment of the method further includes prior to contacting the cells or the tissue, engineering the modulator for example by constructing a gene encoding an amino acid sequence comprising the modulator. For example, engineering the modulator includes synthesizing at least one of: an NKX protein or a portion thereof; a recombinant NKX protein gene encoding a deletion or modification of a terminal end of the amino acid sequence or protein domain; a fusion protein comprising a Nkx3.2 protein or portion thereof and a VP16 transcriptional domain; a Sox protein or portion thereof; a mutated Sox gene including a deletion or modification of a terminal end of the amino acid sequence or protein domain.

An embodiment of the method includes engineering the modulator by constructing a nucleic acid vector carrying the gene encoding the transcription factor, or constructing a viral vector carrying a gene encoding the transcription factor.

An embodiment of the method of engineering the transcription factor further includes expressing a fusion protein in the cells for example the fusion protein comprises at least one of a Nkx3.2 protein or portion thereof, and a VP16 transcriptional domain.

An embodiment of the method of contacting the cells further includes contacting the cells or the tissue in situ or in vivo. For example, contacting the cells includes injecting the modulator into a joint, a muscle, or a bone. Alternatively, contacting the cells includes administering the modulator to an adjacent tissue or adjacent area such that the modulator diffuses within the subject.

In an embodiment of the method, contacting the cells or the tissue is ex vivo. For example, the method includes contacting in a cell culture or a medium. In an embodiment of the method, contacting includes incubating the modulator in a cell culture containing or including the cells or the tissue.

In an embodiment of the method, contacting with the modulator involves contacting stem cells such as embryonic stem cells or adult stem cells; satellite cells such as muscle satellite cells; or progenitor cells. In various embodiments of the method, the cells are at least one selected from the group of mammals and non-mammals such as: human, murine, bovine, porcine, ovine, simian, and avian. In various embodiments of the method, the subject is a mammal for example a human or a mouse.

The method optionally further includes contacting the cells with at least one selected from the group of: a coactivator, a transcription repressor, a transcription enhancer, and a growth factor.

In various embodiments the method of engineering the modulator further includes constructing an siRNA that specifically targets the nucleic acid encoding the transcription factor, or constructing a nucleic acid encoding the agent that binds to the transcription factor.

The method in various embodiments further includes measuring or observing an amount of forming the cartilage or the bone in the cells or the tissue of subject. For example, measuring or observing includes monitoring tissue formation (e.g., cartilage, bone or muscle) for at least a day, a week, a month, or a year.

The method in various embodiments involves after contacting observing increased or decreased expression of a marker. In various embodiments, observing expression of the marker includes detecting at least one of: a myosin; a myosin heavy chain; a myosin light chain; an actin; an actin/myosin complex; a collagen; a hyaluronan; an aggrecan; a paired box protein, Pax3, Pax 7; an alkaline phosphatase; an osteocalcin; and a procollagen type 1 N-terminal propeptide.

In various embodiments of the method, contacting the subject with the modulator includes at least one route of administration selected from the group of: topical, ocular, nasal, bucal, oral, rectal, parenteral, intracisternal, intravaginal, and intraperitoneal.

In various embodiments of the method, contacting includes, for example injecting is at least one selected from the group of: intramuscular, intra-cartilaginous, intra-bone, subcutaneous, and intravenous.

In an embodiment of the method, engineering the modulator includes expressing in the cells a gene encoding an NKX protein or a portion thereof, for example an NKX family of homeodomain-containing transcription factors such as NK3 homeobox 2 (Nkx3.2). In an embodiment of the method, engineering further includes mutating a gene encoding an Nkx3.2 protein including deleting or modifying a portion of the gene encoding a carboxy-terminal (C-terminal) domain of the protein. For example, the method involves deleting or modifying the last 20 amino acids, 30 amino acids, 40 amino acids, 50 amino acids, or 60 amino acids of the C-terminal domain.

The transcription factor in an embodiment of the method includes an NKX protein or a portion thereof. In various embodiments, the Nkx protein optionally further includes at least one sequence selected from the group of: SEQ ID NO: 41, SEQ ID NO: 42, SEQ ID NO: 59, SEQ ID NO: 60, SEQ ID NO: 69, SEQ ID NO: 70, and substantially identical. In various embodiments of the method, the nucleic acid sequence encoding expression of the NKS protein, for example a Nkx3.2 protein, is selected from the group of: SEQ ID NO: 41, SEQ ID NO: 59, SEQ ID NO: 69, and substantially identical. In various embodiments of the method, the NKX protein comprises an amino acid sequence selected from the group of: SEQ ID NO: 42, SEQ ID NO: 60, SEQ ID NO: 70, and substantially identical.

The transcription factor in an embodiment of the method includes a Sox protein or a portion thereof. In various embodiments, the Sox protein optionally further includes at least one sequence selected from the group of: SEQ ID NO: 43, SEQ ID NO: 44, SEQ ID NO: 61, SEQ ID NO: 62, SEQ ID NO: 71, SEQ ID NO: 72, and substantially identical. In various embodiments of the method, the nucleic acid sequence encoding expression of the Sox protein, for example a Sox9 protein, is selected from the group of: SEQ ID NO: 43, SEQ ID NO: 61, and SEQ ID NO: 71. In various embodiments of the method, the Sox protein includes an amino acid sequence selected from the group of: SEQ ID NO: 44, SEQ ID NO: 62, SEQ ID NO: 72, and substantially identical.

In embodiments of the method, engineering the modulator includes mutating a gene encoding a Sox protein. In an embodiment of the method, the gene and the Sox protein optionally further include at least one sequence selected from the group of: SEQ ID NO: 43, SEQ ID NO: 44, SEQ ID NO: 61, SEQ ID NO: 62, SEQ ID NO: 71, SEQ ID NO: 72, and substantially identical.

The method in various embodiments optionally further includes observing or measuring remediation of a disease or condition for example heterotopic ossification; edema; formation of a mass of tissue comprising cartilaginous material or bone material; joint stiffness; muscle stiffness; joint pain; cartilage pain; muscle pain; and arthritis.

An aspect of the invention provides a product containing a modulator of trans-differentiation of the muscle satellite cells, such as the modulator is selected from the group of: a transcription factor, a nucleic acid encoding expression of the transcription factor, and an agent that binds to the transcription factor, and the transcription factor is selected from a homeodomain class transcription factor and a TATA binding protein class transcription factor, and includes at least one nucleotide binding-domain. In various embodiments of the product, the modulator is any of the modulators described herein for example in a pharmaceutical composition.

An aspect of the invention provides use of any pharmaceutical composition described herein in the preparation of a medicament for promoting trans-differentiation of cells or tissue.

In various embodiments of the use, the cells or the tissue include at least one selected from the group of: fat, cartilage, muscle, and bone. In various embodiments of the use, the cells or the tissue include stem cells, muscle satellite cells, or progenitor cells.

### BRIEF DESCRIPTION OF THE DRAWINGS

FIG. 1 is a set of photomicrographs and bar graphs of muscle satellite cells isolated from pectoralis muscles of late stage chicken embryos, and then cultured as a three-dimensional (3D) micromass in chondrogenic induction medium containing transforming growth factor beta 3 (TGFβ3) or regular/control medium. Immunostaining and quantitative reverse transcriptase polymerase chain reaction (qRT-PCR) data were performed at the outset (day zero) and in the 3D micromass to determine the amount of muscle satellite cell markers and markers indicative of cell differentiation to mature cartilage. The cultured cells were analyzed for quantities of expression of Pax3, Pax7, myosin heavy chain, myoblast determination protein 1, collagen II, Nkx3.2, Sox9. The muscle satellite cells were stained with Alcian blue for identifying mucopolysaccharides and glycosaminoglycans, and with 4′,6-diamidino-2-phenylindole (DAPI), a compound that forms fluorescent complexes with natural double-stranded DNA. Data show that isolated muscle satellite cell was redirected toward a cartilage phenotype at the expense of the default muscle phenotype.

FIG. 1 panel A is a set of photomicrographs showing Pax 3 and Pax 7 expression in isolated muscle satellite cells at day zero. The photographs in the first row show immunocytochemical staining of cells with antibodies specific to Pax3 (left) and antibodies specific to Pax7 (right). Pax3 and Pax7 are protein markers for muscle satellite cells. The photographs in the second row show DAPI fluorescence of the muscle satellite cells shown in FIG. 1 panel A first row. The photographs in the third row show an overlay of immunocytochemical staining photographs (FIG. 1 panel A first row) and the DAPI staining photographs (FIG. 1 panel A second row). Data show that the isolated chicken muscle satellite cells at day zero were more than 95% positive for Pax3 and Pax7 (FIG. 1 panel A top row, red staining). Thus, the isolated cells were observed to be muscle satellite cells.

FIG. 1 panel B is a bar graph showing relative Pax3 (left) and Pax7 (right) RNA expression levels on the ordinate (relative mRNA level) for chicken embryonic fibroblasts (CEF) and muscle satellite cells (DO Sat) at day zero. Unless otherwise indicated in figures herein, relative expression using qRT-PCR was obtained by normalizing data to expression of control gene/vector carrying glyceraldehyde 3-phosphate dehydrogenase (GADPH). Analysis using qRT-PCR showed that isolated muscle satellite cell markers express Pax3 and Pax7 at day zero, and chicken embryonic fibroblasts cultured in chondrogenic medium did not express muscle markers Pax3 and Pax7. * denotes p<0.05 in statistical analysis.

FIG. 1 panel C is a set of photomicrographs showing expression of Pax 3 (left, first row), Pax7 (middle, first row), and myosin heavy chain (MHC; right, first row) in muscle satellite cell micromass cultured in control medium (Regular Medium; left column) or in chondrogenic induction medium containing TGFβ3 (right column). The data show much greater Pax3, Pax 7, and MHC immunocytochemical staining for muscle satellite cells cultured in control culture medium compared to muscle satellite cells cultured in chondrogenic induction medium (FIG. 1 panel C first row). The photographs in the second row show fluorescence using DAPI of the muscle satellite cells shown in FIG. 1 panel A first row. The immunocytochemistry data show a dramatic downregulation of Pax3, Pax7, and MHC in muscle satellite cells cultured in chondrogenic induction medium compared to muscle satellite cells cultured in control culture medium. Clearly the chondrogenic induction medium produced chondrogenic stimuli that affected the expression pattern of muscle satellite cells by downregulating Pax3, Pax7 and MHC markers corresponding to muscle cell differentiation.

FIG. 1 panel D is a bar graph showing relative mRNA expression of Pax 3 (Pax3, left), Pax7 (Pax7, second from left), myoblast determination protein 1 (myoD, third from the left), and myosin heavy chain (MHC; right) in muscle satellite cell micromass cultured in control medium (Regular Medium; left column) or in chondrogenic culture medium containing TGFβ3 (right column). Analysis using qRT-PCR showed decreased expression of muscle markers Pax3, Pax7, MyoD, and MHC muscle satellite cells cultured in chondrogenic induction medium compared to muscle satellite cells cultured in control culture medium. * denotes p<0.05 in statistical analysis.

FIG. 1 panel E is a set of photomicrographs showing expression of collagen II (Col II, left), and staining using Alcian Blue (right) in muscle satellite cell micromass cultured in control culture medium (Regular Medium; left column) or in chondrogenic induction medium containing TGFβ3 (Chondro medium, right column). Immunocytochemistry analysis of the sectioned cultures showed increased collagen II protein expression in muscle satellite cell micromass cultured in chondrogenic induction medium compared to control medium. Alcian blue staining of the muscle satellite cell micromass cultured in chondrogenic induction medium showed increased glycosaminoglycan levels compared to muscle satellite cell micromass cultured in control medium.

FIG. 1 panel F is a bar graph showing relative mRNA expression of Nkx3.2 (Nkx3.2, left), Sox9 (Sox9, second from left), collagen II (Collagen II, third from the left), and aggrecan (Aggrecan; right) on the ordinate in muscle satellite cell micromass cultured in control medium (Regular Medium; left column) or in chondrogenic culture medium containing TGFβ3 (right column) on the abscissa. The qRT-PCR data show increased expression of cartilage markers Nkx3.2, Sox9, collagen II, and aggrecan in muscle satellite cell cultured in chondrogenic induction medium compared to in muscle satellite cell cultured in control culture medium. * denotes p<0.05 in statistical analysis.

FIG. 2 is a set of bar graphs showing expression levels of collagen II, aggrecan, myoD, myogenin, and MHC in muscle satellite cell 3D micromass contacted with avian-specific retrovirus encoding transcription factor Pax3 or control alkaline phosphatase (AP). Data show that viral delivery of a gene that encodes Pax3 in muscle satellite cells resulted in inhibition of chondrogenesis and maintenance of muscle gene expression. Unless otherwise indicated in figures herein, relative expression using qRT-PCR was obtained by normalizing data to expression of control vector carrying glyceraldehyde 3-phosphate dehydrogenase (GADPH). * denotes p<0.05 in statistical analysis.

FIG. 2 panel A is a bar graph showing relative collagen mRNA expression levels (ordinate) in a muscle satellite cell 3D micromass that was contacted with a vector encoding alkaline phosphatase (AP) or Pax3 (abscissa). Increased aggrecan expression was observed in muscle satellite cells contacted with a vector encoding Pax3 and compared to muscle satellite cells contacted with a vector encoding alkaline phosphatase.

FIG. 2 panel B is a bar graph showing relative aggrecan mRNA expression levels (ordinate) in muscle satellite cells contacted with a vector encoding alkaline phosphatase (AP) or Pax3 (abscissa) compared to control muscle satellite cells. An increased relative aggrecan mRNA level was observed in muscle satellite cells contacted with a vector encoding alkaline phosphatase compared to muscle satellite cells contacted with a vector encoding Pax3.

FIG. 2 panel C is a bar graph showing mRNA expression of myoD (left), myogenin (middle) and MHC (right) on the ordinate in muscle satellite cells contacted with a vector having a nucleic acid sequence that encodes alkaline phosphatase (AP; left bar) or Pax3 (right bar) on the abscissa. Cells contacted with a vector encoding Pax3 induced greater amounts of myoD, myogenin, and MHC mRNA compared to cells contacted with a vector encoding alkaline phosphatase.

FIG. 3 is a set of photomicrographs and bar graphs showing immunochemical analysis and expression levels in muscle satellite cells in culture contacted with a vector encoding Nkx3.2 protein, or Sox9 protein, or a control vector encoding GFP. A sample of the muscle satellite cells was contacted with both a vector encoding Nkx3.2 and a vector encoding Sox9. In figures herein, the vector encoding amino acid encoding Nkx3.2 includes an amino acid sequence encoding a human influenza hemagglutinin (HA; SEQ ID NO: 56) epitope tag. Accordingly the vectors encoding Nkx3.2 are referred to in FIG. 3 as Nkx3.2 and Nkx3.2HA, respectively. In FIG. 3 herein, a vector encoding Sox9 includes an amino acid sequence encoding a V5 (GKPIPNPLLGLDST; SEQ ID NO: 73) epitope tag, which is derived from the V protein of simian virus 5. (SV5). Accordingly the vectors encoding Sox9 are referred to in FIG. 3 as Sox9, and Sox9V5, respectively. Immunostaining was performed using DAPI or an antibody specific for either GFP, HA, V5, Pax3, Pax7 or MHC. Immunostaining and qRT-PCR data showed that Nkx3.2 and Sox9 inhibited muscle-specific gene expression in muscle satellite cells. * denotes p<0.05 in statistical analysis.

FIG. 3 panel A is a set of photomicrographs showing expression in muscle satellite cells in culture contacted with a vector carrying a nucleic acid that encodes: Nkx.3.2 (Nkx3.2HA; second column from left), Sox9 (Sox9V5; third column from the left), or GFP (GFP; left column). A sample of cells was contacted with both a vector encoding Nkx3.2 and a vector encoding Sox9 (Nkx3.2HA+Sox9V5; right column). The photographs in the first row show direct GFP fluorescence (left photograph), or immunostaining specific for HA tag (second photograph from the left, and right photo) or for V5 tag (third photograph from the left). The photographs in the second row show Pax3 antibody immunostaining of these cells. The photographs in the third row show fluorescence of cells using DAPI. The photographs in the fourth row show an overlay of immunostaining in FIG. 3 panel A first row photographs and the corresponding Pax3 staining in FIG. 3 panel A second row photographs. FIG. 3 panel A second row shows that contacting muscle satellite cells with a vector having a nucleic acid that encodes Nkx3.2 and cells contacted both a vector encoding Nkx3.2 and a vector encoding Sox9 resulted in reduced Pax3 staining compared to muscle satellite cells contacted with vectors having a nucleic acid that encodes either Sox9 alone or GFP.

FIG. 3 panel B is a bar graph showing relative Pax3 RNA expression levels (ordinate; relative Pax3 mRNA level) in muscle satellite cells that were contacted with a vector encoding Nkx3.2, Sox9, or both (abscissa) compared to control muscle satellite cells. Control cells were contacted with a control vector encoding GADPH. Pax3 RNA expression was lower in cells contacted with a vector encoding Nkx3.2 or in cells contacted both with a vector encoding Nkx3.2 and a vector encoding Sox9, relative to cells contacted with the control vector encoding neither Nkx3.2 nor Sox9, or with the vector encoding Sox9 alone.

FIG. 3 panel C is a set of photomicrographs showing expression in cultured muscle satellite cells contacted with a vector carrying a nucleic acid that encodes: Nkx.3.2 (Nkx3.2HA; second column from left), Sox9 (Sox9V5; third column from the left), or GFP (GFP; left column). A sample of cells was contacted with both a vector encoding Nkx3.2 and a vector encoding Sox9 (Nkx3.2HA+Sox9V5; right column). The photographs in the first row show direct GFP fluorescence (left photograph), or immunostaining specific for HA tag (second photograph from the left, and right photo) or V5 tag (third photograph from the left). The photographs in the second row show Pax7 antibody immunostaining of these cells. The photographs in the third row show fluorescence of cells using DAPI. The photographs in the fourth row show an overlay of immunostaining in FIG. 3 panel C first row photographs and the corresponding Pax7 staining in FIG. 3 panel C second row photographs. FIG. 3 panel C second row shows cells contacted with a vector having a nucleic acid that encodes Nkx3.2, and cells contacted both a vector encoding Nkx3.2 and a vector encoding Sox9 expressed reduced Pax7 compared to muscle satellite cells contacted with a vector carrying either Sox9 or GFP.

FIG. 3 panel D is a bar graph showing relative Pax7 RNA expression levels (ordinate; relative Pax7 mRNA level) in muscle satellite cells contacted with a vector encoding Nkx3.2, Sox9, or both (abscissa) compared to control muscle satellite cells contacted with a control vector encoding GADPH. Pax7 RNA expression was observed to be lower in cells contacted with a vector encoding either Nkx3.2 or in cells contacted both with a vector encoding Nkx3.2 and a vector encoding Sox9 relative to cells contacted with the control vector, or with the vector encoding Sox9 alone.

FIG. 3 panel E is a set of photomicrographs showing expression in muscle satellite cells in culture contacted with a vector carrying: Nkx.3.2 (Nkx3.2HA; second column from left), Sox9 (Sox9V5; third column from the left), or GFP (GFP; left column). The photographs in the first row show direct GFP fluorescence by these cells (left photograph), or immunostaining specific for HA (second photograph from the left, and right photo) or V5 (third photograph from the left). The photographs in the second row show myosin heavy chain antibody immunostaining, indicating that the cells expressed a protein characteristic of mature muscle fibers. The photographs in the third row show fluorescence of cells using DAPI. The photographs in the fourth row show an overlay of immunostaining in FIG. 3 panel E first row photographs and the corresponding Pax7 staining in FIG. 3 panel E second row photographs. FIG. 3 panel E second row shows that muscle satellite cells contacted with a vector encoding Nkx3.2 and cells contacted with both a vector encoding Nkx3.2 and a vector encoding Sox9 had reduced MHC staining compared to muscle satellite cells contacted with a vector carrying either Sox9 or GFP. Data show that Nkx3.2 or a combination of Nkx3.2 and Sox 9 inhibited MHC expression in muscle satellite cells. Thus, either Nkx3.2 or both Nkx3.2 and Sox9 acted to repress ability of muscle satellite cells to differentiate to mature muscle.

FIG. 3 panel F is a bar graph showing relative MHC RNA expression levels (ordinate; relative MHC mRNA level) in muscle satellite cells that were contacted with a vector encoding Nkx3.2, Sox9, or both (abscissa) compared to control muscle satellite cells contacted with a control vector encoding GADPH. MHC RNA expression was observed to be greater in cells contacted with the control vector, or with the vector encoding Sox9 alone compared to cells contacted with a vector encoding either Nkx3.2 or in cells contacted both with a vector encoding Nkx3.2 and a vector encoding Sox9.

FIG. 4 is a set of photomicrographs and bar graphs showing immunochemical analysis and expression levels in muscle satellite cells in culture that were contacted with a vector encoding: Nkx3.2 or portions thereof, GFP, or alkaline phosphatase. Data show that the C-terminus of Nkx3.2 was required to inhibit differentiation of muscle satellite cells to a muscle cell fate. p<0.05 in statistical analysis.

FIG. 4 panel A is a set of photomicrographs showing muscle satellite cells contacted with a vector carrying a gene encoding either: GFP (GFP; left column); Nkx.3.2 (Nkx3.2 HA; second column from left); Nkx3.2 having a deleted C-terminal domain (Nkx3.2ΔC-HA; third column from left); or Nkx3.2 including a deleted C-terminal domain and a VP16 transcriptional activation domain substituted for the C-terminal domain (Nkx3.2ΔC-VP16; right column). The photographs in the first row show direct GFP fluorescence (left photograph), or immunostaining specific for human influenza hemagglutinin (HA; second and third photographs from the left) or VP16 (right photograph). The photographs in the second row show Pax3 antibody immunostaining of these cells. The photographs in the third row show DAPI immunostaining of cells. The photographs in the fourth row are an overlay of the photographs showing staining in FIG. 4 panel A first row and the Pax3 staining in FIG. 4 panel A second row.

FIG. 4 panel B is a bar graph showing relative Pax3 RNA expression levels in muscle satellite cells (ordinate) that were contacted with a vector encoding a protein (abscissa) compared to control muscle satellite cells contacted with a control vector encoding GADPH. The vectors encoded: Nkx3.2 (Nkx3.2 HA; second column from left); Nkx3.2 having a deleted C-terminal domain (Nkx3.2ΔC-HA; third column from left); a fusion protein of Nkx3.2 including a deleted C-terminal domain replaced and a substituted VP 16 transcriptional activation domain for the C-terminal domain (Nkx3.2ΔC-VP16; right column); or alkaline phosphatase (AP; left column). Data show that contacting cells with a vector encoding Nkx3.2 resulted in significantly reduced relative Pax3 RNA levels compared to a vector encoding either Nkx3.2 having a deleted C-terminal domain, or AP. Muscle satellite cells contacted with a vector encoding Nkx3.2 having a deleted C-terminal domain and a VP16 transcriptional activation domain showed increased relative Pax3 RNA levels compared to cells contacted with a vector encoding alkaline phosphatase.

FIG. 4 panel C is a set of photomicrographs showing muscle satellite cells contacted with a vector carrying a nucleic acid that encodes either: GFP (GFP; left column); Nkx.3.2 (Nkx3.2 HA; second column from left); Nkx3.2 having a deleted C-terminal domain (Nkx3.2ΔC-HA; third column from left); or Nkx3.2 including a deleted C-terminal domain and a VP16 transcriptional activation domain substituted for the C-terminal domain (Nkx3.2ΔC-VP16; right column). The photographs in the first row show direct GFP fluorescence (left photograph), or immunostaining specific for HA (second and third photographs from the left) or VP16 (right photograph). The photographs in the second row show Pax7 antibody immunostaining of these cells. The photographs in the third row show the DAPI immunostaining of cells. The photographs in the fourth row are an overlay of the photographs showing staining in FIG. 4 panel C first row and the Pax7 staining in FIG. 4 panel C second row.

FIG. 4 panel D is a bar graph showing relative Pax7 RNA expression levels on the ordinate in muscle satellite cells that were contacted with a vector encoding a protein (abscissa) compared to control muscle satellite cells contacted with a control vector encoding GADPH. The vector encoded either: Nkx3.2 (Nkx3.2 HA; second column from left); Nkx3.2 having a deleted C-terminal domain (Nkx3.2ΔC-HA; third column from left); or a fusion protein of Nkx3.2 including a deleted C-terminal domain replaced and a substituted VP16 transcriptional activation domain for the C-terminal domain (Nkx3.2ΔC-VP16; right column). Cells were contacted with a control vector encoding alkaline phosphatase (AP; left column). It was observed that a vector encoding Nkx3.2 and a vector encoding Nkx3.2 having a deleted C-terminal domain and a VP16 transcriptional activation domain resulted in a reduced relative Pax7 RNA levels compared to a vector encoding either Nkx3.2 having a deleted C-terminal domain, or vector encoding alkaline phosphatase.

FIG. 4 panel E is a set of photomicrographs showing muscle satellite cells contacted with a vector carrying a nucleic acid that encodes either: GFP (GFP; left column); Nkx.3.2 (Nkx3.2 HA; second column from left); Nkx3.2 having a deleted C-terminal domain (Nkx3.2ΔC-HA; third column from left); or Nkx3.2 including a deleted C-terminal domain and a VP16 transcriptional activation domain substituted for the C-terminal domain (Nkx3.2ΔC-VP16; right column). Nkx3.2 having a deleted C-terminal domain and a VP16 transcriptional activation domain substituted for the C-terminal domain is a reverse mutant of Nkx3.2. The photographs in the first row show direct GFP fluorescence (left photograph), or immunostaining specific for HA (second and third photographs from the left) or VP16 (right photograph). The photographs in the second row show MHC antibody immunostaining of these cells. The photographs in the third row show the DAPI immunostaining of cells. The photographs in the fourth row are an overlay of the photographs in FIG. 4 panel E first row and the MHC staining in FIG. 4 panel F second row. It was observed that MHC staining in muscle satellite cells contacted with a vector having a nucleic acid that encodes Nkx3.2 was greatly reduced compared to staining in muscle satellite cells contacted with a vector having a nucleic acid that encodes either Nkx3.2 having a deleted C-terminal domain, and Nkx3.2 including a deleted C-terminal domain and a VP16 transcriptional activation domain, or GFP. Data show that expression of Nkx3.2 protein inhibited MHC expression in muscle satellite cells, and that removing the C-terminal domain from Nkx3.2 reduced or eliminated the inhibitory effect. A fusion gene encoding Nkx3.2 with a deleted C-terminal domain and a VP16 transcriptional activation domain was observed to have enhanced MHC expression. Thus, the Nkx3.2 reverse mutant produced the opposite effect than that observed for Nkx3.2 alone.

FIG. 4 panel F is a bar graph showing relative MHC RNA expression levels in muscle satellite cells (ordinate) that were contacted with a vector encoding a protein (abscissa) compared to control muscle satellite cells not contacted with a vector. The vector encoded either: Nkx3.2 (Nkx3.2 HA; second column from left); Nkx3.2 having a deleted C-terminal domain (Nkx3.2ΔC-HA; third column from left); a fusion protein of Nkx3.2 including a deleted C-terminal domain replaced and a substituted VP16 transcriptional activation domain for the C-terminal domain (Nkx3.2ΔC-VP16; right column); or alkaline phosphatase (AP; left column). Control cells were contacted with a vector carrying GADPH. Data show that contacting cells with a vector encoding Nkx3.2 resulted in reduced relative MHC RNA levels compared to contacting with a vector encoding either Nkx3.2 having a deleted C-terminal domain, or AP. The vector encoding Nkx3.2 having a deleted C-terminal domain and a VP16 transcriptional activation domain resulted in increased relative MHC RNA levels in contacted cells compared to control cells.

FIG. 5 is drawing, a bar graph and a set of photomicrographs showing that Nkx3.2 and Sox9 inhibited mouse Pax3 promoter activity.

FIG. 5 panel A is a drawing of a vector expressing a fusion of genes encoding thymidine kinase (TK) and lucerifase under control of a mouse Pax3 promoter (murine Pax3 promoter).

FIG. 5 panel B is a set of photomicrographs of muscle satellite cells contacted with a vector carrying a nucleic acid that encodes either: GFP (GFP; left column); Sox9 (Sox9, second column from the left); Nkx.3.2 (Nkx3.2; third column from left); Nkx3.2 having a deleted C-terminal domain (Nkx3.2ΔC; fourth column from left); or Nkx3.2 including a deleted C-terminal domain and a VP16 transcriptional activation domain substituted for the C-terminal domain (Nkx3.2ΔC-VP16; right column). The photographs in the first row show direct virus staining of the muscle satellite cells. The cells were visualized using immunofluorescence staining using a primary antibody specific to HA or V5 and then a secondary antibody. Cells contacted with a vector carrying GFP were not stained. The photographs in the second row show MHC antibody immunostaining. The photographs in the second row show the DAPI immunostaining. The photographs in the third row are an overlay of the photographs in FIG. 5 panel B first row and the DAPI staining in FIG. 5 panel B second row. Data show from immunocytochemistry analysis showed that the vector delivery efficiencies to the muscle satellite cells was substantially equivalent.

FIG. 5 panel C is a bar graph showing relative lucerifase amounts (relative luciferase units, RLU; ordinate) for muscle satellite cells contacted with a vector (abscissa) carrying a either: GFP (left column); Sox9 (second column from the left); Nkx.3.2 (third column from left); Nkx3.2 having a deleted C-terminal domain (Nkx3.2ΔC; fourth column from left); or Nkx3.2 including a deleted C-terminal domain and a VP16 transcriptional activation domain substituted for the C-terminal domain (Nkx3.2ΔC-VP16; right column), and then transfected with the Pax3 luciferase construct shown in FIG. 5 panel A. A control luciferase vector (pGL3) was used for normalization. * denotes p<0.05 in statistical analysis. Data show muscle satellite cells contacted with Sox9, Nkx3.2 and Nkx3.2 having a deleted C-terminal domain was reduced in RLU and in lucerifase expression compared to control GFP cells. Increased RLU was detected in muscle satellite cells contacted with Nkx3.2 having a deleted C-terminal domain and a VP16 transcriptional activation domain substituted for the C-terminal domain.

FIG. 6 is a set of photomicrographs and bar graphs showing expression levels in muscle satellite cells in culture contacted with a vector encoding transcription factor Nkx3.2, Sox9, or control GFP. A sample of muscle satellite cells were contacted also with both the vector encoding Nkx3.2 and the vector encoding Sox9. Immunostaining and qRT-PCR data both show that Nkx3.2 and Sox9 induced cartilage markers collagen II and aggrecan. Most importantly, contacting muscle satellite cells with both Nkx3.2 and Sox9 resulted in synergistic cartilage formation. * denotes p<0.05 in statistical analysis.

FIG. 6 panel A is a set of photomicrographs showing expression in muscle satellite cells in culture contacted with a vector carrying: Nkx.3.2 (Nkx3.2HA; second column from left), Sox9 (Sox9V5; third column from the left), or GFP (left column). A sample of cells were contacted with both the vector encoding Nkx3.2 and the vector encoding Sox9 (Nkx3.2HA+Sox9V5; right column). The photographs in the first row show direct GFP fluorescence (left), or immunostaining specific for HA (second from the left, and right photo) or V5 (third from the left). The photographs in the second row show collagen II antibody immunostaining. The photographs in the third row show fluorescence of DAPI. The photographs in the fourth row show an overlay of immunostaining in FIG. 6 panel A first row photographs and the corresponding collagen II staining in FIG. 6 panel A second row photographs.

FIG. 6 panel B is a bar graph showing relative collagen mRNA expression levels (ordinate) in muscle satellite cells that contacted with a vector encoding Nkx3.2 and/or a vector encoding Sox9 (abscissa) compared to control muscle satellite cells not contacted with a vector. Control cells were contacted with a control vector encoding GADPH. A synergistic increase in relative collagen II mRNA levels was observed for muscle satellite cells contacted with a vector encoding Nkx3.2 or a vector encoding Sox9.

FIG. 6 panel C is a bar graph showing relative aggrecan mRNA expression levels (ordinate) in muscle satellite cells contacted with a vector encoding Nkx3.2 and/or a vector Sox9 (abscissa) compared to control muscle satellite cells contacted with a vector encoding GADPH. Cells were contacted with a control empty vector (encoding neither Nkx3.2 nor Sox9). Data show that a synergistic increase in relative aggrecan mRNA level was observed in muscle satellite cells contacted with both a vector encoding Nkx3.2 and a vector encoding Sox9.

FIG. 7 is a set of photomicrographs and bar graphs showing expression levels in muscle satellite cells in culture contacted with each of a vector encoding transcription factor Nkx3.2, Sox9, or portions thereof, or control GFP or AP. Both immunostaining and qRT-PCR data show that Nkx3.2 and Sox9 acted synergistically to promote cartilage formation in muscle satellite cells. Nkx3.2 and Sox9 induced cartilage markers collagen II and aggrecan. Contacting muscle satellite cells with both a vector carrying a reverse function Nkx3.2 mutant, Nkx3.2ΔC-VP16, and Sox9 prevented Sox9 from inducing collagen II and aggrecan mRNA levels and protein expression. These data show that Nkx3.2 was required for Sox9 to activate a cartilage program and to inhibit the muscle program in muscle satellite cells.

FIG. 7 panel A is a bar graph showing Nkx3.2 mRNA expression (ordinate) in muscle satellite cells contacted with a vector (abscissa) carrying Sox9 (right) or control GFP (left). Data show that contacting cells with the vector carrying Sox9 increased Nkx3.2 expression compared to cells contacted with the control vector carrying GFP.

FIG. 7 panel B is a bar graph showing Sox9 mRNA expression (ordinate) in muscle satellite cells contacted with a vector (abscissa) carrying Nkx3.2 (right) or GFP (left). Data show that cells contacted with the vector encoding Nkx3.2 induced greater amounts of Sox9 mRNA compared to cells contacted with the vector encoding GFP.

FIG. 7 panel C is a set of photomicrographs showing muscle satellite cells contacted with combinations of vectors: GFP and AP (left column); AP and Sox9 (AP+Sox9V5, second column from the left); Nkx.3.2 and Sox9 (Nkx3.2HA+Sox9V5; third column from left); or Nkx3.2 having a deleted C-terminal domain and a VP16 transcriptional activation domain substituted for the C-terminal domain and Sox9 (Nkx3.2ΔC-VP16 Sox9V5; right column). The photographs in the first row show GFP fluorescence (FIG. 7 panel C first row, left photograph) and immunostaining specific for the V5 epitopic tag (V5; FIG. 7 panel C first row, second and third photographs from left and right photograph). The photographs in the second row show collagen II immunostaining. The photographs in the third row show the DAPI immunostaining. The photographs in the fourth row are an overlay of the photographs in FIG. 7 panel C first row and the collagen II staining in FIG. 7 panel C second row. The immunocytochemistry data showed increased collagen II expression in muscle satellite cells contacted with a vector encoding Sox9 and a vector encoding any of AP, Nkx3.2, and Nkx3.2 including a deleted C-terminal domain and a VP16 transcriptional activation domain substituted for the C-terminal domain.

FIG. 7 panel D is a bar graph showing collagen II mRNA expression (Col II mRNA expression; ordinate) in muscle satellite cells contacted with mixtures of vectors (abscissa): Sox9 vector alone (second column from left); both Sox9 and Nkx3.2 having a deleted C-terminal domain vectors (third column from left); both Sox9 and Nkx3.2 having a deleted C-terminal domain and a VP16 transcriptional activation domain substituted for the C-terminal domain vectors (right column); or GFP vector alone (left column). Greater collagen II mRNA was observed in cells contacted with vectors encoding Sox9 and Nkx3.2 having a deleted C-terminal domain compared to cells contacted with Sox9 alone, or vectors encoding Sox9 and Nkx3.2 including a deleted C-terminal domain and a VP16 transcriptional activation domain, or control vector encoding GFP.

FIG. 7 panel E is a bar graph showing aggrecan mRNA expression (Agg mRNA expression; ordinate) in muscle satellite cells contacted with mixtures of vectors (abscissa): Sox9 alone (second column from left); both Sox9 and Nkx3.2 having a deleted C-terminal domain (third column from left); both Sox9 and Nkx3.2 having a deleted C-terminal domain and a VP16 transcriptional activation domain substituted for the C-terminal domain (right column); or control GFP (left column). Data show that greater aggrecan mRNA levels were observed in cells contacted with both vectors encoding each of Sox9 and Nkx3.2 having a deleted C-terminal domain compared to Sox9 alone, or both Sox9 and Nkx3.2 having a deleted C-terminal domain and a VP16 transcriptional activation domain, or control GFP.

FIG. 7 panel F is a bar graph showing Pax3 mRNA expression (ordinate) in muscle satellite cells contacted with a mixture of vectors carrying: Sox9 alone (second column from left); both Sox9 and Nkx3.2 having a deleted C-terminal domain (third column from left); both Sox9 and Nkx3.2 having a deleted C-terminal domain and a VP16 transcriptional activation domain substituted for the C-terminal domain (right column); or GFP (left column). Pax3 mRNA enhanced expression was observed in cells contacted with either Sox9 alone, and even greater enhancement was observed in cells contacted with both Sox9 and Nkx3.2 having a deleted C-terminal domain and a VP16 transcriptional activation domain substituted for the C-terminal domain. Little or no change in Pax3 mRNA amount was detected in muscle satellite cells contacted with Sox9 and Nkx3.2 including a deleted C-terminal domain and a VP16 transcriptional activation domain, compared to GFP.

FIG. 7 panel G is a bar graph showing myosin heavy chain mRNA expression (MHC mRNA expression; ordinate) in muscle satellite cells contacted with a mixture of vectors (absciss): Sox9 alone (second column from left); both Sox9 and Nkx3.2 having a deleted C-terminal domain (third column from left); both Sox9 and Nkx3.2 having a deleted C-terminal domain and a VP16 transcriptional activation domain substituted for the C-terminal domain (right column); or GFP (left column). Increased amounts of myosin heavy chain mRNA were detected for muscle satellite cells contacted with both Sox9 and Nkx3.2 having a deleted C-terminal domain and a VP 16 transcriptional activation domain, compared to GFP. Data show that myosin heavy chain mRNA amounts were reduced in cells contacted with either Sox9 alone, and Sox9 and a vector encoding Nkx3.2 having a deleted C-terminal domain compared to cells contacted with GFP.

FIG. 7 panel H is a set of photomicrographs showing muscle satellite cells contacted with a vector carrying a nucleic acid that encode either control alkaline phosphatase (AP; left column); Nkx.3.2 (Nkx3.2 HA; second column and third column from left); or a fusion protein of Nkx3.2 having a deleted C-terminal domain and a VP16 transcriptional activation domain substituted for the C-terminal domain (Nkx3.2ΔC-VP16; first column and second column from right). The first row shows direct alkaline phosphatase fluorescence of the cells. The second row shows collagen H antibody immunostaining of these cells. The third row shows labeled DNA fluorescence of these cells. The fourth row shows an overlay of GFP fluorescence (first row) and the MHC antibody staining (second row). Cells contacted with Nkx3.2 were observed to express much greater collagen II compared to cells contacted with a vector having a nucleic acid that encodes either a fusion protein of Nkx3.2 including a deleted C-terminal domain and a VP16 transcriptional activation domain, or control alkaline phosphatase. Thus, vectors encoding Nkx3.2 induced collagen II expression and positively modulated trans-differentiation of muscle satellite cells to cartilage, and vectors encoding Nkx3.2 including a deleted C-terminal domain inhibited collagen II expression and negatively modulated trans-differentiation of muscle satellite cells to cartilage.

FIG. 8 is drawing, photomicrographs and a set of bar graphs and showing that Nkx3.2 and Sox9 are induced in the muscle progenitor cells that contribute to cartilage formation in an in vivo model of fracture healing.

FIG. 8 panel A is a drawing showing generation of the transgenic mice in which MyoD+ lineage cells are labeled with heat-resistant alkaline phosphatase (hPLAP). MyoD-cre Z/AP reporter mice were bred by crossing MyoD-cre (white mouse) and Z/AP (shaded mouse). The murine lines were combined using a Cre-Lox recombination system. Symbols: human placental alkaline phosphatase (hPLAP); chicken beta-actin promoter (CH β-actin promoter); locus of X-over P1 (loxP); myoblast determination protein 1 (myoD); and Cre recombinase (Cre).

FIG. 8 panel B is a set of photomicrographs of hematoxylin and eosin (H&E; top row) staining and immunohistochemistry analysis for heat-resistant alkaline phosphatase (HI-AP; bottom row) for a murine subject one week post-fracture. Each imaged was viewed at different magnifications: four-fold (4×, left column) and ten-fold (10×, right column). The H&E analysis showed the fracture callus site one week post-fracture. Muscle progenitor cells were identified by assaying for heat-resistant alkaline phosphatase (FIG. 8 panel B bottom row; arrow). Symbols: B, bone; C, callus; and M, muscle.

FIG. 8 panel C is a set of photomicrographs showing collagen and Sox9 expression and DAPI staining in the fracture callus of subjects one week after fracture. The photographs in first row show collagen II (left) and Sox9 (right) immunocytochemical staining (FIG. 8 panel C first row). The photographs in the second row show the DAPI immunostaining (FIG. 8 panel C second row). The photographs in the third row are an overlay of the immunocytochemical staining photographs in FIG. 8 panel C first row and the DAPI staining in FIG. 8 panel C second row. Data show increased collagen II expression cells of the fracture callus.

FIG. 8 panel D shows qRT-PCR analysis of muscle progenitor cells in the fracture callus and in neighboring muscle. Laser capture micro-dissection (LCM) procedure was used to obtain targeted analysis of relative mRNA levels (ordinate) of muscle progenitor cells in the fracture callus (left column) and in the neighboring muscle (right column). Expression was determined using qRT-PCR analysis for the following genes on the ordinate: Nkx3.2, Sox9, collagen II (Col II), Pax3, Pax7 and myosin heavy chain (MHC). For qRT-PCR, 18S RNA was used for normalization. * denotes p<0.05 in statistical analysis. Data show increased expression of Nkx3.2, Sox9, collagen H in the fracture callus compared to the muscle. Cells in the muscle had greater expression levels of proteins Pax3, Pax7 and MHC compared to the cells in the fracture callus.

FIG. 9 depicts an alignment of amino acid sequences Nkx3.2 proteins of: chicken Nkx3.2 protein (SEQ ID NO: 42; 333 amino acids); mouse (SEQ ID NO: 60; 333 amino acids); and human (SEQ ID NO: 70; 333 amino acids). Comparison of the proteins shows substantial homology (identity and similarity) between each of chicken, mouse, and human proteins.

FIG. 10 depicts an alignment of amino acid sequences of Sox9 proteins of: chicken (SEQ ID NO: 44; 494 amino acids); mouse (SEQ ID NO: 62; 507 amino acids); and human (SEQ ID NO: 72; 509 amino acids). Comparison of the proteins shows substantial homology (identity and similarity) between each of chicken, mouse, and human proteins.

FIG. 11 is a set of CLUSTAL W alignments of the amino acid sequences of Nkx3.2 proteins of: chicken (SEQ ID NO: 42); mouse (SEQ ID NO: 60); and human (SEQ ID NO: 70). Comparison of the amino acid sequences shows substantial homology (identity and similarity) between each of chicken, mouse, and human proteins. The star symbol (*) indicates identical residues; dot (.) and colon (:) indicate similar amino acids, and very similar amino acids. It was observed that Nkx3.2 sequences are strongly conserved among the mammalian and warm-blooded animals. The figure also shows that the majority of non-identical residues are conservative changes, for example, leucine and isoleucine, leucine and valine, and alanine and threonine.

FIG. 11 panel A shows alignment of amino acid sequences of chicken (SEQ ID NO: 42) and mouse Nkx3.2 proteins (SEQ ID NO: 60).

FIG. 11 panel B shows alignment of amino acid sequences of chicken (SEQ ID NO: 42) and human Nkx3.2 proteins (SEQ ID NO: 70).

FIG. 11 panel C shows alignment of amino acid sequences of mouse (SEQ ID NO: 60) and human Nkx3.2 proteins (SEQ ID NO: 70).

FIG. 12 is a set of CLUSTAL W alignments of the amino acid sequences of Sox9 proteins of: chicken (SEQ ID NO: 44); mouse (SEQ ID NO: 62); and human (SEQ ID NO: 72). Comparison of the amino acid sequences shows substantial homology (identity and similarity) between each of chicken, mouse, and human proteins. The star symbol (*) indicates identical residues; dot (.) and colon (:) indicate similar amino acids, and very similar amino acids. It was observed that Sox9 sequences are strongly conserved among the mammalian and warm-blooded animals. The figure also shows that the majority of non-identical residues are conservative changes, for example, leucine and isoleucine, leucine and valine, and alanine and threonine.

FIG. 12 panel A shows alignment of amino acid sequences of chicken (SEQ ID NO: 44) and mouse Sox9 proteins (SEQ ID NO: 62).

FIG. 12 panel B shows alignment of amino acid sequences of chicken (SEQ ID NO: 44) and human Sox9 proteins (SEQ ID NO: 72).

FIG. 12 panel C shows alignment of amino acid sequences of mouse (SEQ ID NO: 62) and human Sox9 proteins (SEQ ID NO: 72).

### DETAILED DESCRIPTION

The complexity of muscle satellite cells trans-differentiation occurs in a complex environment location of surrounding cells and tissue having multiple cell types and cell signals. Muscle satellite cells are localized along the surface of muscle fibers under the basal lamina, which is a major component of the extracellular matrix (ECM) and contains proteins including laminin, collagen, and proteoglycans. Mechanical, electrical and chemical signals from the host fiber are directed to the cells through this tissue. Muscle satellite cells are in close contact with the vascular system, as 68% of human satellite cells and 82% of mouse satellite cells are localized within five micrometers of neighboring capillaries and vascular endothelial cells. Clearly, muscle satellite cells constantly receive stimulus from the surrounding environment including host muscle fibers, the circulation system, and ECM. See Kuang, S. et al., 2008 Cell Stem Cell 2: 22-31. The complexity of the environment has made it difficult to precisely modulate trans-differentiation of muscle satellite cells.

As used herein, a “trans-differentiation” refers to a physiological process that occurs during cellular development, and involves alteration of cell fate, e.g., trans-differentiation of any type of somatic cell into any other type of cell, the type referring to tissue specificity.

Muscle satellite cells are the tissue specific stem cells in the adult skeletal muscle that lie beneath the basement membrane of the muscle fiber and are usually mitotically quiescent [1]. The satellite cells re-enter the cell cycle and give rise to differentiated myocytes upon injury or when challenged with a variety of mechanical or biochemical stimuli. The differentiated myocytes form new muscle fibers or fuse with existing fibers, and contribute to muscle growth and repair [1]. Satellite cells from the trunk and the limb are derived from an embryonic population of progenitor cells in the somites, transient mesodermal structures that develop on either side of the neural tube [1]. The embryonic progenitor cells express transcription factors Pax3 and Pax7, which are important for muscle differentiation and survival [2] and for specifying the muscle satellite cell population responsible for postnatal growth [1,3]. Satellite cells that are activated rapidly initiate myoblast determination protein 1 expression, and activation of myogenin and terminally differentiated structural muscle genes such as myosin heavy chain (MHC) [1,3]. Although not expressed in the quiescent satellite cells in the adult, myoblast determination protein 1 is transiently expressed in the satellite cell progenitors in the embryo. Thus, satellite cells may be derived from committed embryonic precursors of myogenic lineage [4,5].

Satellite cells were initially considered to be unipotent stem cells with the ability to generate a unique specialized phenotype, the skeletal muscle cells. However, satellite cells have subsequently been shown to have the ability to adopt alternative cell fates/types, such as the adipogenic fate, as Pax7(+) satellite cells isolated from single myofibers adopted adipogenic fate, in addition to muscle fate in vitro [6,7]; and the osteogenic fate, as muscle satellite cells have been shown to be induced by BMPs to differentiate into osteoblasts in culture [7,8,9,10].

Satellite cells have the ability to form cartilage cells. In vivo, Pax7(+) satellite cells contributed to cartilage growth in salamanders during limb regeneration after amputation [7,11]. Lineage-labeled satellite cells express cartilage marker collagen II in a mouse model of fracture healing [12] [13]. Satellite cells accumulate in callus tissue of the fracture site, exhibit typical morphology of chondrocytes and participate in cartilage formation, which is an essential step in fracture healing [14,15]. Introduction of a physical barrier (i.e., a cell impermeable membrane) between the muscle and fractured bone results in impaired fracture healing [16]. However, improved fracture healing was observed for isolated muscle infected with BMP2, which serves as a superior agent/bridge for fracture repair [17]. L6 myoblasts and C2C12 myoblasts treated with demineralized bone matrix or bone morphogenic protein BMP2 differentiate in vitro into chondrocytes [18,19,20,21].

Without being limited by any particular theory or mechanism of action, it is here envisioned that different modulators have the ability to induce muscle satellite cells or myoblasts to undergo chondrogenic differentiation, and that these modulators play an important role in cartilage formation and regeneration during fracture healing. The molecular mechanisms by which muscle satellite cells adopt a cartilage fate still remain unknown. TGF-beta/BMP signaling was shown to be important in this process, however very little is known about how downstream intracellular factors regulate cell fate transition in muscle progenitor cells.

Molecular events that lead to adoption of cartilage cell fate in muscle satellite cells are shown in examples herein. Two transcription factors Nkx3.2 and Sox9 were shown in examples herein to act downstream of TGF-beta/BMP signaling to regulate the transition from myogenic fate to a chondrogenic fate. Nkx3.2 and Sox9 promoted chondrogenesis in satellite cells, specifically, Nkx3.2 strongly inhibited adoption of muscle cell fate and Sox9 only weakly inhibited myogenesis in satellite cells. A reverse function mutant of Nkx3.2 was observed to block activity of Sox9, indicating that Nkx3.2 was required for Sox9 to promote cartilage formation in satellite cells. Furthermore, data in examples herein showed that muscle-determining factor Pax3 strongly inhibited chondrogenesis. A mouse fracture healing model was used to explore in vivo significance of these transcription factors. The fracture healing model resulted from constructing a genetically modified reporter mouse having muscle progenitor cells that were lineage-traced. It was observed that Nkx3.2 and Sox9 were strongly induced in these progenitor cells, and Pax3 expression was strongly repressed in the descendents of the muscle progenitor cells that contributed to cartilage formation. Thus, data herein show that Nkx3.2, Sox9 and Pax3 acted individually and in combination to modulate chondrogenic differentiation of muscle satellite cells, and that these transcription factors play an important role in the healing process in vivo.

Without being limited by any particular theory or mechanism of action, it is here envisioned that transcription factors Nkx3.2 and Sox9 are involved in calcification processes of tissues such as blood vessels. In calcification processes, signaling events take place that involve these transcription factors which modulate trans-differentiation. Blood vessels for example are a cell type involves in skeletal muscle and smooth muscles. See Collet, G. et al. 2005 Circulation Research 96: 930-938.

Muscle satellite cells make up a stem cell population capable of differentiating into myocytes and contributing to muscle regeneration upon injury. Examples herein analyzed the mechanism by which muscle progenitor cells adopt an alternative cell fate such as the cartilage fate. Muscle satellite cells that normally undergo myogenesis were manipulated using homeodomain class transcription factors and TATA binding protein class transcription factors to express cartilage matrix proteins in vitro in chondrogenic induction medium containing TGFβ3 or BMP2. The myogenic differentiation of the muscle satellite cells was repressed in the muscle satellite cells cultured in chondrogenic induction medium. Furthermore, ectopic expression of myogenic factor Pax3 prevented chondrogenesis in muscle satellite cells. Further transcription factors Nkx3.2 and Sox9 acted downstream of TGFβ3 or BMP2 to promote transition to a chondrogenic cell fate. Nkx3.2 and Sox9 repressed the activity of the Pax3 promoter, and Nkx3.2 strongly acted as a transcriptional repressor. A reverse function mutant of Nkx3.2 blocked the ability of Sox9 to inhibit myogenesis and induce chondrogenesis. Thus, data herein clearly showed that Nkx3.2 was required for Sox9 to promote chondrogenic differentiation in satellite cells. Examples herein further showed constructing an in vivo model of fracture healing including muscle progenitor cells were lineage-traced. Data showed that expression of Nkx3.2 and Sox9 was significantly upregulated in the fracture callus region and that Pax3 was significantly downregulated in the muscle progenitor cells that give rise to chondrocytes during fracture repair. Thus in vitro and in vivo analyses herein showed that Nkx3.2 and Sox9 are modulators of trans-differentiation of muscle satellite cells, and the presence and the balance between these transcription factors is an important indicator of cartilage and muscle formation.

Provided herein are compositions, methods, and kits for modulating trans-differentiation of muscle satellite cells ex vivo and in situ. Examples herein show a modulator of trans-differentiation of the muscle satellite cells. The modulator is a protein, a nucleic acid construct, or a compound that is capable of inducing (positively modulating) or inhibiting (negatively modulating) trans-differentiation of muscle satellite cells to mature muscle, cartilage or bone. The modulator includes: a transcription factor for example Nkx3.2 or Sox9, or a nucleic acid encoding expression of the transcription factor, or an agent that binds to the transcription factor or binds to the nucleic acid.

Ability to modulate muscle satellite cells indicates that these compositions, methods, and kits are capable of preventing and treating diseases and conditions involving aberrant formation of cartilage or bone in soft tissue, and conditions associated with underdeveloped muscle formation in subject. The compositions, methods, and kits herein result in safe and rapid modulation of mammalian muscle satellite cells to treat a wide range of diseases, disorders or conditions including heterotopic ossification.

Patients having heterotopic ossification present with clinical symptoms that generally are one or more abnormal bone formations in tissues such as skin, adjacent to joints, and blood vessels. The factors causing the condition are varied and include: genetic abnormalities, trauma to muscle and soft tissues, injuries to the spinal cord, surgery, and even illness. Heterotopic ossification include conditions myositis ossificans progressive, traumatic myositis ossificans, and neurogenic heterotopic ossification.

Myositis ossificans progressiva, also called fibrodysplasia ossificans progressive results from a rare genetic autosomal dominant disorder that affects I of 2 million persons. Affected individuals are heterozygous, with one normal and one mutated gene, and the condition is characterized by variable expressivity. One half of progeny of affected individuals inherit the disorder, and homozygosity is generally fatal. As the mutated gene determines phenotypic expression, the disorder is characterized as dominant. Kaplan. F. S. et al. March 2008 Best Pract Res Clin Rheumatol 22(1): 191-205.

Traumatic myositis ossificans is characterized by development of a cartilaginous-like mass shortly after a trauma. Within a few days or weeks the mass develops into a solid mass of bone. This type of heterotopic ossification occurs in athletes and is observed in the chest (e.g., pectoralis major), the biceps or the thigh muscles. McCarthy, E. F. et al., 2005 Skeletal Radiol. 34(10): 609-19.

Neurogenic heterotopic ossification is observed in subjects suffering from certain neurological disorders, especially after a spinal cord injury or a head injury. The condition is a frequent complication in spinal cord injury (SCI). It is characterized by the formation of new (ectopic) osseous bone in soft tissue surrounding peripheral joints in patients with the neurologic disorders. Analysis of neurogenic heterotopic ossification in SCI patients indicates that the incidence of the condition ranges from about 10% to about 50%. Recent research has attempted to better diagnose true cases of neurogenic heterotopic ossification associated with no history of muscle trauma, and also to improve diagnosis of the disorder. See Kuijk, A. A. et al., 2002 Spinal Cord 40:313-326. Clinically neurogenic heterotopic ossification is diagnosed as a decreased range of motion in the joints (e.g., jaw, hands, elbows, shoulders, hips and knees) and peri-articular swelling due to interstitial edema of soft tissue.

Heterotopic ossification in addition to the above categories is observed following circumstances including surgery to repair a bone fracture or joint repair. In fact, 60-75% of heterotopic ossification incidence involves trauma to the hip and lower legs, and as many as about 56% of patients having total hip arthroplasty or replacement have a degree of heterotopic ossification. McCarthy, E. F. et al., 2005 Skeletal Radiol. 34(10): 612, 615. Bone formations are observed by X-rays and patients often suffer from piercing pain in the legs and hips, and impaired movement. Clinical analysis also shows that these patients are more likely to require extended periods of hospitalization and rehabilitation after surgery.

Diagnosis of heterotopic ossification includes genetic testing, radiological examination or a three phase bone scan following intravenous injection of radioactive material. Patients suffering from heterotopic ossification are treated with anti-inflammatory agents, pain relievers, and commercially available prescription Didronel®, the disodium salt of 1-hydroxyethylidene diphosphonic acid (Procter & Gamble; Cincinnati, Ohio), which acts to inhibit formation of hydroxyapatite crystals and amorphous precursors by chemical adsorption to calcium phosphate surfaces. Didronal® is used to inhibit heterotopic ossification and has also been used to prevent osteoporosis by promoting bone growth. The product is capable of both producing and to inhibiting bone formation because inhibition of crystal resorption occurs at lower doses than are required to inhibit crystal growth. Thus, the relationship between dosage and bone density is carefully monitored during administration to ensure the desired outcome following treatment.

Radiation therapy is another method used during recent several decades to prevent heterotopic ossification. A patient is administered for example a ionizing radiation 24 hours to 48 hours after surgery and then monitored for symptoms of heterotopic ossification and for negative reactions such as increased bleeding, infection, and impaired wound healing.

There is a need for new more effective and specific methods of treating subjects having diseases and conditions involving trans-differentiation of muscle satellite cells such as heterotopic ossification that involves aberrant bone formation, and muscular dystrophy, an autosomal disease that results in loss of muscle.

As discussed in greater detail in the Examples, pharmaceutical compositions identified by methods herein are useful as modulators of trans-differentiation of stem cells and tissues. The modulators induce or inhibit the trans-differentiation of muscle satellite cells to muscle, chondrocytes or bone, and are useful to treat disease and conditions associated with unwanted bone or cartilage formation in soft tissues including heterotopic ossification. Without being limited by any particular theory or mechanism of action, it is here envisioned that at a cellular level, steps involving initiation and development of trans-differentiation can be described. Initially, muscle stem cells turn off a default muscle program; then the cells turn on an aberrant cartilage program that leads to formation of cartilaginous or bone-like material in the soft tissue.

Transcription factors such as Nkx3.2 and Sox9 shown herein to play an important role in these steps as these proteins include DNA-binding segments that enable attachment to specific genes to regulate the transcription of the specific genes involved in muscle stem cell differentiation.

Nkx3.2 and Sox9 are transcription factors induced by BMP signaling that play roles in cartilage formation and maturation in an early embryo. Mutations in these genes lead to diseases of severe cartilage abnormalities in mammals. Murtaugh, L. C. et al., 2001 Developmental Call 1: 411-422; and Zeng, L. et al., 2002 Genes & Development 16: 1900-2005.

The NK-2 family of homeobox-containing genes (e.g., NKX2-2; NKX2-3; NKX2-4; NKX2-5; NKX2-8; NKX3-1; NKX6-1; NKX6-2 and NKX6-3), has been implicated in human disorders such as congenital anomalies of the heart, cancer, developmental anomalies of the eyes, and in forms of choreoathetosis and hypothyroidism. Hellemans, J. et al., 2009 The American Journal of Human Genetics 85: 916-922. The NKX3-2 (BAPX1) gene in humans is located on chromosome band 4p15.33 and encodes a homeobox-containing protein of 333 amino acids. The homeobox is about 180 base pairs long. It encodes a protein domain (the homeodomain) which when expressed functions to bind to DNA. See Jessell, et al. U.S. Pat. No. 6,955,802 issued Oct. 18, 2005.

Sox genes encode a class of transcription factors that bind specifically to a DNA sequence called a TATA box, which have a core DNA sequence 5′-TATAAA-3′. The TATA box is generally followed by three or more adenine bases and is located 25 base pairs upstream of a transcription site. Sox genes encode proteins that are developmental regulators characterized by the presence of an HMG (high mobility group) DNA-binding domain with more than 50% homology to the sex-determining gene SRY. Sox9 has been associated with heart, hair, neuronal, gonad and pancreas development. Mutations of Sox9 lead to abnormal bone development, perinatal lethality and other abnormalities including tumors of the intestinal epithelium. Bastide, P. et al., 2007 The Journal of Cell Biology vol. 178 (4): 635-648; and Coustry, F. et al., 2010 Nucleic Acids Research vol. 38(18): 6018-6028.

Examples herein show that muscle satellite cells that normally undergo myogenesis can be modulated and/or converted to express cartilage matrix proteins in vitro upon treatment with chondrogenic medium containing TGFβ or BMP2. The muscle satellite cells underwent chondrogenic differentiation during the period of time that myogenesis was repressed.

Furthermore, data herein show that muscle-determining factor Pax3 strongly inhibited chondrogenesis in the muscle satellite cells, and that Nkx3.2 and Sox9 acted downstream of TGFβ3 or BMP to promote transition to a cartilage cell fate. Data show that Nkx3.2 was required for Sox9 to inhibit myogenesis and induce chondrogenesis. In an in vivo model of fracture healing, Nkx3.2 and Sox9 were observed to be significantly and surprisingly upregulated and Pax3 to be significantly downregulated in the muscle progenitor cells that produce chondrocytes. The upregulation of Nkx3.2 and Sox9 and the downregulation of Pax3 correlated with induction of cartilage matrix protein collagen II in lineage-traced muscle progenitor cells. The balance of expression of Pax3, Nkx3.2 and Sox9 played an important role in the cell fate switch of muscle satellite cells from muscle to cartilage. Thus, the balance of the transcriptions factors Pax3, Nkx3.2 and Sox is shown herein to be important in fracture healing in subjects.

Multiple progenitor cell populations are present in the muscle that can be instructed to adopt alternative cell fates. Muscle satellite cells reside underneath the basal lamina of the myocytes [3]. A fibrocyte or adipocyte population (FAP) has been identified in the interstitial spaces of the muscle fibers [48,49]. These progenitor cells do not express muscle satellite cell marker Pax7 or SM/C-2.6, and are positive for expression of Seal, Tie-2 and PDGFR-1a [48,49]. The FAP population differentiates into adipocytes, however this FAP population cannot be induced to differentiate to myogenic or chondrogenic cells. Further, a Sca-1-negative, lin-negative population (i.e. the double-negative (DN) population) in the muscle was found to be capable of differentiating into cartilage and bone, and incapable of differentiating into myocytes [48].

The muscle-derived stem cell population (MDSC) is another progenitor population that resides within the basal lamina unlike muscle satellite cells or the FAP population that are found underneath the basal lamina of the myocytes and in the interstitial spaces of muscle fibers respectively [50, 51]. MDSCs are positive for Sca-1 and negative for Pax7, and have the ability to give rise to muscle, cartilage or bone cells [51]. The muscle satellite cells used in Examples herein are not FAPs and MDSCs, and muscles cells herein express Pax3 and Pax7, and FAP and MDSC cells do not express Pax3 and Pax7.

MyoD(+) progenitors permanently label muscle satellite cells as well as their derivatives in the mature muscle fibers, and muscle progenitor cells do not give rise to non-myogenic adipocytes[4] [52]. However it is not clear whether the muscle satellite cells have the capacity to adopt a chondrogenic or osteogenic fate. Examples herein analyzed the expression of Nkx3.2, Sox9 and Pax3 in the muscle progenitors that contribute to cartilage formation during bone healing during fracture repair. Data herein showed that muscle progenitor cells adopted a cartilage cell fate upon chondrogenic stimulation in vitro, and during open fracture healing in vivo. However, data did not distinguish which specific subpopulations of satellite cells are more likely to undergo chondrogenesis [2]. It is also not clear whether these muscle progenitor cells have undergone de-differentiation/re-differentiation or bone fide transdifferentiation in in vitro cell culture or in vivo fracture healing models. While there was a significant amount of Pax3 and Pax7 protein expression at the beginning of culturing, Pax3 and Pax7 became gradually diminished upon vector delivery of Nkx3.2 and Sox9, concurrently with the induction of cartilage genes, which should be consistent with a transdifferentiation process. Msx1 is correlated with muscle cell dedifferentiation [53,54]. However, msx1 is also highly expressed in chondrocytes and is induced by BMP/TGFβ signaling. Thus, although a significant induction of msx1 expression was observed upon chondrogenic differentiation in the satellite cells, it does not indicate whether the satellite cells have undergone dedifferentiation. Data herein show that muscle progenitor cells that normally would undergo myogenesis, can be redirected to adopt a cartilage cell fate in vitro and in vivo.

Cartilage gene expression in the muscle progenitor cells that contribute to fracture healing was analyzed herein [55]. Without being limited by any particular theory or mechanism of action, it is here envisioned that other cell types located in the vicinity of bone also participate in cartilage and bone formation. Grafting experiments using LacZ-positive donor mice and Lac-Z-negative recipients revealed that cells from the perichondrium, the fibrous covering of the bone, differentiate into chondrocytes and osteocytes during fracture repair [56]. Cells associated with blood vessels, such as pericytes, have also been shown to have the ability to differentiate into chondrocytes [57]. Cells that are positive for Tie-2, an endothelial cell marker, while not yet shown to be recruited to the fracture callus, have been shown to contribute to cartilage and bone formation during heterotopic ossification [58,59]. Thus, different types of cells use different signaling mechanisms when undergoing chondrogenic differentiation because of the diverse cell types that participate in cartilage formation during fracture healing.

TGFβ, BMP, PTH, and Wnt signaling are activated during fracture healing, and downstream molecules such as Smad, prostaglandin, Cox-2 and β-catenin regulate this process [65, 68, 69]. Data herein showed that transcription factors Pax3, Nkx3.2 and Sox9 regulated chondrogenic differentiation of muscle progenitor cells. It is possible that Nkx3.2 and Sox9 also participate in the chondrogenic differentiation of other cell types, such as perichondrial or endothelial cells, and that these different cell types coordinate their signaling events during fracture healing. Without being limited by any particular theory or mechanism of action, it is here envisioned that signaling processes of transcription factors Nkx3.2. and Sox9 in muscle satellite cells result in methods, compositions and kits for accelerating fracture healing in subjects.

Pax3, Nkx3.2 and Sox9 play important roles during development. In embryogenesis, Pax3 is expressed in the dermomyotome of the somite, which gives rise to muscle cell precursors [70]. Pax3 mutant mice exhibited somite truncations with loss of hypaxial dermomyotome, and absence of limb muscle [3]. Data herein elucidate the role of Pax3 in promoting myogenesis in muscle satellite cells [71]. Furthermore, examples herein show that Pax3 has an additional function of inhibiting chondrogenic differentiation of muscle satellite cells. In the double knockout of Pax3 and its paralogue Pax7, significant cell death takes place, leading to the loss of the majority of muscle fibers [3]. Pax3 and Pax7 double mutant cells have been found in the forming rib [3], so that Pax3 and Pax7 may be involved in forming cartilage [25,72]. While Pax3 acts as a transcriptional activator to promote myogenesis [73], it also has a transcriptional repressor domain that is important for the development of melanocytes [76,77,78]. Without being limited by any particular theory or mechanism of action, it is here envisioned that Pax3 inhibited chondrogenesis by acting as a transcriptional repressor or activator in the satellite cells, and that other myogenic factors play inhibitory roles in chondrogenic differentiation.

Sox9 is the master regulator of chondrogenesis, as no cartilage formation takes place in the absence of Sox9 [37]. Sox9 acts as a transcriptional activator in chondrogenic precursor cells by binding to promoters of cartilage-specific matrix genes collagen II and aggrecan [36,44,45]. Examples herein showed that Sox9 strongly induced collagen II and aggrecan expression in the muscle satellite cells, which normally are not chondrogenic precursors. [25]. Data showed also that Sox9 significantly, although weakly, inhibited expression of early muscle lineage marker Pax3 and Pax7, as well as myosin heavy chain. Sox9 is expressed in satellite cells, and has the ability to inhibit α-sarcoglycan expression in the C2C12 myoblast cell line [79] and the myogenin promoter in 10T1/2 cells [80]. Data herein show that Sox9 is much more strongly expressed in chondrocytes, and that ectopic expression of Sox9 leads to chondrogenic differentiation and maintenance of the chondrocyte phenotype.

Examples herein show that Nkx3.2 plays a central role in the chondrogenic differentiation of satellite cells, and Nkx3.2 activity is required for Sox9 to promote chondrogenesis and inhibit myogenesis. Nkx3.2 is expressed in the cartilage precursors in the embryo much like Sox9, and Nkx3.2 promotes cartilage cell fate in the somites [25,26]. Nkx3.2 null mice exhibit reduced cartilage formation including a downregulation of Sox9 expression [39,83,84]. Inactivating mutations of Nkx3.2 in human lead to spondylo-megaepiphyseal-metaphyseal dysplasia (SMMD), a disease that causes abnormalities of the vertebral bodies, limbs and joints [85]. It was observed in examples herein that Nkx3.2 is activated in the muscle satellite cells during chondrogenic differentiation in vitro as well as in the adult fracture healing process in vivo. Thus, Nkx3.2 is involved in a cell fate determination process at a stage later than early embryogenesis. Furthermore, data show that Nkx3.2 acted as a transcriptional repressor to inhibit Pax3 promoter activity.

While there are consensus Nkx3.2 binding sites on the Pax3 promoter, it has not been determined whether Nkx3.2 binds to the Pax3 promoter [28]. Nkx3.2 has also been shown to act as a repressor to inhibit osteogenic determining factor Runx2, however it has not been clearly shown that Nkx3.2 has the ability to inhibit other non-cartilage cell fates [86].

Examples herein elucidated a pivotal role for Nkx3.2 and Sox9 in the induction of chondrogenic genes. It was observed that Sox9, despite its ability to bind to collagen II and aggrecan promoters, was unable to activate those genes or inhibit myogenesis without the repressing activity of Nkx3.2. Additionally, it was observed that Nkx3.2 potentiated the ability of Sox9 to induce aggrecan expression, which may be due to its repression of chondrogenic inhibitor Pax3. Examples herein clearly showed intricate balance of Pax3, Nkx3.2 and Sox9 controlled the determination of cartilage and muscle cell fate in muscle satellite cells, that this balance regulated the process of fracture healing. Without being limited by any particular theory or mechanism of action, it is here envisioned that healing recapitulates development because each of these transcription factors is involved in embryonic cell development. Thus, understanding and utilizing NKX proteins and Sox proteins and the signaling events modulates chondrogenic differentiation to enhance fracture healing.

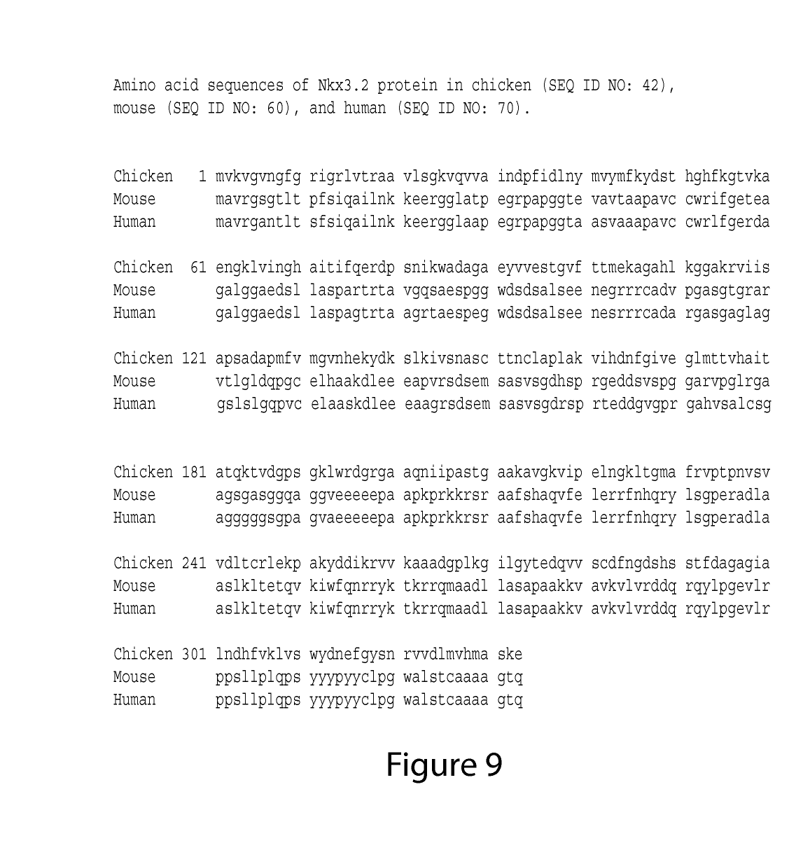

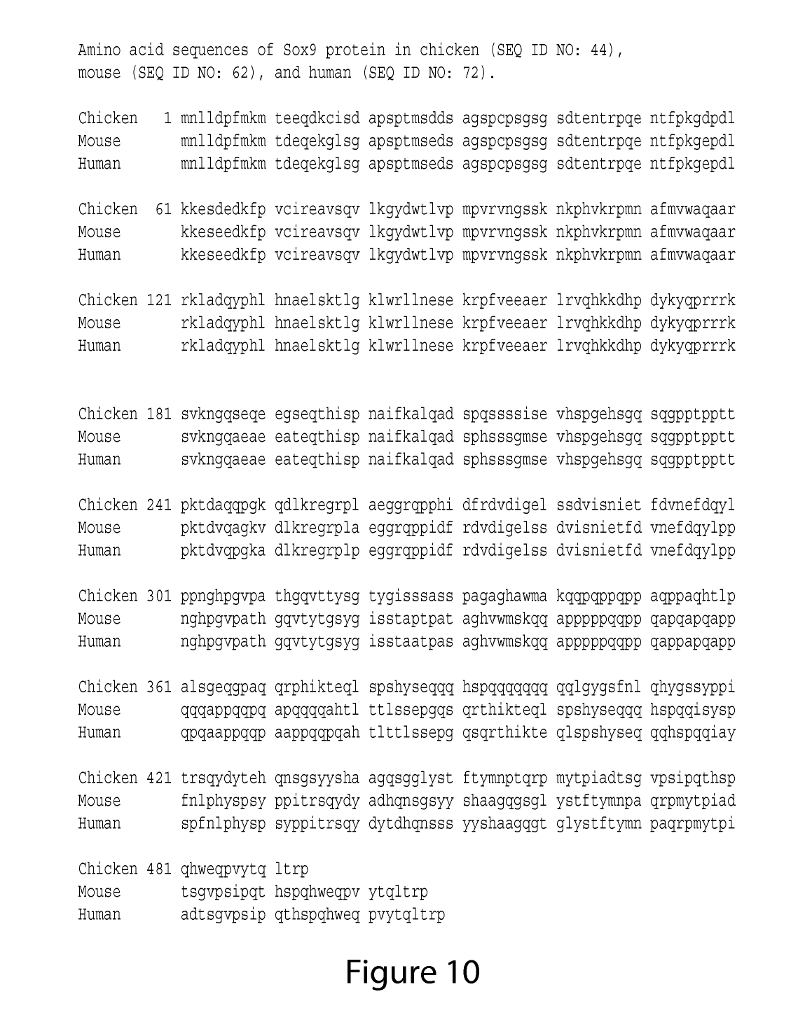

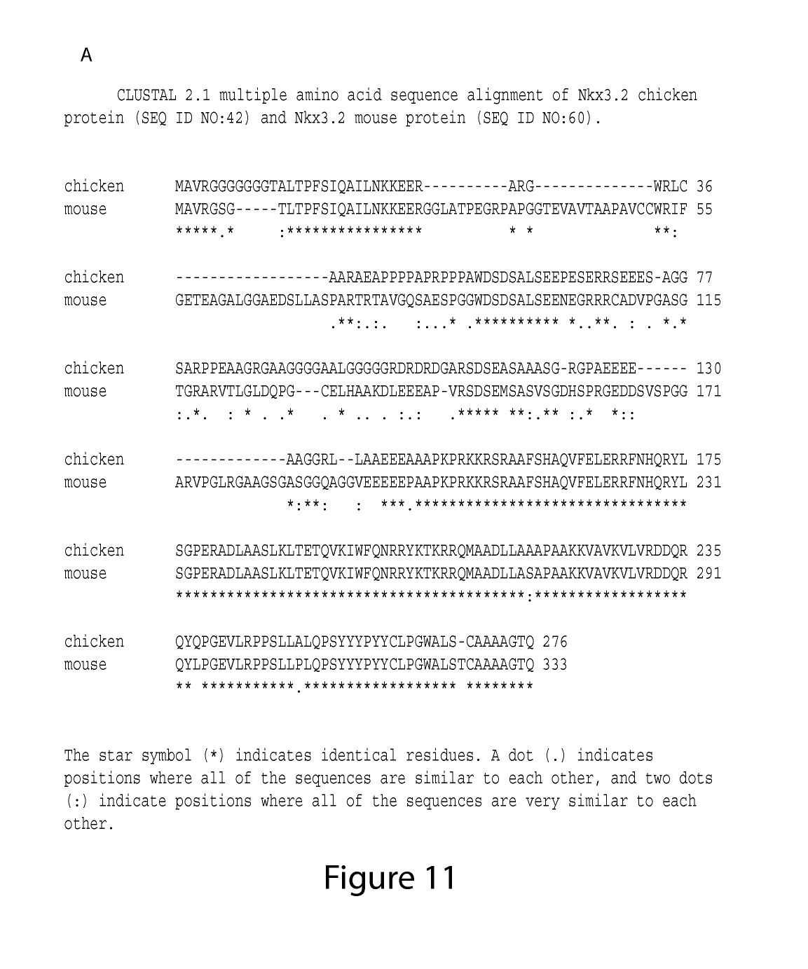

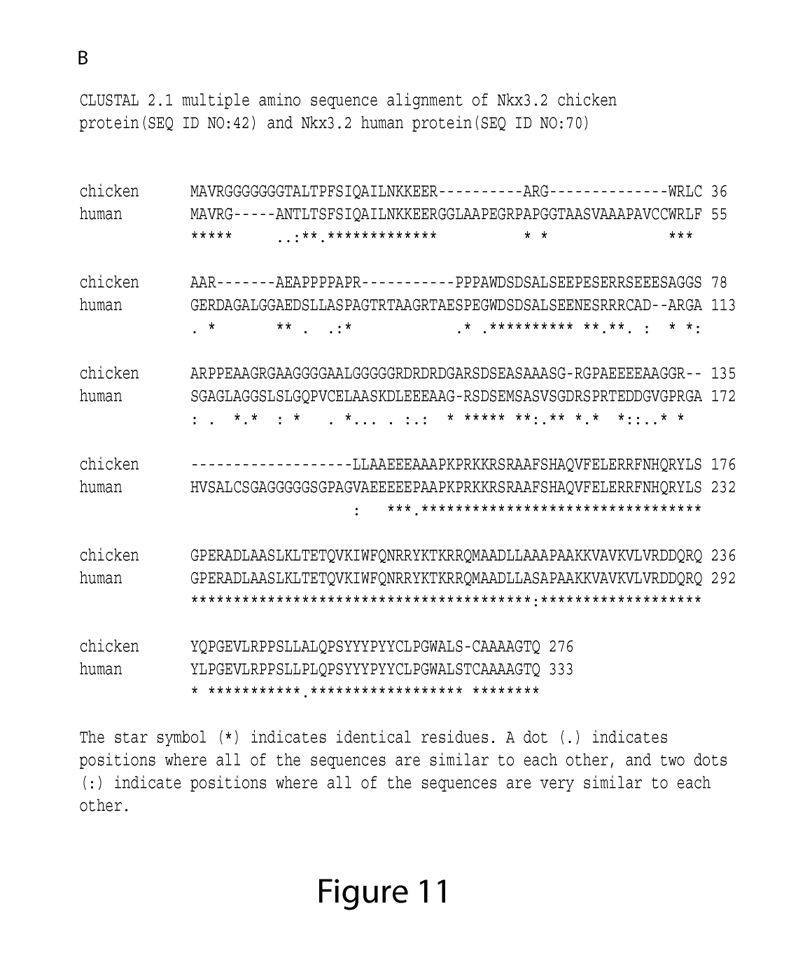

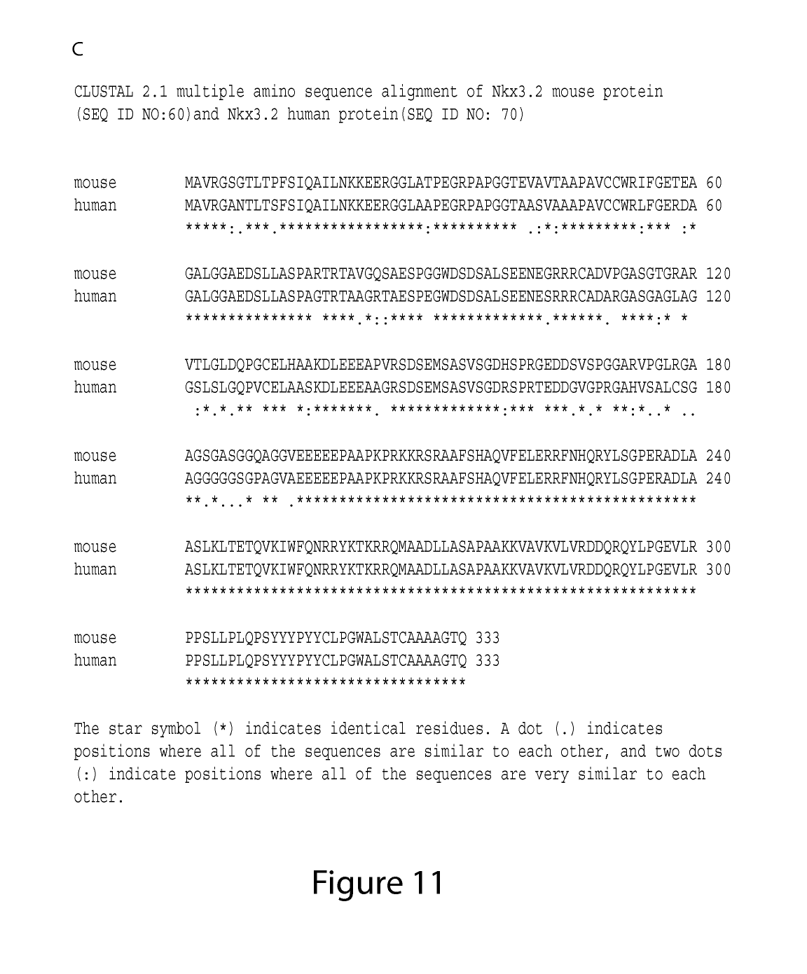

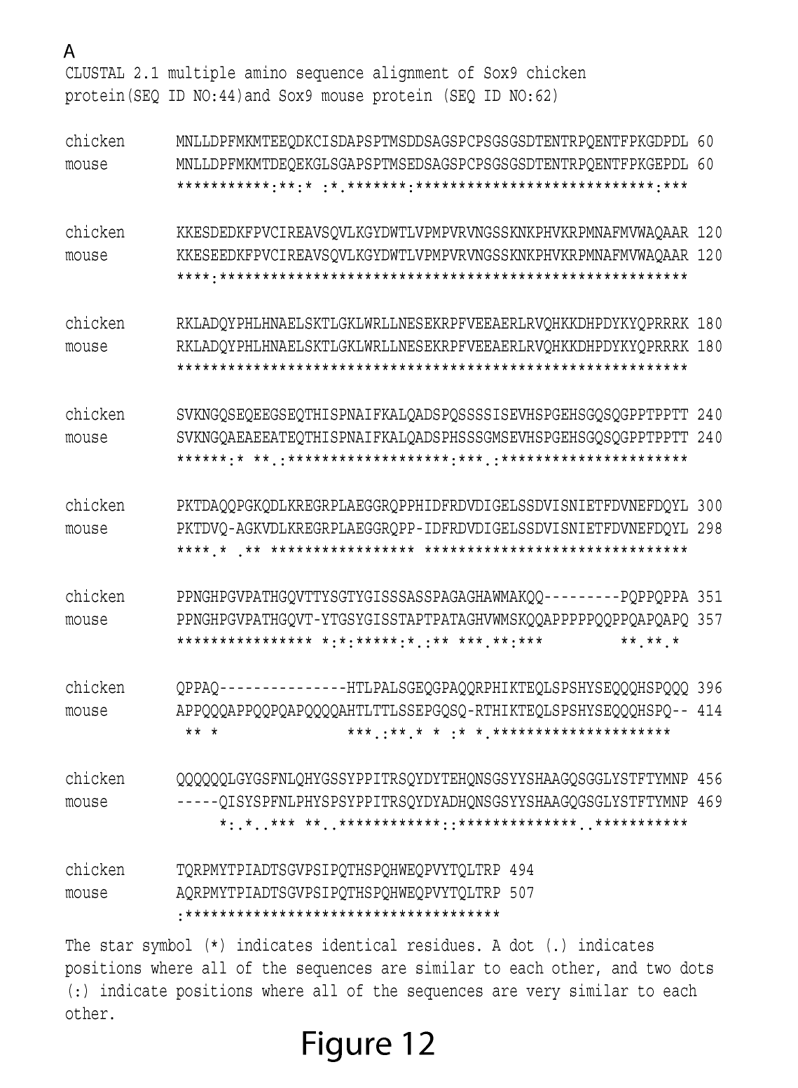

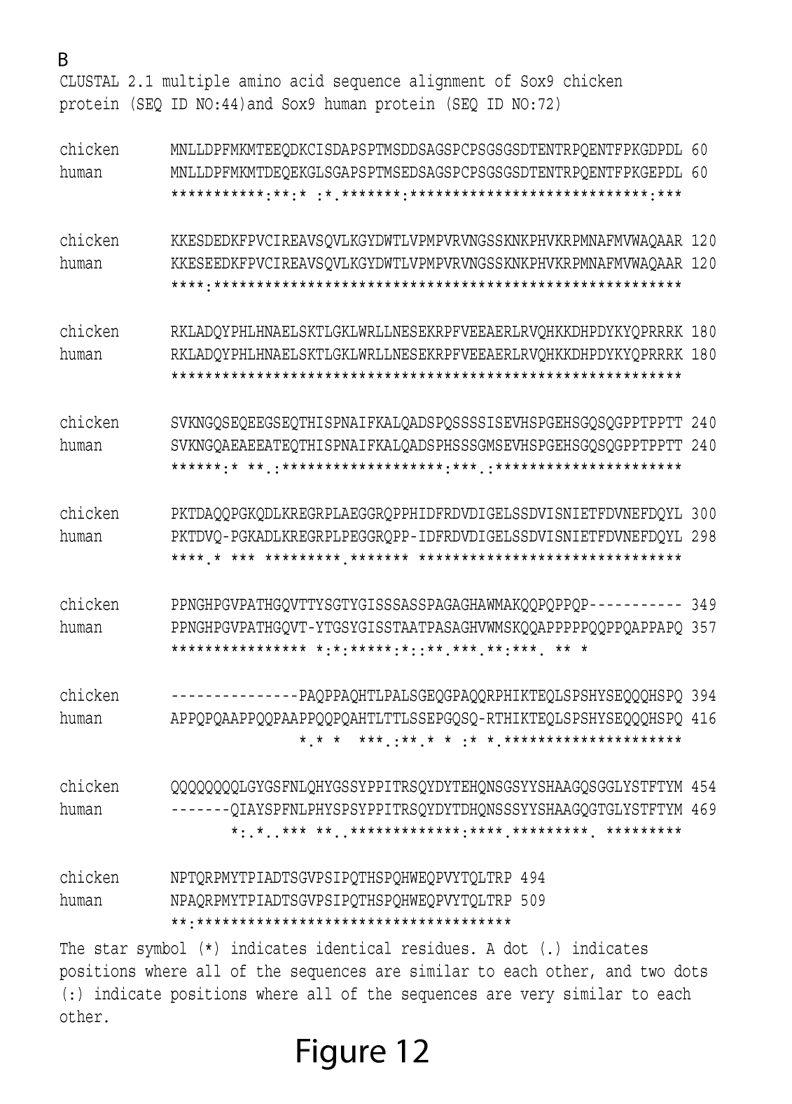

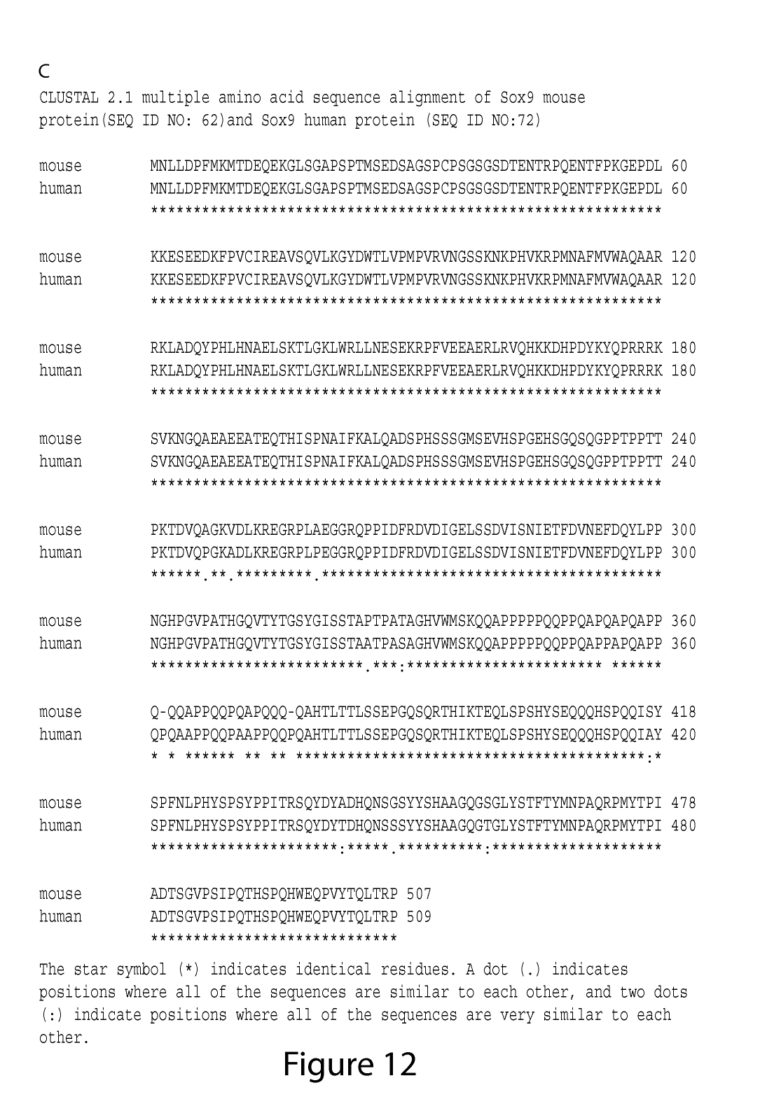

Modulators of trans-differentiation of muscle satellite cells in various embodiments transcriptions of the present invention include the amino acid sequence of transcription factors such as Nkx3.2 protein, Sox9 protein, and portions thereof. Nucleic acid sequences and amino acid sequences of Nkx3.2 protein are shown in SEQ ID NO: 41, SEQ ID NO: 42, SEQ ID NO: 59, SEQ ID NO: 60, SEQ ID NO: 69, SEQ ID NO: 70. Nucleic acid sequences and amino acid sequences of Sox9 protein are shown in SEQ ID NO: 43, SEQ ID NO: 44, SEQ ID NO: 61, SEQ ID NO: 62, SEQ ID NO: 71, SEQ ID NO: 72. Other Nkx3.2 and Sox9 molecules of the present invention include a nucleic acid sequence and an amino acid sequence that is substantially identical to sequence identifications shown herein. In particular, proteins which contain naturally-occurring or engineered induced alterations, such as deletions, additions, substitutions or modifications of certain amino acid residues of Nkx3.2 and/or Sox9 proteins are within the definition of modulators provided herein. It will also be appreciated that as defined herein, Nkx3.2 and Sox9 proteins include regions represented by the amino acid sequences shown herein and wild-type sequences obtained from other mammalian species including but not limited to bovine, canine, feline, caprine, ovine, porcine, murine, and equine species, and also avian species. FIGS. 9 and 11 show the amino acid sequence homology of Nkx.3.2 protein in chicken, mouse, and human species. FIGS. 10 and 12 shows the amino acid sequence homology of Sox9 protein in chicken, mouse, and human species.

Examples herein demonstrate a hierarchy of the roles of homeodomain class transcription factors and TATA binding protein class transcription factors in trans-differentiation of muscle satellite cells to cartilage.

The pathway of muscle stem cells trans-differentiation into cartilage is analyzed. Without being limited by any particular theory or mechanism of action, it is here envisioned that cells that are close in lineage to muscle, such as those of mesenchymal origin, are characterized by differentiation programs that include protection of these cells from aberrant conversion. Muscle satellite cells herein were observed herein not to de-differentiate into cartilage. Data show overlapping expression of muscle marker and cartilage gene expression.

Compositions, methods and kits herein are useful to modulate trans-differentiation of muscle satellite cells using a modulator. As used herein, a “modulator” refers to any molecule, compound, or construct that modulates (increases or decreases) trans-differentiation of muscle satellite cells. The modulator in various embodiments includes a transcription factor, a nucleic acid encoding a molecule (RNA or protein) that modulates expression of the transcription factor, an agent that binds to the transcription factor, and a nucleic acid encoding expression of the agent. For example, the nucleic acid encodes a transcription factor having an amino acid sequence that is substantially identical to the naturally occurring transcription factor. In general a desirable modulator inhibits expression or activity of trans-differentiation.

Analysis of Clustal W alignment of amino acid sequences of Nkx3.2 proteins in FIG. 11 and Sox9 proteins in FIG. 12 are shown in Tables 1-2 respectively.

TABLE 1
Analysis of Clustal W amino acid sequence alignments in FIG. 11 of
Nkx3.2 proteins of chicken (SEQ ID NO: 42), mouse (SEQ ID NO: 60),
and human (SEQ ID NO: 70)

| | number of amino acids from alignment analysis | | |
|---|---|---|---|
| organisms compared | Identical | similar | very similar |
| chicken and mouse | 194 | 26 | 18 |
| chicken and human | 198 | 24 | 18 |
| mouse and human | 283 | 20 | 12 |

TABLE 2
Analysis of Clustal W amino acid sequence alignments in FIG. 12 of
Sox9 proteins of chicken (SEQ ID NO: 44), mouse (SEQ ID NO: 62),
and human (SEQ ID NO: 72)

| | number of amino acids from alignment analysis | | |
|---|---|---|---|
| organisms compared | Identical | similar | very similar |
| chicken and mouse | 433 | 19 | 19 |
| chicken and human | 431 | 19 | 20 |
| mouse and human | 494 | 4 | 4 |

FASTA amino acid alignment analysis shows that amino acid sequences of Nkx3.2 and Sox9 proteins are strongly conserved among human, mouse, and chicken (FIGS. 11-12 and Tables 1-2). Nkx3.2 amino acid sequences for these vertebrates show a very high percentage of identity. Amino acid sequences for chicken and mouse Nkx3.2 proteins are 57.1% identical and 67.3% similar; for chicken and human Nkx3.2 proteins are 58.1% identical and 66.2% similar; and for mouse and human Nkx3.2 proteins are 85.3% identical and 94.0% similar. Thus, Nkx3.2 amino acid sequences share a very high percentage of identity and similarity, and this very high conservation of sequence is true for mammalian and warm-blooded vertebrate species.

The vertebrate species in FIG. 12 and Table 2 share substantial identity in the amino acid sequences of Sox9 protein. The amino acid sequences are highly identical between the two mammalian species and strongly conserved between these mammals and the avian species, viz., conserved among warm-blooded vertebrate species. Amino acid sequences for chicken and mouse Sox9 proteins are 83.6% identical and 89.6% similar. Amino acid sequences for chicken and mouse Sox9 proteins are 83.6% identical and 89.6% similar, and for chicken and human Sox9 proteins are 83.7% identical and 89.8% similar. Mouse and human Sox9 proteins were found to have amino acid sequences that are 97.1% identical and 98.2% similar. The mammalian and warm-blooded Sox9 amino acid sequences share a very high percentage of identity with most of the non-identical residues being conserved amino acid changes, such as glycine at amino acid position 179 in mouse and human Sox9 compared to serine in chicken Sox9 protein.

Without being limited by any theory or particular mode of operation, it is envisioned that that as defined herein, modulators include regions represented by the amino acid sequences of Nkx proteins and Sox proteins taken from other mammalian species and warm blooded species including but not limited to avian, bovine, canine, feline, caprine, ovine, porcine, murine, and equine species, or agents that bind to these sequences.

Modulators of trans-differentiation in examples herein include conservative sequence modifications of naturally occurring transcription factors. As used herein, the term “conservative sequence modifications” refers to amino acid modifications that do not significantly affect the characteristics of the transcription factor and are engineered, for example by substitution of an amino acid with a functionally similar amino acid. Such conservative modifications include amino acid substitutions, additions and deletions. Modification of the amino acid sequence of the modulator is achieved using any known technique in the art e.g., site-directed mutagenesis or PCR based mutagenesis. Such techniques are described in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

In certain embodiments, the amino acid sequence of the modulator is an amino acid sequence that is substantially identical to that of the wild type sequence. The term “substantially identical” is used herein to refer to a first amino acid sequence that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 60% identity, or at least 70%, 75%, 80%, 85%, 90%, 95%, or 99% identity. For example, the modulator has at least 60% identity, at least 65% identity, at least 70% identity, at least 75% identity, at least 80% identity, at least 85% identity, at least 90% identity, at least 95% identity, or at least 99% identity to the amino acid sequence of a wild-type transcription factor Nkx3.2 in a mammal such as a human or a mouse.

Calculations of sequence identity between sequences are performed as follows. To determine the percent identity of two amino acid sequences, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences are accomplished using a mathematical algorithm. Percent identity between two amino acid sequences is determined using an alignment software program using the default parameters. Suitable programs include, for example, CLUSTAL W by Thompson et al., Nuc. Acids Research 22:4673, 1994, BL2SEQ by Tatusova and Madden, FEMS Microbiol. Lett. 174:247, 1999, SAGA by Notredame and Higgins, Nuc. Acids Research 24:1515, 1996, and DIALIGN by Morgenstern et al., Bioinformatics 14:290, 1998.

### Vectors

In various embodiments of the invention herein, a method for modulating trans-differentiation of stem cells, for example muscle satellite cells, is provided, the method including contacting cells or tissue with a pharmaceutical composition including a modulator or a source of modulator expression. For example, the modulator is a recombinantly produced transcription factor protein administered in situ or ex vivo. The term “recombinant” refers to proteins produced by manipulation of genetically modified organisms, for example micro-organisms or eukaryotic cells in culture.

In accordance with the present invention a source of the modulator includes polynucleotide sequences that encode the transcription factor, for example, engineered into recombinant DNA molecules to direct expression of the transcription factor or a portion thereof in appropriate host cells. To express a biologically active transcription factor, a nucleotide sequence encoding the transcription factor, or functional equivalent, is inserted into an appropriate expression vector, i.e., a vector that contains the necessary nucleic acid encoding elements that regulate transcription and translation of the inserted coding sequence, operably linked to the nucleotide sequence encoding the transcription factor amino acid sequence.

Methods that are well known to those skilled in the art are used to construct expression vectors containing a nucleic acid sequence encoding for example a protein or a peptide operably linked to appropriate transcriptional and translational control elements. These methods include in vitro recombinant DNA techniques, synthetic techniques and in vivo recombination or genetic recombination. Techniques are described in Sambrook et al., Molecular Cloning: A Laboratory Manual, Cold Spring Harbor Press, Plainview, N.Y., 1989.

A variety of commercially available expression vector/host systems are useful to contain and express a protein or peptide encoding sequence. These include but are not limited to microorganisms such as bacteria transformed with recombinant bacteriophage, plasmid or cosmid DNA expression vectors; yeast transformed with yeast expression vectors; insect cell systems contacted with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems. See Ausubel et al., Current Protocols in Molecular Biology, John Wiley & Sons, New York, N.Y., 1989.

Virus vectors include, but are not limited to, adenovirus vectors, lentivirus vectors, retrovirus vectors, adeno-associated virus (AAV) vectors, and helper-dependent adenovirus vectors. For example, the vectors deliver a nucleic acid sequence that encodes a transcription factor or agent that binds to a transcription that as shown herein modulates trans-differentation of muscle satellite cells. Adenovirus packaging vectors are commercially available from American Type Tissue Culture Collection (Manassas, Va.). Methods of constructing adenovirus vectors and using adenovirus vectors are shown in Klein et al., Ophthalmology, 114:253-262, 2007 and van Leeuwen et al., Eur. J. Epidemiol., 18:845-854, 2003.

Adenovirus vectors have been used in eukaryotic gene expression (Levrero et al., Gene, 101:195-202, 1991) and vaccine development (Graham et al., Methods in Molecular Biology: Gene Transfer and Expression Protocols 7, (Murray, Ed.), Humana Press, Clifton, N.J., 109-128, 1991). Further, recombinant adenovirus vectors are used for gene therapy (Wu et al., U.S. Pat. No. 7,235,391 issued Jun. 26, 2007).

Recombinant adenovirus vectors are generated, for example, from homologous recombination between a shuttle vector and a provirus vector (Wu et al., U.S. Pat. No. 7,235,391). Helper cell lines for use in these recombinant adenovirus vectors may be derived from human cells such as, 293 human embryonic kidney cells, muscle cells, hematopoietic cells or other human embryonic mesenchymal or epithelial cells. Alternatively, the helper cells may be derived from the cells of other mammalian species that are permissive for human adenovirus, e.g., Vero cells or other monkey embryonic mesenchymal or epithelial cells. Generation and propagation of these replication defective adenovirus vectors using a helper cell line is described in Graham et al, 1997 J. Gen. Virol., 36:59-72, 1977.

Lentiviral vector packaging vectors are commercially available from Invitrogen Corporation (Carlsbad Calif.). An HIV-based packaging system for the production of lentiviral vectors is prepared using constructs in Naldini et al., Science 272: 263-267, 1996; Zufferey et al., Nature Biotechnol., 15: 871-875, 1997; and Dull et al., J. Virol. 72: 8463-8471, 1998.

A number of vector constructs are available to be packaged using a system, based on third-generation lentiviral SIN vector backbone (Dull et al., J. Virol. 72: 8463-8471, 1998). For example the vector construct pRRLsinCMVGFPpre contains a 5′ LTR in which the HIV promoter sequence has been replaced with that of Rous sarcoma virus (RSV), a self-inactivating 3′ LTR containing a deletion in the 113 promoter region, the HIV packaging signal, RRE sequences linked to a marker gene cassette consisting of the Aequora jellyfish green fluorescent protein (GFP) driven by the CMV promoter, and the woodchuck hepatitis virus PRE element, which appears to enhance nuclear export. The GFP marker gene allows quantitation of transfection or transduction efficiency by direct observation of UV fluorescence microscopy or flow cytometry (Kafri et al., Nature Genet., 17: 314-317, 1997 and Sakoda et al., J. Mol. Cell. Cardiol., 31: 2037-2047, 1999).

Manipulation of retroviral nucleic acids to construct a retroviral vector containing a gene that encodes a protein, and methods for packaging in cells are accomplished using techniques known in the art. See Ausubel, et al., 1992, Volume 1, Section III (units 9.10.1-9.14.3); Sambrook, et al., 1989. Molecular Cloning: A Laboratory Manual. Second Edition. Cold Spring Harbor Laboratory Press, Cold Spring Harbor, N.Y.; Miller, et al., Biotechniques. 7:981-990, 1989; Eglitis, et al., Biotechniques. 6:608-614, 1988; U.S. Pat. Nos. 4,650,764, 4,861,719, 4,980,289, 5,122,767, and 5,124,263; and PCT patent publications numbers WO 85/05629, WO 89/07150, WO 90/02797, WO 90/02806, WO 90/13641, WO 92/05266, WO 92/07943, WO 92/14829, and WO 93/14188.

A retroviral vector is constructed and packaged into non-infectious transducing viral particles (virions) using an amphotropic packaging system. Examples of such packaging systems are found in, for example, Miller, et al., Mol. Cell. Biol. 6:2895-2902, 1986; Markowitz, et al., J. Virol. 62:1120-1124, 1988; Cosset, et al., J. Virol. 64:1070-1078, 1990; U.S. Pat. Nos. 4,650,764, 4,861,719, 4,980,289, 5,122,767, and 5,124,263, and PCT patent publications numbers WO 85/05629, WO 89/07150, WO 90/02797, WO 90/02806, WO 90/13641, WO 92/05266, WO 92/07943, WO 92/14829, and WO 93/14188.

Generation of “producer cells” is accomplished by introducing retroviral vectors into the packaging cells. Examples of such retroviral vectors are found in, for example, Korman, et al., Proc. Natl. Acad. Sci. USA. 84:2150-2154, 1987; Morgenstern, et al., Nucleic Acids Res. 18:3587-3596, 1990; U.S. Pat. Nos. 4,405,712, 4,980,289, and 5,112,767; and PCT patent publications numbers WO 85/05629, WO 90/02797, and WO 92/07943.

Herpesvirus packaging vectors are commercially available from Invitrogen Corporation, (Carlsbad, Calif.). Exemplary herpesviruses are an α-herpesvirus, such as Varicella-Zoster virus or pseudorabies virus; a herpes simplex virus such as HSV-1 or HSV-2; or a herpesvirus such as Epstein-Barr virus. A method for preparing empty herpesvirus particles that can be packaged with a desired nucleotide segment is shown in Fraefel et al. (U.S. Pat. No. 5,998,208, issued Dec. 7, 1999).

The herpesvirus DNA vector can be constructed using techniques familiar to the skilled artisan. For example, DNA segments encoding the entire genome of a herpesvirus is divided among a number of vectors capable of carrying large DNA segments, e.g., cosmids (Evans, et al., Gene 79, 9-20, 1989), yeast artificial chromosomes (YACS) (Sambrook, J. et al., Molecular Cloning: A Laboratory Manual, 2nd Edition, Cold Spring Harbor Press, Cold Spring Harbor, N.Y., 1989) or E. coli F element plasmids (O'Conner, et al., Science 244:1307-1313, 1989).

For example, sets of cosmids have been isolated which contain overlapping clones that represent the entire genomes of a variety of herpesviruses including Epstein-Barr virus, Varicella-Zoster virus, pseudorabies virus and HSV-1. See M. van Zijl et al., J. Virol. 62, 2191, 1988; Cohen, et al., Proc. Nat'l Acad. Sci. U.S.A. 90, 7376, 1993; Tomkinson, et al., J. Virol. 67, 7298, 1993; and Cunningham et al., Virology 197, 116, 1993.

AAV is a dependent parvovirus in that it depends on co-infection with another virus (either adenovirus or a member of the herpes virus family) to undergo a productive infection in cultured cells (Muzyczka, Curr Top Microbiol Immunol, 158:97 129, 1992). For example, recombinant AAV (rAAV) virus is made by co-transfecting a plasmid containing the gene of interest, for example, the Nkx3.2 gene. Cells are also contacted or transfected with adenovirus or plasmids carrying the adenovirus genes required for AAV helper function. Recombinant AAV virus stocks made in such fashion include with adenovirus which must be physically separated from the recombinant AAV particles (for example, by cesium chloride density centrifugation).

Adeno-associated virus (AAV) packaging vectors are commercially available from GeneDetect (Auckland, New Zealand). AAV has been shown to have a high frequency of integration and infects nondividing cells, thus making it useful for delivery of genes into mammalian cells in tissue culture (Muzyczka, Curr Top Microbiol Immunol, 158:97 129, 1992). AAV has a broad host range for infectivity (Tratschin et al., Mol. Cell. Biol., 4:2072 2081, 1984; Laughlin et al., J. Virol., 60(2):515 524, 1986; Lebkowski et al., Mol. Cell. Biol., 8(10):3988 3996, 1988; McLaughlin et al., J. Virol., 62(6):1963 1973, 1988).

Methods of constructing and using AAV vectors are described, for example in U.S. Pat. Nos. 5,139,941 and 4,797,368. Use of AAV in gene delivery is further described in LaFace et al., Virology, 162(2):483 486, 1988; Zhou et al., Exp. Hematol, 21:928 933, 1993; Flotte et al., Am. J. Respir. Cell Mol. Biol., 7(3):349 356, 1992; and Walsh et al., J. Clin. Invest, 94:1440 1448, 1994.

Recombinant AAV vectors have been used for in vitro and in vivo transduction of marker genes (Kaplitt et al., Nat Genet., 8(2):148 54, 1994; Lebkowski et al., Mol. Cell. Biol., 8(10):3988 3996, 1988; Samulski et al., EMBO J., 10:3941 3950, 1991; Shelling and Smith, Gene Therapy, 1: 165 169, 1994; Yoder et al., Blood, 82 (Supp.): 1:347 A, 1994; Zhou et al., Exp. Hematol, 21:928 933, 1993; Tratschin et al., Mol. Cell. Biol., 5:3258 3260, 1985; McLaughlin et al., J. Virol., 62(6):1963 1973, 1988) and transduction of genes involved in human diseases (Flotte et al., Am. J. Respir. Cell Mol. Biol., 7(3):349 356, 1992; Ohi et al., Gene, 89(2):279 282, 1990; Walsh et al., J. Clin. Invest, 94:1440 1448, 1994; and Wei et al., Gene Therapy, 1:261 268, 1994).

### Antibodies

The present invention relates also to identifying a potential modulator of trans-differentiation by determining amount of a marker by immunohistochemistry or other analytical technique, using antibodies that are specific for a marker that includes for example a muscle marker, a cartilage marker, or a bone marker. An embodiment of a modulator includes an antibody that binds to a transcription factor. The term “antibody” as referred to herein includes whole antibodies and antigen binding fragments (i.e., “antigen-binding portion”) or single chains of these. A naturally occurring “antibody” is a glycoprotein including at least two heavy (H) chains and two light (L) chains inter-connected by disulfide bonds.

As used herein, an antibody that “specifically binds to a transcription factor” refers to an antibody that binds to a transcription factor with a K_{D} of 5×10⁻⁹ M or less, 2×10⁻⁹ M or less, or 1×10⁻¹⁰ M or less. For example, the antibody is monoclonal or polyclonal. The terms “monoclonal antibody” or “monoclonal antibody composition” as used herein refer to a preparation of antibody molecules of single molecular composition. A monoclonal antibody composition displays a single binding specificity and affinity for a transcription factor or for a particular epitope of a transcription factor. The antibody includes for example an IgM, IgE, IgG such as IgG1 or IgG4.

The terms “polyclonal antibody” or “or polyclonal antibody composition” refer to a large set of antibodies each of which is specific for one of the many differing epitopes found in the immunogen, and each of which is characterized by a specific affinity for that epitope. An epitope is the smallest determinant of antigenicity, which for a protein, comprises a peptide of six to eight residues in length (Berzofsky, J. and I. Berkower, (1993) in Paul, W., Ed., Fundamental Immunology, Raven Press, N.Y., p. 246). Affinities range from low, e.g. 10⁻⁶ M to high, e.g., 10⁻¹¹ M. The polyclonal antibody fraction collected from mammalian serum is isolated by well known techniques, e.g. by chromatography with an affinity matrix that selectively binds immunoglobulin molecules such as protein A, to obtain the IgG fraction. To enhance the purity and specificity of the antibody, the specific antibodies may be further purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. Bound antibodies are eluted from the solid phase by standard techniques, such as by use of buffers of decreasing pH or increasing ionic strength, the eluted fractions are assayed, and those containing the specific antibodies are combined.

Also useful for the methods herein is an antibody that is a recombinant antibody. The term “recombinant human antibody”, as used herein, includes antibodies prepared, expressed, created or isolated by recombinant means. Mammalian host cells for expressing the recombinant antibodies used in the methods herein include Chinese Hamster Ovary (CHO cells) including dhfr-CHO cells, described Urlaub and Chasin, Proc. Natl. Acad. Sci. USA 77:4216-4220, 1980 used with a DH FR selectable marker, e.g., as described in R. J. Kaufman and P. A. Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. To produce antibodies, expression vectors encoding antibody genes are introduced into mammalian host cells, and the host cells are cultured for a period of time sufficient to allow for expression of the antibody in the host cells or secretion of the antibody into the culture medium in which the host cells are grown. Antibodies are recovered from the culture medium using standard protein purification methods.

Standard assays to evaluate the binding ability of the antibodies toward the target of various species are known in the art, including for example, an ELISAs, an western blots and an radio immunoassay (RIA). The binding kinetics (e.g., binding affinity) of the antibodies also can be assessed by standard assays known in the art, such as by Biacore analysis.

General methodologies for antibody production, including criteria to be considered when choosing an animal for the production of antisera, are described in Harlow et al. (Antibodies, Cold. Spring Harbor Laboratory, pp. 93-117, 1988). For example, an animal of suitable size such as a goat, a dog, a sheep, a mouse, or a camel is immunized by administration of an amount of immunogen, such as the intact protein or a portion thereof containing an epitope from a human transcription factor, effective to produce an immune response. An exemplary protocol involves subcutaneous injection with 100 micrograms to 100 milligrams of antigen, depending on the size of the animal, followed three weeks later with an intraperitoneal injection of 100 micrograms to 100 milligrams of immunogen with adjuvant depending on the size of the animal, for example Freund's complete adjuvant. Additional intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the greatest dilution indicating that having a detectable antibody activity. The antibodies are purified, for example, by affinity purification using binding to columns containing human MAC.

Monoclonal antibodies are generated by in vitro immunization of human lymphocytes. Techniques for in vitro immunization of human lymphocytes are described in Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992. These techniques can be used to produce antigen-reactive monoclonal antibodies, including antigen-specific IgG, and IgM monoclonal antibodies. Any antibody or a fragment thereof having affinity and specific for a transcription factor is within the scope of the modulator compositions provided herein.

### RNA Interference

Examples herein include agents that bind to a nucleic acid that encodes proteins such as a transcription factor that modulates trans-differentation of cells for example muscle satellite cells. Methods and compositions for binding to the nucleic acid include utilizing RNA interference (RNAi). RNAi is induced by short (e.g., 30 nucleotides) double stranded RNA (dsRNA) molecules which are present in the cell. These short dsRNA molecules, called short interfering RNA (siRNA) cause the destruction of messenger RNAs (mRNAs) which share sequence homology with the siRNA.

Methods for constructing synthetic siRNA or an antisense expression cassette and inserting it into a recombinantly engineered nucleic acid of a vector are well known in the art and are shown for example in Reich et al. U.S. Pat. No. 7,847,090 issued Dec. 7, 2010; Reich et al. U.S. Pat. No. 7,674,895 issued Mar. 9, 2010; Khvorova et al. U.S. Pat. No. 7,642,349 issued Jan. 5, 2010. For example, the invention herein includes synthetic siRNAs that include a sense RNA strand and an antisense RNA strand, such that the sense RNA strand includes a nucleotide sequence substantially identical to a target nucleic acid sequence in cells. Thus, under the circumstances of cells being contacted with viral vectors encoding the siRNAs, the cells express the siRNAs that then negatively modulate expression of the target nucleic acid sequence.

### Pharmaceutical Compositions

An aspect of the present invention provides pharmaceutical compositions having a modulator that is a transcription factor or a source of expression of the transcription factor, In certain embodiments, these compositions optionally further include one or more additional therapeutic agents, the additional therapeutic agent or agents selected from the group of growth factors, anti-inflammatory agents, vasopressor agents including but not limited to nitric oxide and calcium channel blockers, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid.

In other embodiments, the additional agent is a compound, composition, biological or the like that potentiates, stabilizes or synergizes the ability of the pharmaceutical composition to modulate trans-differentiation of muscle satellite cells. The pharmaceutical composition includes without limitation an anti-tumor, antiviral, antibacterial, anti-mycobacterial, anti-fungal, anti-proliferative or anti-apoptotic agent. See for example, Goodman & Gilman's The Pharmacological Basis of Therapeutics, 9th Ed., Hardman, et al., eds., McGraw-Hill, 1996, the contents of which are herein incorporated by reference herein.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 provides various carriers used in formulating pharmaceutical compositions and known techniques for the preparation thereof. Example of pharmaceutically acceptable carriers are sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; other non-toxic compatible lubricants such as sodium lauryl sulfate and magnesium stearate; coloring agents, releasing agents, coating agents, preservatives and antioxidants according to the judgment of the formulator.

### Therapeutically Effective Dose

Modulation of trans-differentiation by methods provided herein involves contacting cells with a pharmaceutical composition, for example, administering a therapeutically effective amount of a pharmaceutical composition having as an active agent a modulator or a source of expression of a modulator, to a subject in need thereof, in such amounts and for such time as is necessary to achieve the desired result. The modulator is for example a transcription factor or a molecule that binds to the transcription factor.

The compositions, according to the method of the present invention, may be administered using an amount and a route of administration effective for contacting the cells for example muscle satellite cells. Thus, the expression “amount effective for modulating trans-differentiation of muscle satellite cells”, as used herein, refers to a sufficient amount of composition to beneficially prevent, inhibit or otherwise modulate trans-differentiation of the cells.

The exact dosage is chosen by the individual physician in view of the patient to be treated. Dosage and administration are adjusted for sufficient levels of the active agent(s) or to maintain the desired effect. Additional factors that may be taken into account include the severity of the disease state, e.g., intermediate or advanced stage of an ossification syndrome; age, weight and gender of the patient; diet, time and frequency of administration; route of administration; drug combinations; reaction sensitivities; and tolerance/response to therapy. Long acting pharmaceutical compositions are administered hourly, every three to four hours, daily, twice daily, every three to four days, every week, or every two weeks or monthly depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for the patient to be treated. The total daily usage of the compositions of the present invention is decided by the attending physician within the scope of sound medical judgment. For the active agent, the therapeutically effective dose is estimated initially in cell culture assays or in animal models such as mice, rats, rabbits, dogs, or pigs. Animal cell models are used to achieve or determine a desirable concentration and total dosing range and route of administration. Such information is used to determine useful doses and routes for administration in humans.

A therapeutically effective dose refers to that amount of active agent that modulates or ameliorates the symptoms or condition of an ossification disease, e.g., prevents or reduces trans-differentiation of stem cells. Therapeutic efficacy and toxicity of active agents is determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it is expressed as the ratio, LD50/ED50. Pharmaceutical compositions which exhibit large therapeutic indices are preferred. The data obtained from cell culture assays and animal studies are used in formulating a range of dosage for human use.

The daily dosage of the products may be varied over a wide range, such as from 0.001 to 100 mg per adult human per day. For bolus or drip administration, the compositions are preferably provided in the form of a solution containing 0.001, 0.01, 0.05, 0.1, 0.5, 1.0, 2.5, 5.0, 10.0, 15.0, 25.0, 50.0, 100.0, 250.0, or 500.0 micrograms of the active ingredient for the symptomatic adjustment of the dosage to the patient to be treated.

A unit dose typically contains from about 0.001 micrograms to about 500 micrograms of the active ingredient, preferably from about 0.1 micrograms to about 100 micrograms of active ingredient, more preferably from about 1.0 micrograms to about 10 micrograms of active ingredient. An effective amount of the drug is ordinarily supplied at a dosage level of from about 0.0001 mg/kg to about 25 mg/kg of body weight per day. For example, the range is from about 0.001 to 10 mg/kg of body weight per day, or from about 0.001 mg/kg to 1 mg/kg of body weight per day. The compositions may be administered on a regimen of for example, one to four or more times per day.

Administration of a source of expression of a modulator is administration of a dose of a vector, such that the dose contains at least about 5000 to 10⁸ vector particles per dose.

### Administration of Pharmaceutical Compositions

As formulated with an appropriate pharmaceutically acceptable carrier in a desired dosage, the pharmaceutical composition provided herein is administered to humans and other mammals to the affected tissue or surgical site such as intramuscular, intravenous, and subcutaneous.

Liquid dosage forms for ocular, oral, or other systemic administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the ocular, oral, or other systemically-delivered compositions can also include adjuvants such as wetting agents, and emulsifying and suspending agents.

Dosage forms for peri- or post-surgical administration of an inventive pharmaceutical composition include ointments, pastes, creams, lotions, gels, powders, solutions, sprays, or patches. The active agent is admixed under sterile conditions with a pharmaceutically acceptable carrier and any needed preservatives or buffers as may be required. Administration may be therapeutic or it may be prophylactic. The invention includes surgical devices or products which contain disclosed compositions (e.g., gauze bandages or strips), and methods of making or using such devices or products. These devices may be coated with, impregnated with, bonded to or otherwise treated with a composition as described herein.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

The invention having now been fully described, it is further illustrated by the following examples and claims, which are illustrative and are not meant to be further limiting.

A portion of this work has been submitted to PLos-One as a manuscript entitled, “A molecular switch for chondrogenic differentiation of muscle progenitor cells”, co-authored by Dana M. Cairns, Renjing Liu, Manpreet Sen, James P. Canner, Aaron Schindeler, David G. Little, and Li Zeng, which is hereby incorporated by reference herein in its entirety.

The compositions, methods and kits now having been described are exemplified by the following examples and claims, which are exemplary only and are not intended to be construed as further limiting. The contents of all of the references cited are hereby incorporated herein by reference.

### EXAMPLES

### Example 1

### Isolation of Satellite Cells

Chicken eggs were purchased from Hy-line Inc., Pennsylvania. Satellite cells were isolated from day 17 chicken pre-hatch embryos [22]. Pectoral muscles were dissected, placed into sterile phosphate buffered saline (PBS) with penicillin/streptomycin, and then minced. Ground muscle was placed in a centrifuge tube and digested with pronase (1 mg/ml in PBS) in a 37° C. water bath with agitation for 40 minutes (agitation every ten minutes). Tubes were centrifuged at 3000 revolutions per minute (rpm) for four minutes. The supernatant was discarded and replaced with PBS then vortexed briefly. Tubes were then centrifuged at 1000 rpm for ten minutes three times, and the supernatants from each cycle were saved and pooled into new sterile 50 milliliter (ml) centrifuge tubes. Supernatants were then passed through a 40 micrometer (μm, micron) cell strainer (BD Biosciences, San Jose, Calif.). The cell strained supernatants were then centrifuged at 3000 rpm for six minutes and the resulting supernatants were discarded. The cell pellet was re-suspended in medium including Dulbecco's Modified Eagle Medium (DMEM; Invitrogen, CA), 10% fetal bovine serum (FBS; Hyclone Laboratories Inc., Skokie, Ill.) and 1% penicillin/streptomycin (Invitrogen Inc., Grand Island, N.Y.). The cells were then plated on tissue culture plates. Plates were incubated for 24 hours in a humidified incubator at 37° C. with 5% CO₂, and then washed with sterile PBS to remove non-adherent cells. Freshly isolated cells were confirmed to be positive for satellite cell specific markers, Pax3 and Pax7, before subsequent experiments were conducted.

### Example 2

### Cell Culture

Satellite cells were cultured in regular culture medium and/or chondrogenic induction medium. Regular culture medium included DMEM with 10% FBS (Hyclone, Logan, Utah) and 1% antibiotic/mycotic (Invitrogen, CA). The chondrogenic induction, satellite cells were plated as high density micromass cultures in the presence of chondrogenic induction media, which included DMEM (Invitrogen Inc.) supplemented with 1.0 mg/ml recombinant human insulin, 0.55 mg/ml human transferring (substantially iron-free), and 0.5 μg/ml (microgram/ml) sodium selenate (ITS, catalog number 12521 Sigma-Aldrich, St. Louis, Mo.), 0.1 mM ascorbic acid (Sigma-Aldrich), human serum albumin (HSA, Sigma-Aldrich), 10⁻⁷ molar dexamethasone, 10 ng/ml TGFβ3 (R&D Systems, Minneapolis, Minn.) or BMP2 (R&D Systems) [23] [24]. Cells were split and re-suspended (10⁵ cells/10 μl droplet). The cells were then pipetted onto a plate and allowed to adhere in a 37° C., 5% CO₂ incubator for approximately one hour before the addition of chondrogenic media. Cells were grown for five days and then were analyzed by histological and qRT-PCR analyses,

### Example 3

### Virus Production and Delivery of Satellite Cells

Avian-specific retroviruses (RCAS) were generated by transfecting chick embryonic fibroblasts (CEF) with retroviral vector constructs encoding for the following genes: GFP, Nkx3.2HA, Sox9V5, Alkaline phosphatase (AP), Pax3HA, Nkx3.2ΔC-HA (deletion of C-terminus from aa219-278), or Nkx3.2ΔC-VP16 [25,26,27]. The viral supernatant was concentrated by ultracentrifugation at 21,000 rpm for two hours. The centrifuged materials were then titered by directly visualizing GFP expression (in the case of RCAS-GFP) or indirect immunocytochemistry using anti-GAG antibody (which recognizes the viral coat protein GAG). Viruses with titers of at least 10⁸ particles/ml were used in all satellite cell cultures. Viruses of different coat proteins A- or B- were used for co-delivery examples. Retroviral delivery by infection of satellite cells was carried out by directly adding concentrated virus into growing cell cultures. High levels of expression were detectable 48 hours after viral infection. The cells were then split into samples and used in subsequent examples described herein.

### Example 4

### Luciferase Assays and Analysis

A murine Pax3 promoter sequence (1.5 kb) [28] was cloned into SmaI and NheI sites of the pGL3 luciferase vector (Promega Inc.; Madison, Wis.) for the synthesis of the luciferase construct. Satellite cells were transfected with pGL3-Pax3 promoter construct or pGL3 control using Fugene6 according to the manufacturer's protocol. Cells were processed after 48 hours using the Luciferase Assay System (Promega Inc.). Cells were thoroughly disrupted with lysis buffer using a freeze-thaw cycle. Supernatants were added to the luciferase assay reagent in a 96 well plate, then analyzed using a 1450 Microbeta Wallac Trilux plate reading luminescence counter (Perkin Elmer, MA).

### Example 5

### Histological Analyses

Samples were fixed with 4% paraformaldehyde (Sigma-Aldrich). For Alcian blue staining, cryosections of satellite cell micromass cultures were pre-washed with 0.1N HCl then incubated with 1% (w/v) alcian blue (Sigma-Aldrich) overnight. The sections were then repeatedly washed with 0.1N HCl. Hematoxylin and eosin (H&E; Sigma-Aldrich) staining was performed according to standard protocol on cryosectioned mouse tissues. Staining for heat-inactivated alkaline phosphatase (HI-AP) on serial cryosectioned mouse tissue was performed by incubating the slides at 75° C. for 50 minutes to eliminate endogenous alkaline phosphatase activity. The sections were then contacted with p-nitro-blue-tetrazolium (NBT; 100 mg/ml in 70% dimethyl formamide) and 5-bromo-4-chloro-3-indoyl phosphate (BCIP; 50 mg/ml dimethyl formamide) (Invitrogen, CA).

The following primary antibodies were used for immunocytochemical analysis of the samples: mouse anti-collagen II; rabbit anti-collagen II (Abcam Inc.; Cambridge, Mass.), rabbit anti-Sox9 (Chemicon International Inc.; Billerica Mass.), mouse anti-Pax3 (Developmental Studies Hybridoma Bank (DSHB); Iowa City, Iowa), mouse anti-Pax7 (DSHB), mouse anti-myosin heavy chain (DSHB; catalog number MF20), mouse anti-GAG (DSHB), rabbit anti-HA (Sigma-Aldrich); rabbit anti-V5 (Sigma-Aldrich); rabbit anti-VP16 (Abcam Inc.). For immunohistochemistry of mouse tissues, cryosections were first subject to antigen retrieval by treating slides with 1% sodium dodecyl sulfate (SDS) in PBS for five min at room temperature prior to subsequent staining steps. Unless indicated, no antigen retrieval was used for all other immunocytochemistry of cell culture. Samples were first blocked with PBS with 0.1% Triton-X (Sigma) and 6% goat serum (Sigma-Aldrich), and then incubated with primary antibodies overnight. Samples were repeatedly washed with PBS with 0.1% Tween (PBST), and then incubated with secondary antibodies. For immunofluorescent staining, secondary antibodies used were conjugated with Alexa 488 (green) or 594 (red) (Invitrogen, CA). Cultures were counterstained with DAPI (Invitrogen, CA). Secondary antibody was conjugated with biotin for colorimetric immunostaining, and the signal was amplified using the Vectastain Elite ABC kit (Vector Laboratories; Burlingame, Calif.) and developed using DAB-peroxidase (Sigma-Aldrich).

### Example 6

### Reverse Transcriptase-Polymerase Chain Reaction (RT-PCR) Analysis

RNA was isolated from all cell cultures using the RNeasy mini-kit (Qiagen Inc.; Chatsworth, Calif.). The Qiagen MicroKit (Chatsworth, Calif.) was used for RNA samples isolated from mouse tissue cryosections using laser capture microscopy (LCM). Murine leukemia virus reverse transcriptase (MLV-RT; Invitrogen, CA) was used according to a standard protocol to generate cDNA. An iQ5 Real-Time PCR Detection System (BioRad Inc.; Hercules, Calif.) was used for Quantitative PCRs. PCR analyses of in vitro experiments were normalized to glyceraldehyde 3-phosphate dehydrogenase (GAPDH). PCR analyses from in vivo mouse LCM samples were normalized to the 18S RNA. Nucleic acid sequences and sequence identification numbers for primers used for PCR are listed in Table 3. Nucleic acid sequences, amino acid sequences, and corresponding sequence identification numbers for proteins encoded by the genes herein are shown in Table 4.

### Example 7

### Microscopy

Bright-field and fluorescent images from histological and immunocytochemistry analyses were collected with the Olympus IX71 inverted microscope using an Olympus DP70 digital camera and associated software (Olympus Inc; Center Valley, Pa.). Laser capture microscopy (LCM) was performed using the Arcturus PixCell IIe system (Tufts Imaging Facility, Center for Neuroscience Research) using the established protocol [29,30]. Cryosectioned tissues were dehydrated and were overlaid with a thermoplastic membrane, which was mounted on an optically transparent cap (Arcturus Macro LCM caps, Applied Biosystem, CA). Target tissues were identified by comparisons with serial sections that were stained with heat-inactivated alkaline phosphatase (HI-AP). Target cells were captured by focal melting of the membrane after laser activation, then the captured tissue was immersed in a denaturation solution and was subsequently subject to RNA isolation.

TABLE 3
PCR primers in DNA amplification

| | Gene | Nucleic acid | Nucleic acid |
|---|---|---|---|
| | Accession | Forwad sequence | Reverse sequence |
| Species | number | (SEQ ID NO) | (SEQ ID NO) |
| chicken | GAPDH | 5′- CCT GCT GCC TAG | 5′- CAG ATC AGT TTC TAT |
| | NM_204305.1 | GGA AGC -3′ | CAG CCT CT -3′ |
| | | (SEQ ID NO: 1) | (SEQ ID NO: 2) |
| chicken | collagen I1 | 5′- GCA CAA CTT CTG | 5′- TCA CAC CTG CCA GAT |
| | NM_204426.1 | CAC TGA ACG GAT -3′ | TGA TTC CCA -3′ |
| | | (SEQ 1D NO: 3) | (SEQ ID NO: 4) |
| chicken | aggrecan | 5′- AGT GAC AAC CCA | 5′- AGA AGC GCT CCC ACC |
| | XM_001232949.1 | GTC AGT TGC AGA -3′ | AAA GTC TAT -3′ |
| | | (SEQ ID NO: 5) | (SEQ ID NO: 6) |
| chicken | Nkx3.2 | 5′- TGC AGC CCT CCT | 5′- CGG GCT GCT TAC ACA |
| | NM_204137.1 | CAC AAG TGT AAT -3′ | CAT TCA CAA -3′ |
| | | (SEQ ID NO: 7) | (SEQ ID NO: 8) |
| chicken | Sox9 | 5′- GTC TCT GCC GGC TTT | 5′- TGC GAG AAA GCG GCA |
| | NM_204281.1 | ACT TCT TGT -3′ | CAG GG -3′ |
| | | (SEQ ID NO: 9) | (SEQ ID NO: 10) |
| chicken | Pax3 | 5′- TTC AGG TTT GGT TTA | 5′- TAC TGC TTG GAT CAG |
| | NM_204269.1 | GCA ACC GCC -3′ | ACA CGG CTT -3′ |
| | | (SEQ ID NO: 11) | (SEQ ID NO: 12) |
| chicken | Pax7 | 5′- AGC COT GTG CTA | 5′- TTC CTC TTC AAA GGC |
| | NM_205065.1 | CGC ATC AAA TTC -3′ | AGG TCT GGT -3′ |
| | | (SEQ ID NO: 13) | (SEQ ID NO: 14) |
| chicken | MyoD | 5′- ACG ACA GCA OCT | 5′- TCT CCA CAA TGC TTG |
| | NM_204214.1 | ACT ACA CGG AAT -3′ | AGA GGC AGT -3′ |
| | | (SEQ ID NO: 15) | (SEQ ID NO: 16) |
| chicken | Myogenin | 5′- TGA AAC CGC CCA | 5′- CGA AGA GCA ACT TGG |
| | NM_204184.1 | AAT CCT TTC CCA -3′ | AAA CAG CCA -3′ |
| | | (SEQ ID NO: 17) | (SEQ ID NO: 18) |
| chicken | myosin heavy | 5′- GCA GAA TTT CAG | 5′- TGA CTC GTT GCA GGT |
| | chain | AAG ATG CGC CGT -3′ | TGT CGA TCT -3′ |
| | NM_204228.1 | (SEQ ID NO: 19) | (SEQ ID NO: 20) |
| mouse | 18S | 5′- TCA ACT TTC GAT | 5′- TCC TTG GAT GTG GTA |
| | NR_003278.2 | GGT AGT CGC CGT -3′ | GCC GTT TCT -3′ |
| | | (SEQ ID NO: 21) | (SEQ ID NO: 22) |
| mouse | Collagen II | 5′- ACA TAG GGC CTG | 5′- TGA CTG CGG TTG GAA |
| | NM_001113515.2 | TCT GCT TCT TGT -3′ | AGT GTT TGG -3′ |
| | | (SEQ ID NO: 23) | (SEQ ID NO: 24) |
| mouse | Nkx3.2 | 5′- TCA GAA CCG TCG | 5′- CAG CAC CTT TAC GGC |
| | NM_007524.3 | CTA CAA GAC CAA -3′ | CAC TTT CTT -3′ |
| | | (SEQ ID NO: 25) | (SEQ ID NO: 26) |
| mouse | Sox9 | 5′- AGG TTT CAG ATG | 5′- ACA TAC AGT CCA GGC |
| | NM_011448.4 | CAG TGA GGA GCA -3′ | AGA CCC AAA -3′ |
| | | (SEQ ID NO: 27) | (SEQ ID NO: 28) |
| mouse | Pax3 | 5′- TAC CAG CCC ACG | 5′- TTT GGT GTA CAG TGC |
| | NM_008781.4 | TCT ATT CCA CAA -3′ | TCG GAG GAA -3′ |
| | | (SEQ 1D NO: 29) | (SEQ ID NO: 30) |
| mouse | Pax7 | 5′- TTC AAA GGA GGA | 5′- TGT GGA GGA GGA TGC |
| | NM_011039.2 | GAC TGT TGG GCT -3′ | ATT TGG TCT -3′ |
| | | (SEQ ID NO: 31) | (SEQ ID NO: 32) |
| mouse | Myosin Heavy | 5′- AGG CTT ACA AGC | 5′- ACC GCA TGG CAT ACT |
| | Chain | AAA TGG CAA GGG -3′ | TAG CAG AGA -3′ |
| | (MHC) | (SEQ ID NO: 33) | (SEQ ID NO: 34) |
| | X57377.1 | | |

TABLE 4
Nucleic acid sequences and amino acid sequences for proteins encoded
by genes listed in Table 3.

| | Molecule | Nucleic acid | Amino acid |
|---|---|---|---|
| Species | Accession number | SEQ ID NO: | SEQ ID NO: |
| chicken | GAPDH | 35 | 36 |
| | NM_204305.1 | | |
| chicken | collagen II | 37 | 38 |
| | NM_204426.1 | | |
| chicken | aggrecan | 39 | 40 |
| | XM_001232949.1 | | |
| chicken | Nkx3.2 | 41 | 42 |
| | NM_204137.1 | | |
| chicken | Sox9 | 43 | 44 |
| | NM_204281.1 | | |
| chicken | Pax3 | 45 | 46 |
| | NM_204269.1 | | |
| chicken | Pax7 | 47 | 48 |
| | NM_205065.1 | | |
| chicken | MyoD | 49 | 50 |
| | NM_204214.1 | | |
| chicken | Myogenin | 51 | 52 |
| | NM_204184.1 | | |
| chicken | myosin heavy chain | 53 | 54 |
| | NM_204228.1 | | |
| mouse | 18S | 55 | — |
| | NR_003278.2 | | |
| mouse | Collagen II | 57 | 58 |
| | NM_001113515.2 | | |
| mouse | Nkx3.2 | 59 | 60 |
| | NM_007524.3 | | |
| mouse | Sox9 | 61 | 62 |
| | NM_011448.4 | | |
| mouse | Pax3 | 63 | 64 |
| | NM_008781.4 | | |
| mouse | Pax7 | 65 | 66 |
| | NM_011039.2 | | |
| mouse | Myosin Heavy Chain | 67 | 68 |
| | X57377.1 | | |

### Example 8

### Fracture Creation in MyoD-cre Z/AP Labeled Mice

MyoD-cre Z/AP reporter mice were bred by the crossing of the MyoD-cre [4] and Z/AP [31] lines. The MyoD-Cre mouse line was obtained from the University of Connecticut, Storrs, USA). The Z/A line was supplied by the Children's Medical Research Institute (Westmead, NSW, Australia) and the Samuel Lunenfeld Research Institute (Toronto, Ontario). The cross strain labels all MyoD(+) lineage cells to permanently express the heat-resistant human placental alkaline phosphatase (hPLAP). Midshaft tibial fractures were generated in anaesthetized MyoD-cre Z/AP mice and littermate controls by manual three point using a previously published model [32]. Tissue specimens were harvested from mice at one week endpoint were used for enzymatic and immunohistochemical staining. Animal experimentation was approved by the CHW/CMRI Animal Ethics Committee (K248) and the Westmead Hospital Animal Ethics Committee (4102).

### Example 9

### Statistical Analysis

For statistical analysis, the mean and standard deviation were calculated. Statistically significant differences (i.e., p<0.05) were determined by one-factor analysis of variance (ANOVA) with post hoc Tukey test using the statistics software SYSTAT12 (Systat, Chicago, Ill., USA).

### Example 10

### Identification and Analysis of Muscle Satellite Cells

Muscle satellite cells were isolated from the pectoralis muscles of embryonic day 17 chicken embryos. Tissues were minced and were digested with enzymes. Mononuclear satellite cells were specifically isolated by differential centrifuging the digested material. The cells were identified and confirmed as muscle stem cells by immunoassay and protein analyses.

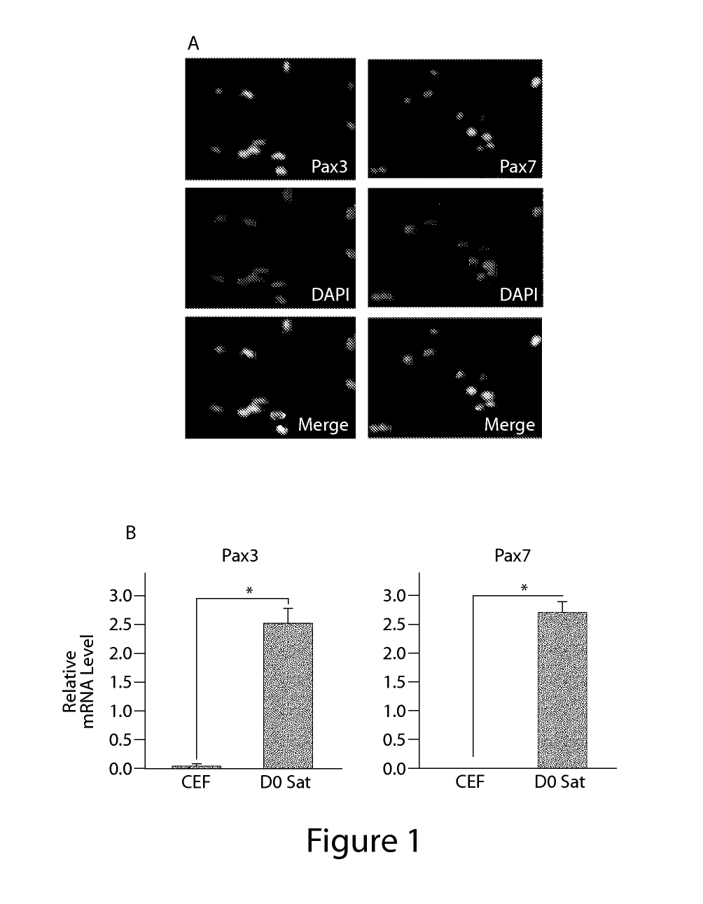

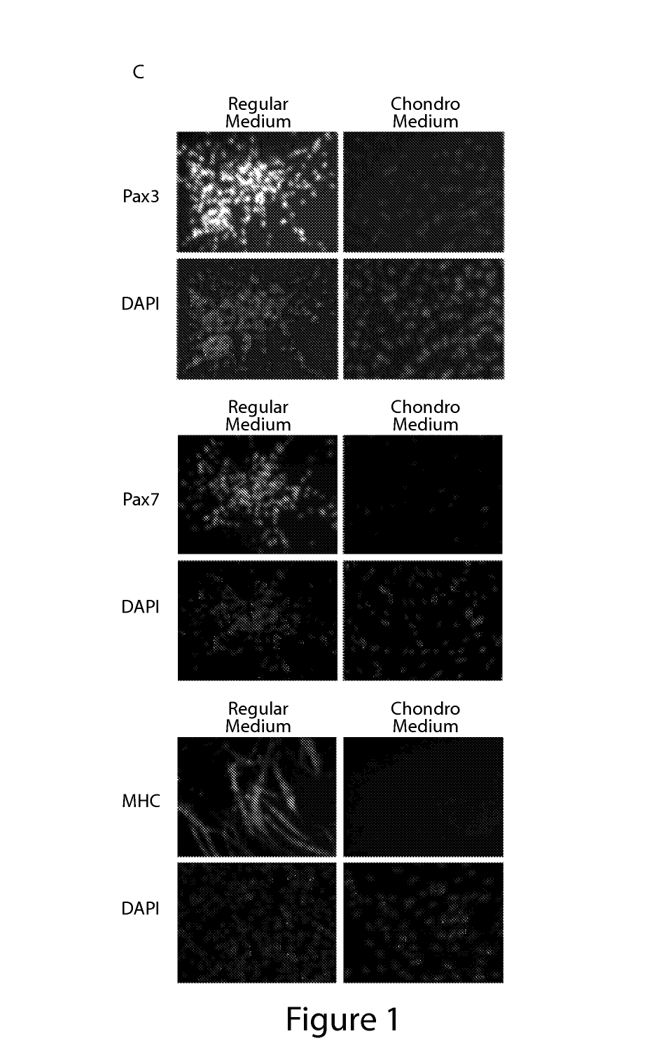

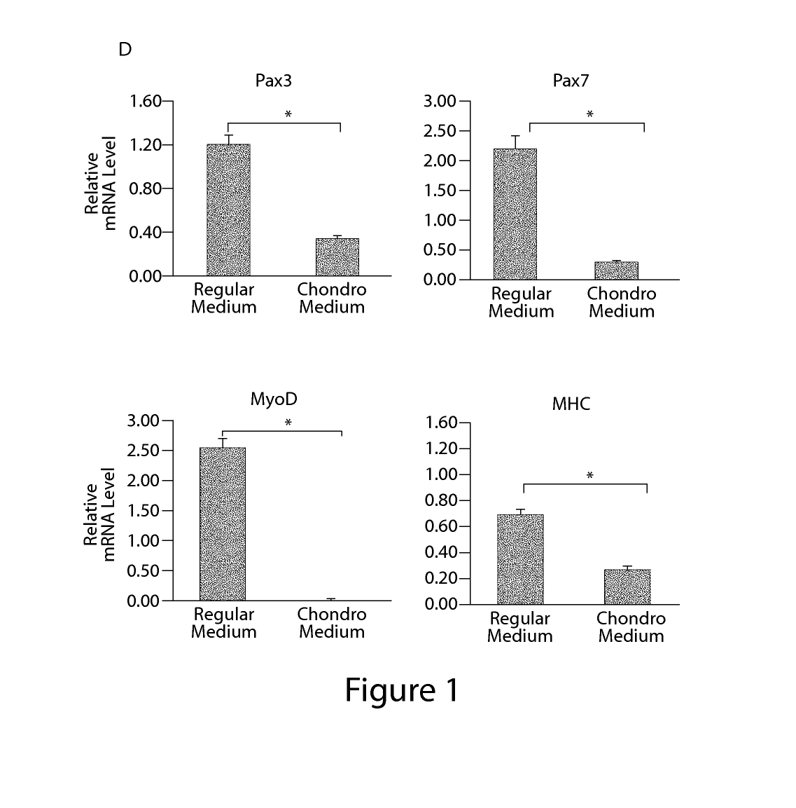

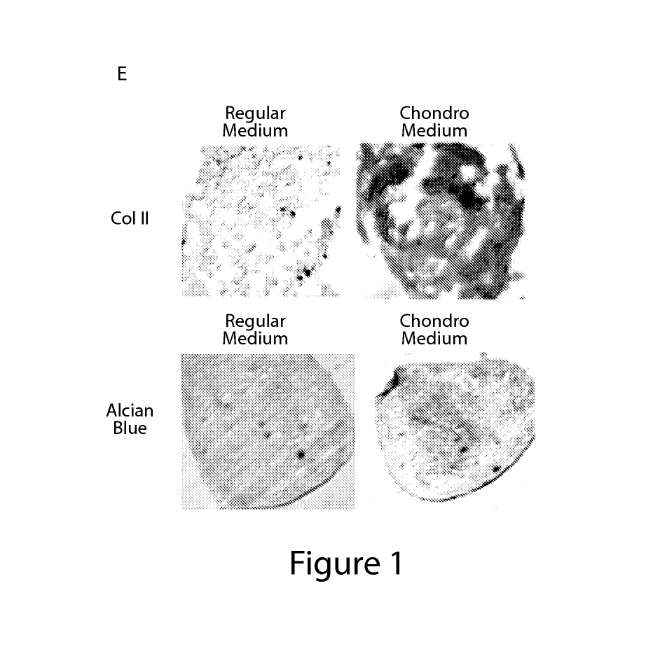

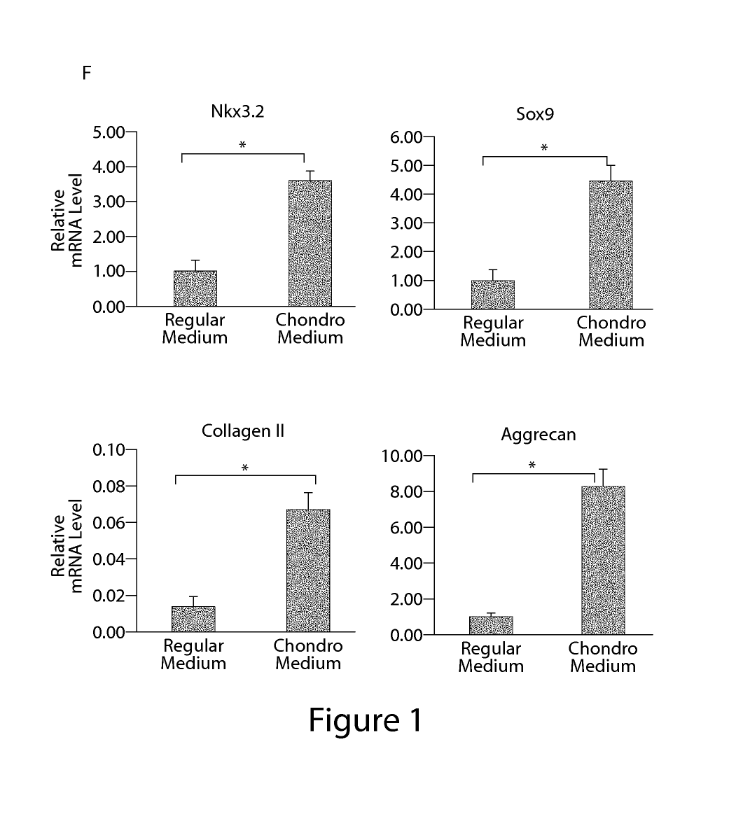

Muscle satellite cells were isolated from the pectoralis muscles of embryonic day 17 chicken embryos. Isolated muscle cells differentiate into muscle cells that are phenotypically similar to adult muscle cells [33]. Tissues were minced and were digested with Pronase (Roche Inc.: Indianapolis, Ind.) which is a commercially available mixture of proteinases isolated from the extracellular fluid of Streptomyces griseus. Mononuclear satellite cells were specifically isolated by differential centrifuging the digested material. Muscle satellite cells analyzed by immunocytochemistry analysis at day zero (D0) as shown in FIG. 1 panel A. The presence of the muscle satellite cells was also determined using qRT-PCR analysis, which identified strong expression of Pax3 and Pax7 in the muscle satellite cells (FIG. 1 panel B). Control chicken embryonic fibroblast cells that do not express Pax3 and Pax7 were analyzed also and data showed that these cells did not show expression of Pax3 and Pax7 (FIG. 1 panel B). The cells were identified and confirmed as muscle stem cells by immunoassay and protein analysis and were found to be greater than 95% positive for Pax3 and Pax7, specific markers for muscle stem cells.

The muscle satellite cells were then cultured in three-dimensional (3D) micromass cultures in the presence of the standard chondrogenic (induction) medium containing TGFβ3 [23,24], or regular/control growth medium. The 3D culture system differentiates embryonic progenitor cells or bone marrow-derived mesenchymal stem cells into cartilage [23,24,34]. Immunocytochemistry and RT-PCR analyses showed that culturing muscle satellite cells in chondrogenic medium resulted in a dramatic reduction of expression of each of Pax3 and Pax7, myoblast marker MyoD, and differentiated myocyte marker myosin heavy chain (MHC). See FIG. 1 panels C and D.

Surprisingly, immunocytochemistry data showed that culturing/contacting the muscle satellite cell micromass with chondrogenic medium resulted in greater induction and expression of cartilage-specific protein collagen II compared to culturing the micromass in control growth medium (FIG. 1 panel F left photographs). Alcian blue staining showed increased expression of glycosaminoglycans in muscle satellite cell micromass cultures contacted with chondrogenic medium in contrast to control growth medium. (FIG. 1 panel E left photograph and right photograph). Analysis using qRT-PCR showed that contacting the muscle satellite cells micromass with chondrogenic induction medium produced increased expression of transcription factors Nkx3.2 and Sox9, and cartilage markers cartilage matrix markers collagen II and aggrecan compared to cells contacted with control medium (FIG. 1 panel F).

Another sample of muscle satellite cells in a 3D micromass was cultured in a chondrogenic medium containing BMP2. TGFβ3 was omitted. Contacting muscle satellite cells with BMP2-containing chondrogenic medium resulted in increased expression of transcription factors Nkx3.2 and Sox9, and cartilage markers cartilage matrix markers collagen II and aggrecan, compared to cells contacted with control medium. Thus, similar results were observed for muscle satellite cells contacted with a chondrogenic medium containing TGFβ3 and muscle satellite cells contacted with chondrogenic induction medium containing BMP2. Data clearly demonstrated that muscle satellite cells have the ability to form a cartilage phenotype in vitro at the expense of the default muscle cell fate.

### Example 11

### Viral Vector Constructs

To determine the effect of Nkx3.2, Sox9 and Pax3 on trans-differentiation of muscle stem cells, avian retrovirus (RCAS) vectors having nucleic acids that encode Nkx3.2, Sox9, and gene fusions of Nkx3.2 were constructed. Methods of constructing vectors carrying genes encoding Nkx3.2, Sox9 and Pax3 are described herein and are shown in Zeng, L. et al. 2002 Genes & Development 16: 1990-2005. Nucleic acid sequences for primers used for PCR are listed in Table 3. Nucleic acid sequences, amino acid sequences and sequence identification numbers for proteins synthesized herein are shown in Table 4.

### Example 12

### Pax3 Inhibited the Adoption of Cartilage Cell Fate by Muscle Satellite Cells

intracellular mechanisms and factors on the chondrogenic differentiation of satellite cells were investigated. Muscle marker Pax3 expression was strongly downregulated in muscle satellite cells cultured in chondrogenic medium (see FIG. 1 panels C and D).

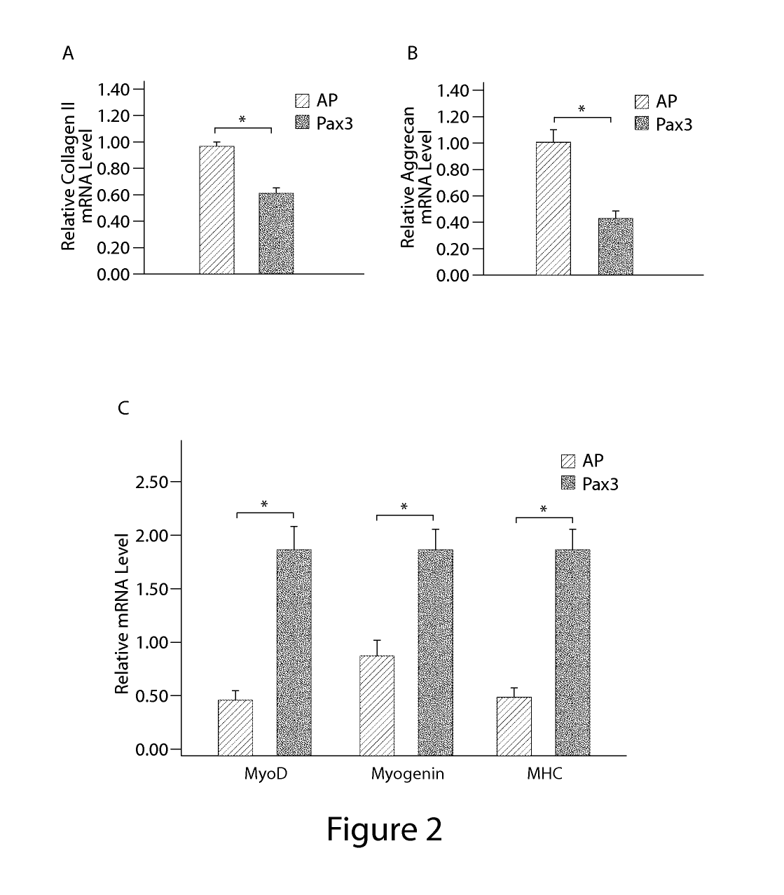

To determine whether Pax3 negatively regulated the differentiation of satellite cells to chondrocytes, muscle satellite cells were contacted with a Pax3-expressing retrovirus vector or a control vector encoding alkaline phosphatase (AP). The virus-contacted cells were then cultured in chondrogenic (induction) medium in a 3D micromass. It was observed that forced expression of Pax3 in muscle satellite cells using a vector resulted in a significant decrease in expression of cartilage markers collagen II and aggrecan compared to cells contacted with the control vector encoding alkaline phosphatase (FIG. 2 panels A and B). Relative mRNA collagen or aggrecan levels were normalized against cells contacted with a vector encoding GADPH.

It was observed also that contacting muscle satellite cells with a vector encoding Pax3 increased the expression of muscle markers MyoD, myogenin, and MHC by approximately two-fold (FIG. 2 panel C). Therefore, these data showed that Pax3 inhibited chondrogenesis in muscle satellite cells, and inhibition was associated with decreasing expression of cartilage markers and increasing expression of muscle cell markers. Inhibition of Pax3 was required for muscle satellite cells to differentiate into chondrocytes.

### Example 13

### Modulating Trans-Differentiation of Muscle Satellite Cells to Mature Muscle Cells Using Nkx3.2 or Sox9

Transcriptions factors induced in muscle satellite cells by chondrogenic medium were investigated to determine whether these transcriptions factors specifically inhibit the default muscle fate of muscle satellite cells.

Sox9 and Nkx3.2 are factors induced by TGFβ-containing chondrogenic medium (FIG. 1 panel F). These transcription factors have been identified in cartilage formation during embryogenesis [35,39,40]. However, it has not been determined whether these transcription factors specifically influence chondrogenic differentiation of muscle satellite cells.

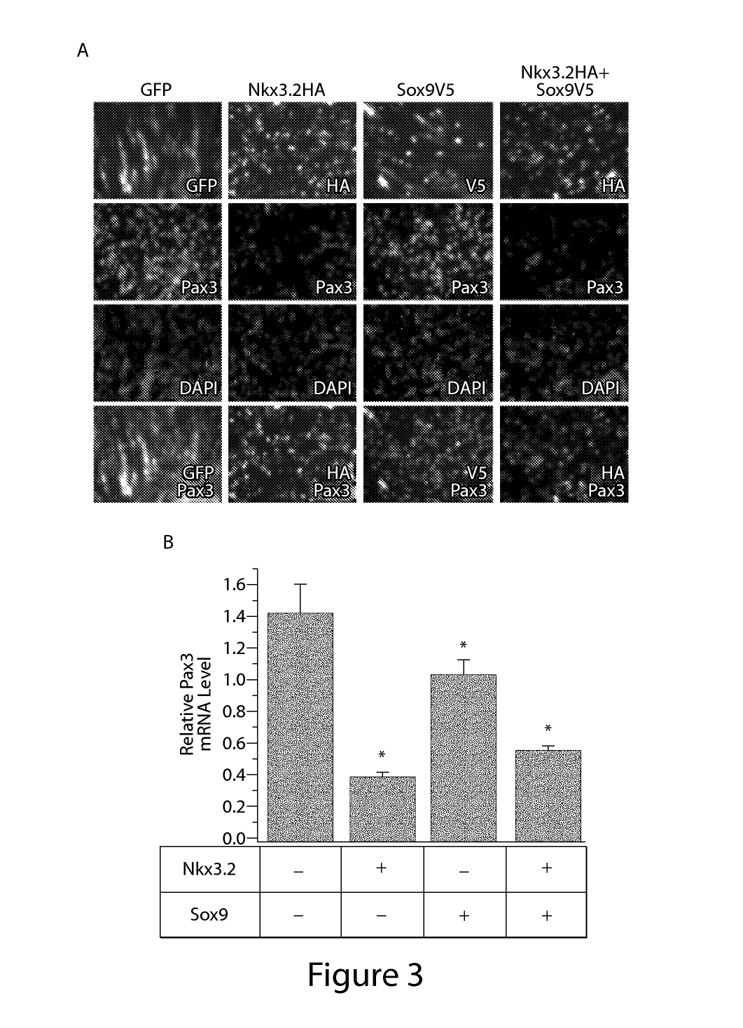

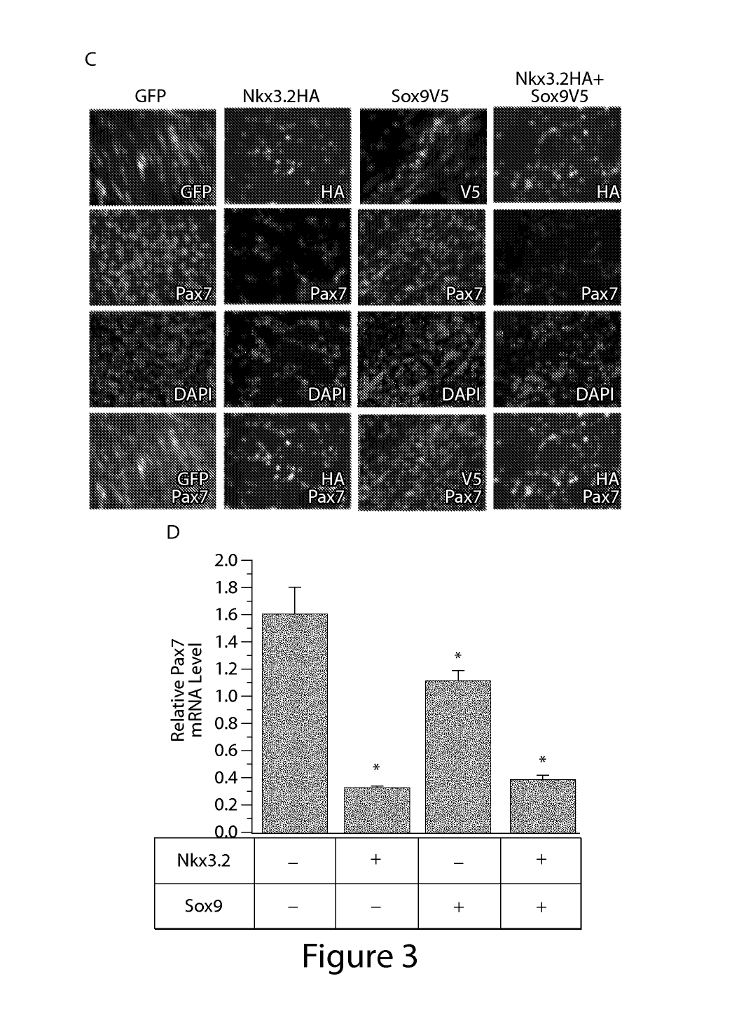

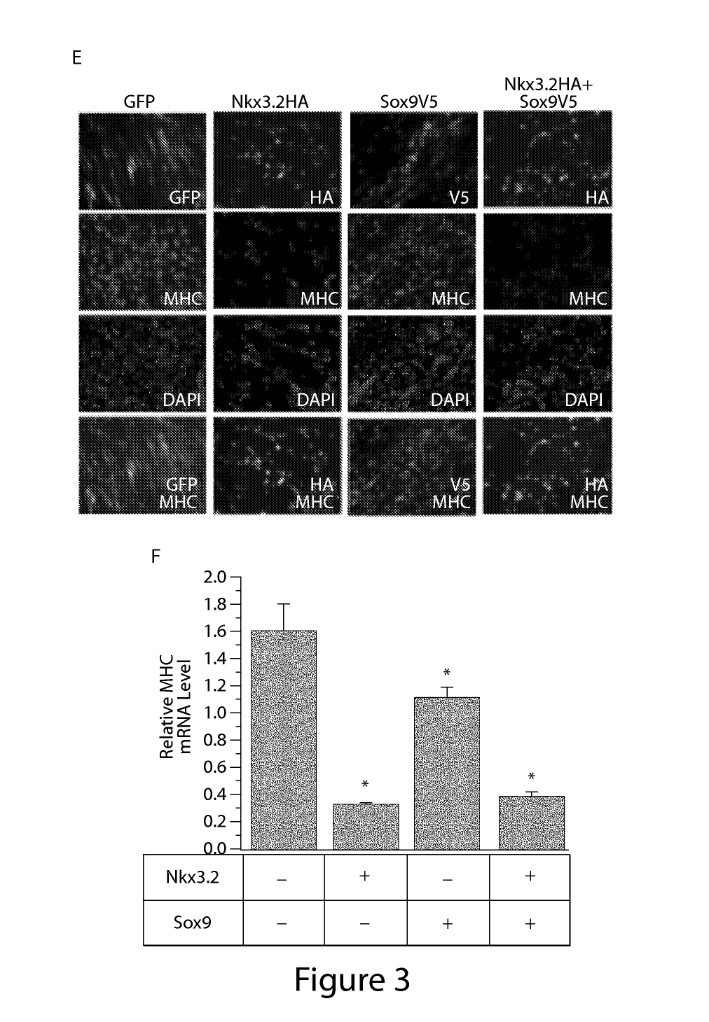

Muscle satellite cells were contacted with a retrovirus vector encoding Nkx3.2, and protein expression was analyzed using immunostaining and qRT-PCRT. Immunostaining and qRT-PCR analyses showed that the vector encoding Nkx3.2 strongly inhibited Pax3 expression in the muscle satellite cells (FIG. 3 panels A and B respectively).

Muscle satellite cells contacted with a vector encoding Sox9 showed weak downregulation of Pax3 expression, indicating that Nkx3.2 is a more potent inhibitor of muscle cell fate in satellite cells than Sox9, as shown in a comparison of FIG. 3 panels A and B. Furthermore, Nkx3.2 inhibited the expression in muscle satellite cells of muscle markers Pax7 and myosin heavy chain (MHC), which is a marker for differentiated myocytes (FIG. 3 panels C-F). The muscle satellite cells upon isolation expressed a higher level of Pax3 and Pax7 than MHC. Data showed a surprisingly dramatic reduction in the expression of MHC (FIG. 3). Muscle gene expression was not as clearly visible in the less quantitative immunocytochemistry analysis of the muscle satellite cells contacted with Sox9 (FIG. 3 panels A, C and E).

Muscle gene expression and cartilage gene expression were further evaluated using analytical techniques qRT-PCR and immunocytochemistry. These techniques were used to measure efficacy of transcription factors Nkx3.2 and Sox9 to modulate trans-differentiation of muscle satellite cells. Three-dimensional micromass cultures containing muscle satellite cells were cultured in chondrogenic medium having transforming growth factor beta (TGF-β) or bone morphogenic protein-4 (BMP4). The chondrocyte forming medium was observed to have induced expression of cartilage markers collagen II and aggrecan, and of proteins Nkx3.2 and Sox9.

The roles of Nkx3.2 and Sox9 were further investigated using constructs encoding these proteins. Two-dimensional and three-dimensional muscle stem cell cultures were contacted with vectors carrying nucleotide sequences encoding Nkx3.2 or Sox9. Vectors carrying Nkx3.2 were observed to strongly inhibit Pax3 and Pax7 compared to vectors carrying Sox9 or control GFP. Data in FIG. 3 panel E (second row) show that cells contacted with Nkx3.2, or both Nkx3.2 and Sox9 had little or no staining of myosin heavy chain (MHC) compared to intense staining observed for cells contacted with Sox9 or GFP, showing that Nkx3.2 downwardly modulated MHC expression.

Muscle satellite cells contacted with a vector encoding Nkx3.2 only, or both with a vector encoding Nkx3.2 and a vector encoding Sox9 showed significantly reduced MHC staining on the cells compared to cells contacted with vectors encoding Sox9 or GFP (FIG. 3 panel E, compare photomicrographs in second row). Cells contacted with a vector encoding Sox9 showed strong extensive MHC staining, comparable to staining observed in cells contacted with the control vector encoding GFP (FIG. 3 panel E second row).

DAPI immunostaining of nucleic acids showed comparable amounts of DNA in cells contacted with the vectors encoding Nkx3.2, Sox9, and both Nkx3.2 and Sox9 compared to the GFP control contacted cells (FIG. 3 panels E third row). These data clearly show that inhibition of MHC expression was due to in vivo expression of transcription factors and not due to differences in DNA amounts in the cells, and that Nkx3.2 plays an important role in negatively modulating expression to suppress the muscle cell differentiation in muscle satellite cells. Further, the inhibitory effect of Nkx3.2 in muscle satellite cell differentiation is specific.

RT-PCR analysis clearly showed that the vector encoding Sox9 significantly inhibited Pax3, Pax7 and MHC expression in the muscle satellite cells (FIGS. 3B, 3D and 3F). Analysis of MHC RNA levels in FIG. 3 panel B shows that contacting muscle satellite cells with a vector that encodes Nkx3.2, or both with a vector encoding Nkx3.2 and a vector encoding Sox9 inhibited MHC expression by at least 90% in muscle satellite cells compared to cells contacted with vectors encoding Sox9 alone or neither Nkx3.2 nor Sox9 (FIG. 3 panel B). Sox9 alone inhibited MHC expression by about 35%.

Data show that combined treatment with both a vector encoding Nkx3.2 and a vector encoding Sox9 inhibited muscle cell fate similar to results for muscle satellite cells contacted with a vector encoding Nkx3.2 only.

### Example 14

### The C-Terminus of Nkx3.2 is Required for Inhibition of Muscle Cell Fate in Muscle Satellite Cells

Nkx3.2 strongly inhibited the muscle fate in satellite cells. Examples herein investigated whether this inhibitory effect was specific to a specific portion of Nkx3.2 protein by constructing Nkx3.2 gene mutants. A Nkx3.2 protein was constructed that lacks the C-terminus domain (Nkx3.2-ΔC mutant). A reverse function mutant of Nkx3.2 was constructed, the C-terminus domain of which was replaced by a VP16 constitutive activation domain (Nkx3.2ΔC-VP16). [40] [26,41]. The Nkx3.2 mutant that lacks the C-terminus domain (Nkx3.2-ΔC mutant) was generated by deleting the gene encoding 58 amino acids from the C-terminus. The reverse function mutant of Nkx3.2 was constructed by deleting the 58 amino acid C-terminus domain and inserting a VP16 constitutive activation domain (Nkx3.2ΔC-VP16).

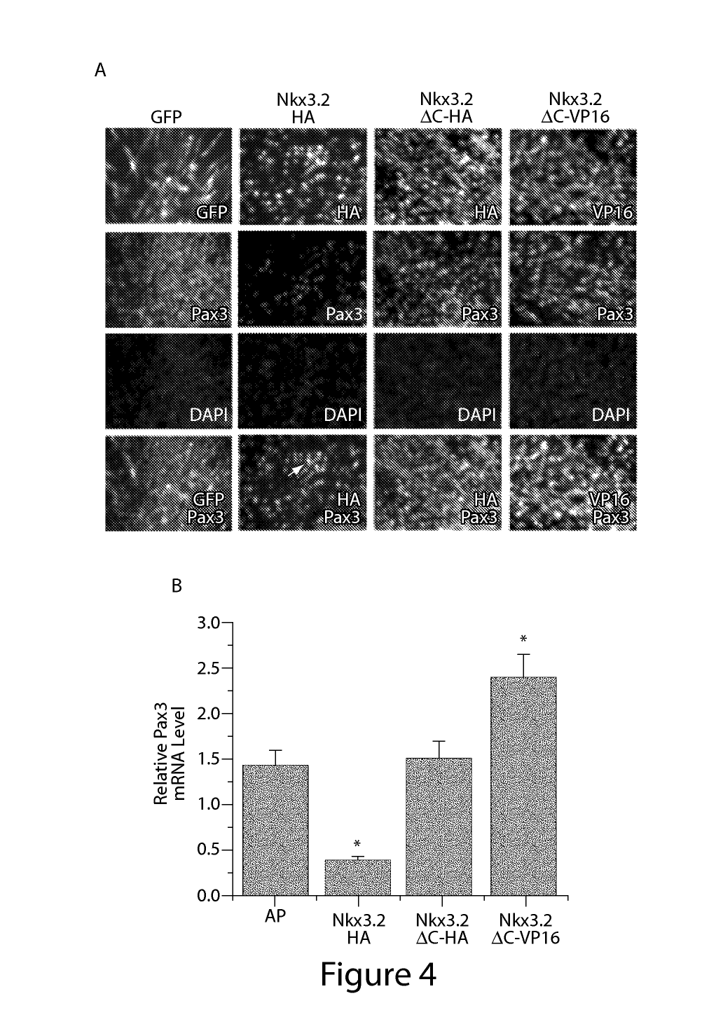

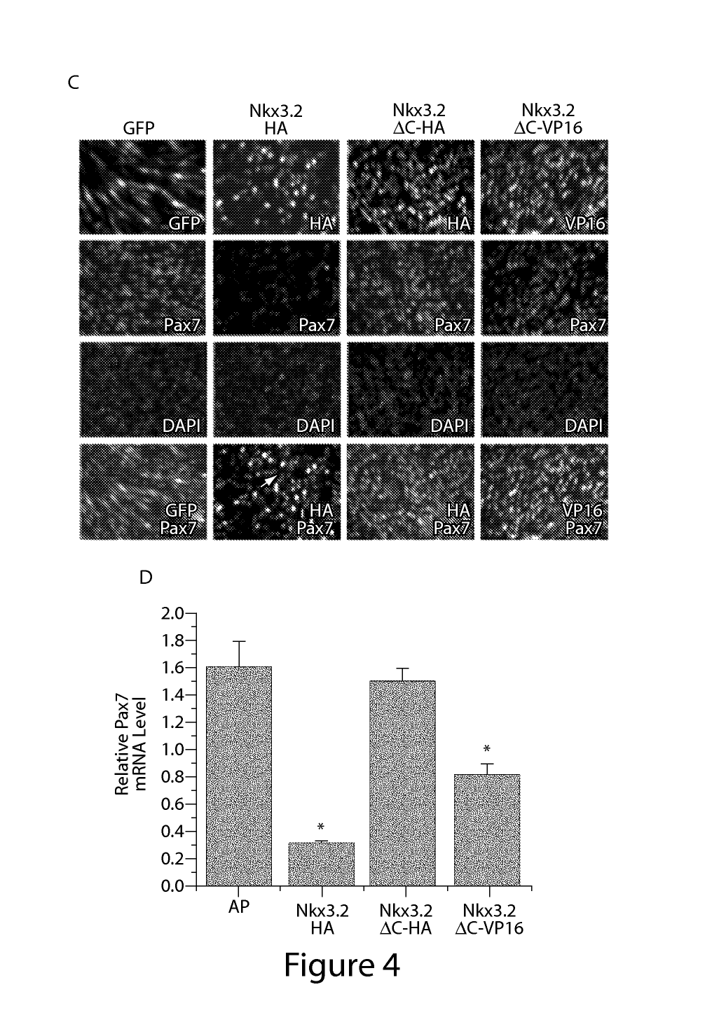

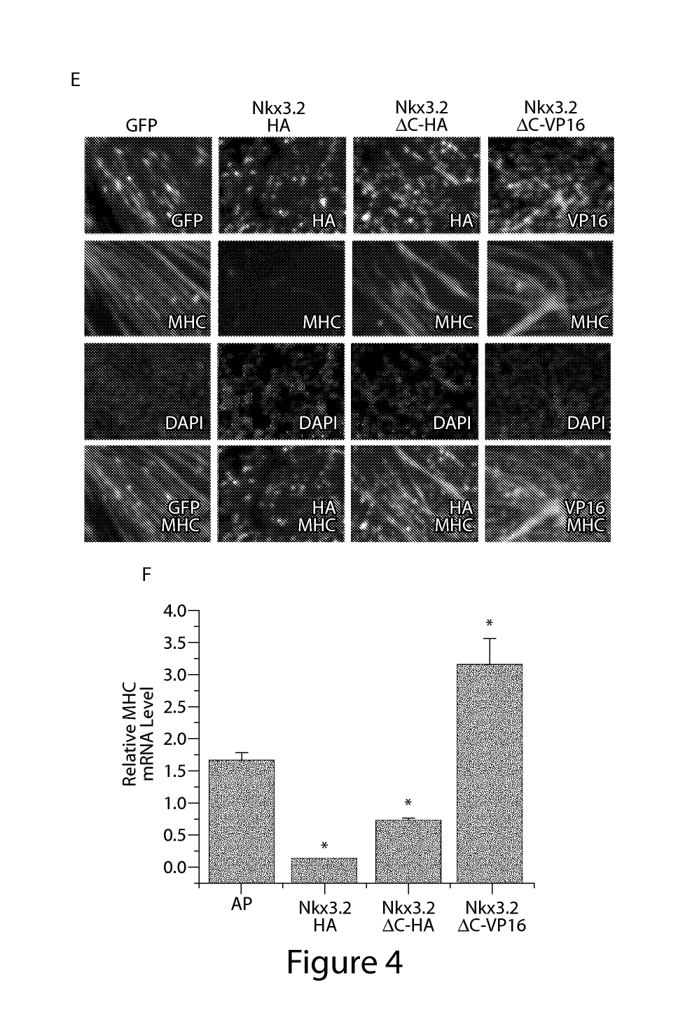

Contacting muscle satellite cells with a vector encoding full length Nkx3.2 protein was observed in examples herein to significantly reduce amount of Pax3, Pax7 and MHC expression in satellite cells (FIG. 3). Further it was observed that the Nkx3.2-C-terminus deletion mutant (Nkx3.2-ΔC-HA) did not inhibit expression of Pax3 at the protein or the mRNA level (FIG. 4 panels A and B). Most importantly, the vector encoding the reverse function mutant Nkx3.2ΔC-VP16 induced the expression of Pax3 (FIG. 4 panel B), resulting in an increase in Pax3, a muscle marker. Thus the reverse function mutant Nkx3.2ΔC-VP16 resulted in an opposite phenotype as wild type Nkx3.2 that inhibited Pax3 expression.

The Nkx3.2 gene mutants were further analyzed for their effect on other muscle markers, Pax7 and MHC. It was observed that the Nkx3.2-ΔC gene mutant did not inhibit Pax7 expression (FIG. 4 panels C and D). Thus, the C-terminus of Nkx3.2 protein was required for Pax7 repression. Deletion of the C-terminus of Nkx3.2 did not completely abolish Nkx3.2-mediated MHC repression (FIG. 4 panels E and 4F). These data show that Nkx3.2 has an ability to inhibit MHC expression through additional domains other than the C-terminus. Replacement of the C-terminus with a VP16 activation domain was observed to significantly enhance expression of Pax3 and MHC, and the replacement did not lead to increased Pax7 expression in the satellite cells (See FIG. 4 panels B, D and F). Without being limited by any theory or particular mode of operation, it is envisioned that the effect of a fusion protein of Nkx3.2 having a deleted C-terminal domain replaced and a substituted VP16 transcriptional activation domain for the C-terminal domain (Nkx3.2ΔC-VP16) on Pax7 expression is a result of an intricate interaction of Pax7 with other myogenic factors, such as Pax3 and myogenin. Both Pax3 and myogenin inhibit Pax7 expression [42,43].

Immunochemical staining analysis showed that Nkx3.2 strongly inhibited MHC expression, and that the gene encoding a Nkx3.2 protein lacking the C-terminus domain inhibited MHC expression in muscle satellite cells. See FIG. 4 panel E. The Nkx3.2 gene mutant lacking the C-terminus domain inhibited MHC expression by about 50% as determined by relative MHC mRNA levels. FIG. 4 panel D. The Nkx3.2 with substituted VP16 transcriptional activation domain greatly enhanced muscle marker MHC expression as analyzed by immunochemistry (FIG. 4 panel E second row) and by relative MHC RNA levels compared to cells contacted with GFP (FIG. 1 panel D). The fusion protein of Nkx3.2 lacking the C-terminus domain and with a VP16 transcriptional activation domain resulted in a six-fold increase in the MHC RNA level compared to a Nkx3.2 mutant that lacks the C-terminus domain, and this vector Nkx3.2ΔC-VP16 resulted in 30-fold increase compared to MHC RNA levels of Nkx3.2 alone or GFP. FIG. 4 panel D. Thus, the C-terminal domain of Nkx3.2 plays an important role in the negative modulation (down regulation, repression) of muscle satellite cells to differentiate into mature muscle. A dominant negative gene mutant form of Nkx3.2 that lacks the C-terminal domain showed a reduced (at least five-fold less) inhibitory effect on MHC expression compared to a gene encoding wild type full length Nkx3.2 protein.

### Example 15

### Nkx3.2 Inhibits Pax3 Promoter Activity

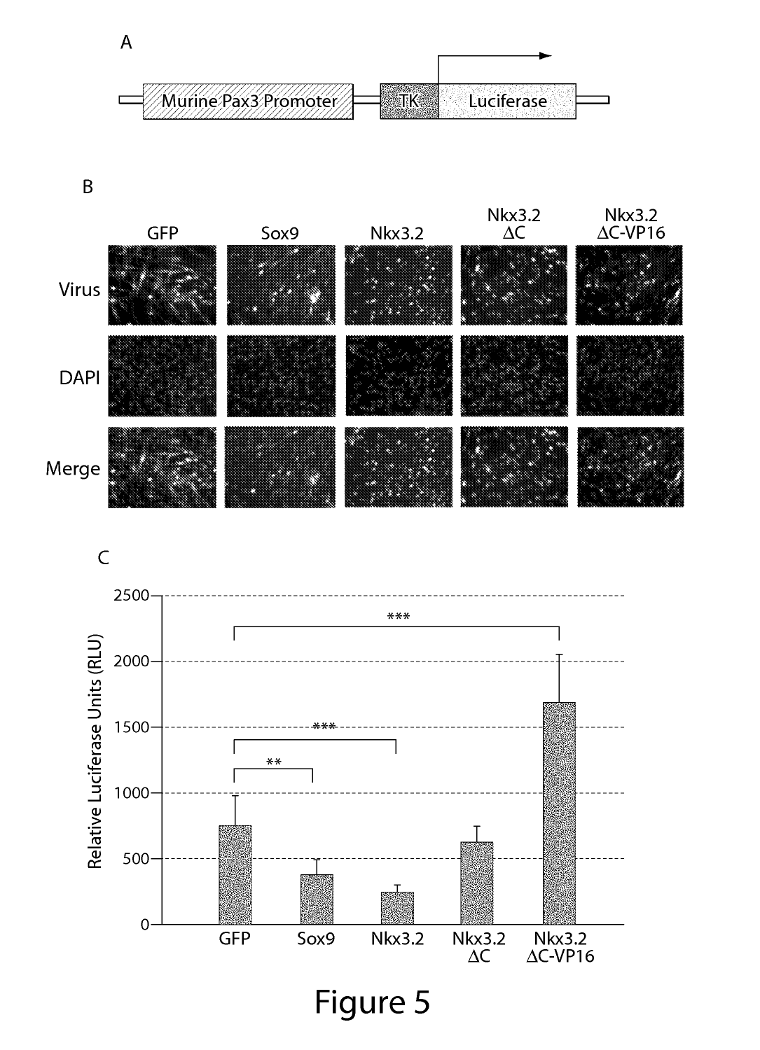

Nkx3.2 is shown in examples herein to act as a repressor to strongly inhibit Pax3 expression. A mouse Pax3 promoter sequence was previously identified from LacZ reporter analysis in transgenic mice that indicated that the mousePax3 promoter recapitulated endogenous Pax3 expression in the trunk [28]. A luciferase reporter was constructed to carry the murine Pax3 promoter sequence to and lucerifase assays showed that Nkx3.2 acted directly on the Pax3 promoter to inhibit its expression (FIG. 5 panel A). The effects of GFP and Sox9 on the Pax3 promoter were also evaluated herein.

Muscle satellite cells were contacted with retrovirus vectors that express Sox9V5, Nkx3.2-HA, Nkx3.2ΔC-HA, Nkx3.2ΔC-VP16, or control GFP. Efficiency of viral delivery using these methods was evaluated by immunohistochemistry (FIG. 5 panel B). Rhe Pax3 promoter luciferase construct was then transfected into the satellite cells. It was observed that muscle satellite cells contacted with Sox9 showed a moderate and significant reduction in Pax3 promoter activity. In contrast, muscle satellite cells contacted with a vector encoding Nkx3.2 showed increased Pax3 promoter inhibition activity (FIG. 5 panel C). A vector encoding a C-terminal deletion mutant of Nkx3.2 (Nkx3.2-ΔC) showed essentially no effect on the Pax3 promoter compared to control GFP-infected cells (FIG. 5 panel B).

The Nkx3.2 reverse function gene mutant (Nkx3.2-ΔC-VP16) activated the Pax3 promoter by at least two-fold (FIG. 5 panel C). These data show that Nkx3.2 and Sox9 inhibited muscle gene expression by inhibiting the Pax3 promoter.

### Example 16

### Promoting Cartilage Formation in Muscle Satellite Cell Using Vectors Encoding Nkx3.2 or Sox9

Differentiation of muscle satellite cells into chondrocytes involves repression of muscle cell fate and initiation of chondrogenesis. The roles of Nkx3.2 and Sox9 in the induction of cartilage genes in muscle satellite cells were herein examined. Cartilage expression was evaluated in cells contacted with vectors encoding Nkx3.2 or Sox9 constructs. The effects of Nkx3.2 or Sox9 vectors on expression of collagen II and aggrecan, markers associated with cartilage formation, was determined.

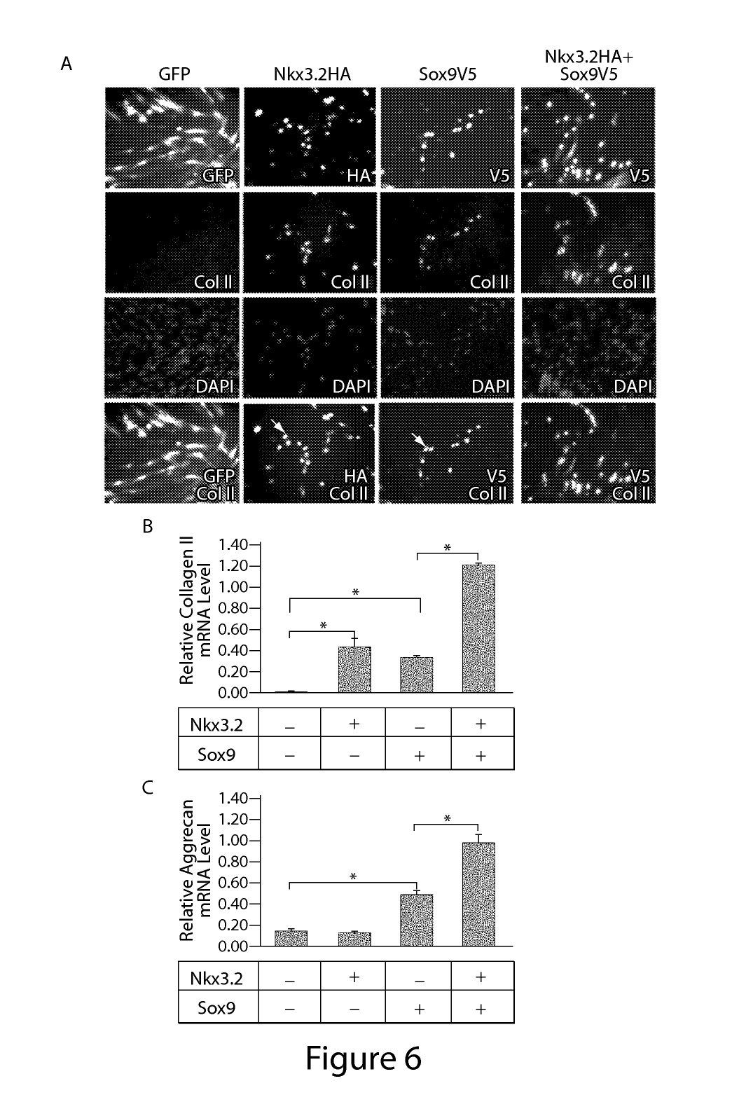

Intense collagen II staining in muscle satellite cells contacted with a vector encoding Nkx3.2 protein or a vector encoding Sox9 (FIG. 6 panel A) was observed, compared to control cells contacted with a vector encoding GFP. The amount of collagen II staining was greatest in cells contacted with both a vector encoding Nkx.3.2 and a vector encoding Sox9 (FIG. 6 panel A right column).

RT-PCR analysis was performed and data showed that each of Nkx3.2 and Sox9 induced muscle satellite cells to differentiate to cartilage. Presence of both proteins Nkx3.2 and Sox9 was observed to synergistically increase collagen II mRNA expression in muscle satellite cells by at least 150% (FIG. 6 panel B) and aggrecan by at least 100% (FIG. 6 panel C) compared to the RNA levels in presence of either Nkx3.2 or Sox9 alone.

A vector encoding Sox9 induced aggrecan expression in the muscle satellite cells, however contact with a vector encoding Nkx3.2 did not induce aggrecan expression (FIG. 6 panel C). Further, contacting muscle satellite cells with both a vector encoding Sox9 and a vector encoding Nkx3.2 resulted in a synergistic induction of aggrecan expression (FIG. 6 panel C).

Thus Nkx3.2 and Sox9 were observed to regulate expression of cartilage matrix components collagen II and aggrecan differently. Specifically, Nkx3.2 and Sox9 both induced collagen II expression in muscle satellite cells, and a synergistic effect was observed for aggrecan expression for cells contacted with both transcription factors. These data show that the interaction of Nkx3.2 and Sox9 plays an important role in promotion of chondrogenic differentiation in muscle satellite cells.

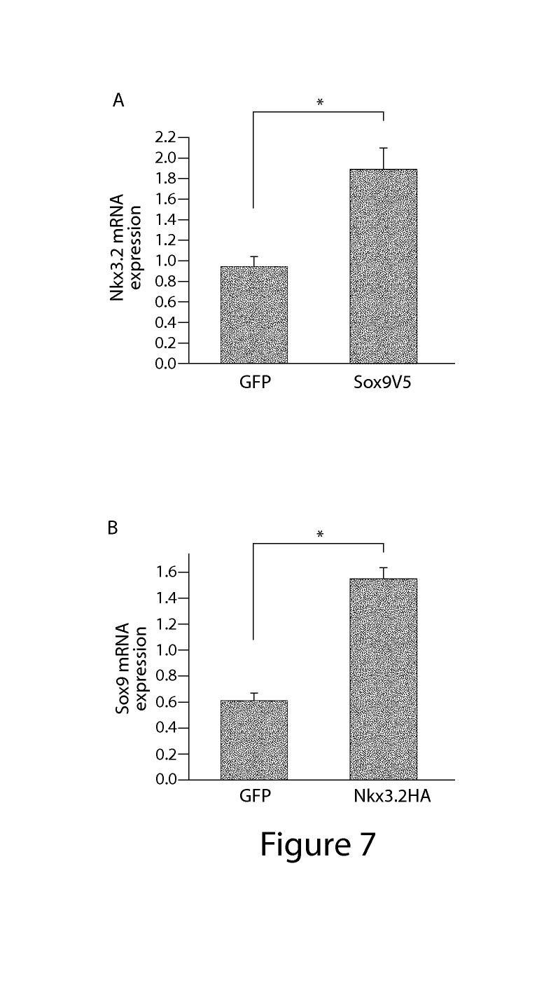

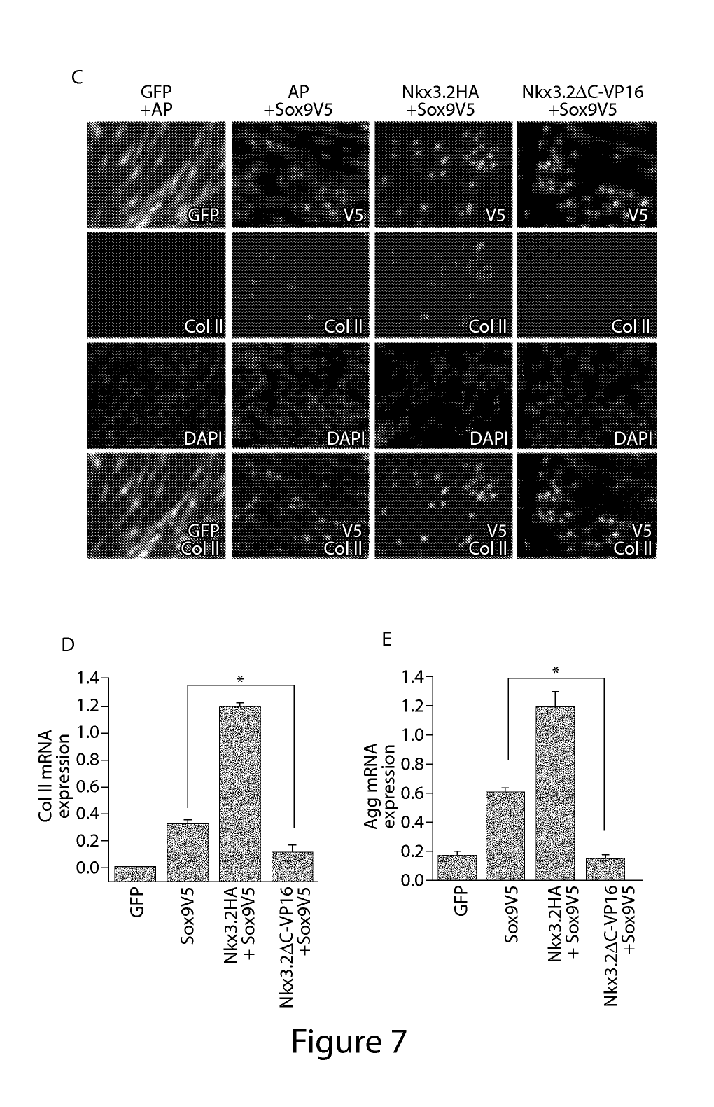

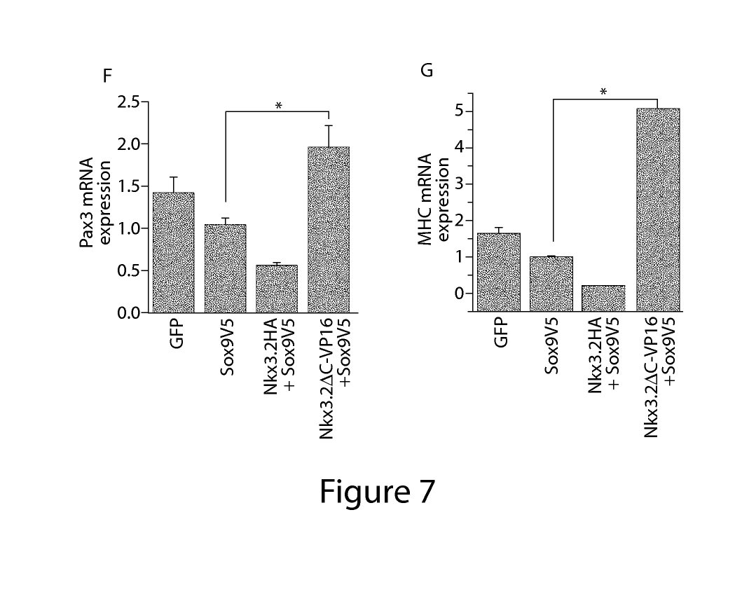

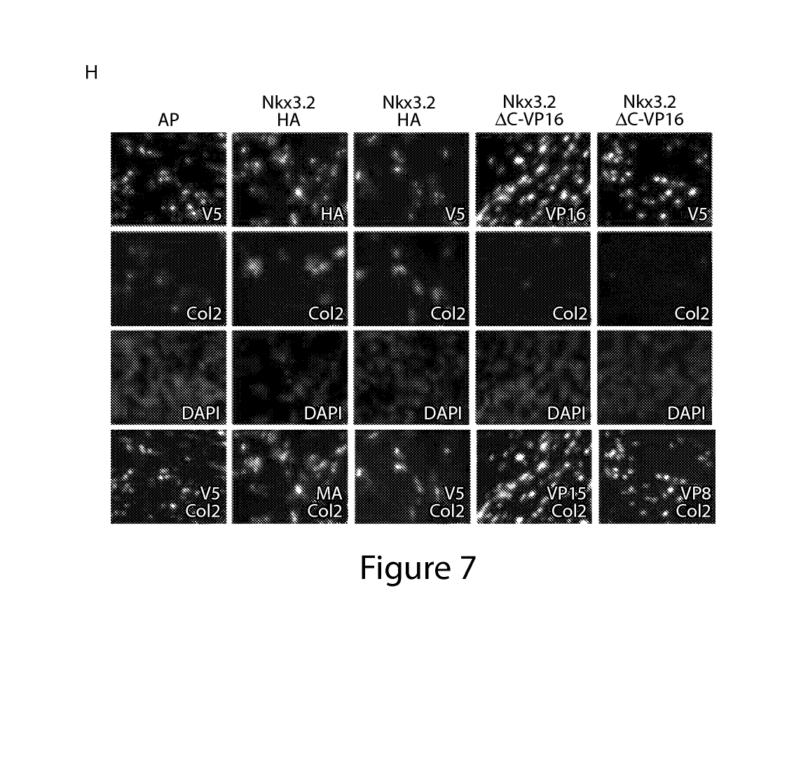

Nkx3.2 induced greater mRNA expression of Sox 9, and Sox9 contacted cells induced greater mRNA expression of Nkx3.2 (compare FIG. 7 panel A with FIG. 7 panel B). Thus, Nkx3.2 and Sox9 transcription factors were observed to form a positive regulatory loop (FIG. 7 panels A and B), as increased expression of transcription factor Nkx3.2 increased expression of transcription factor Sox9, which in turn induced increased expression of Nkx3.2.

Cells were analyzed by qRT-PCT for mRNA expression of each of collagen II (FIG. 7 panel D) and aggrecan (FIG. 7 panel E). Nkx3.2 was observed to be required for Sox9 to induce cartilage formation, as the reverse-function mutant of an Nkx3.2 that lacks the C-terminus domain and has a VP16 transcription domain attached to the C-terminal domain end of Nkx3.2 strongly blocked the ability of Sox9 to turn on the cartilage program. See FIG. 7 panel D.

Data from qRT-PCT show that blocking Nkx3.2 expression using a reverse function Nkx3.2 gene mutant prevented Sox9 from inducing expression of both collagen II and aggrecan (FIG. 7 panel D and E). FIG. 7 panel G shows immunochemical data for collagen II protein which was visible in cells contacted with vectors expressing Nkx3.2. Nkx3.2 contacted-cells were induced to form cartilage and bone. Data show little or no collagen II expressed in cells contacted with the reverse function Nkx3.2 mutant that lacks the C-terminus domain and has a VP16 transcription domain attached to the C-terminal domain end of Nkx3.2, and that these cells were strongly induced to differentiate to mature muscle and expressed little collagen II protein. FIG. 7 panel H second row. Strong MHC staining was observed in cells contacted with the Nkx3.2 mutant that lacks the C-terminus domain and has a VP16 transcription domain was observed. FIG. 4 panel F.

### Example 17

### A Reverse Function Mutant of Nkx3.2 Inhibited Ability of Sox9 to Induce Chondrogenesis and Inhibited Myogenesis in Muscle Satellite Cells

Nkx3.2 and Sox9 were observed herein to promote chondrogenic differentiation of muscle satellite cells. The relationship between these factors transcription factors in chondrogenesis and myogenesis were investigated, and data showed that Nkx3.2 and Sox9 induced expression of the other in satellite cells (FIG. 7 panels A and B). Sox9 strongly induced chondrogenesis as shown herein, this induction may be due to direct activation of promoters of cartilage matrix genes [36,44,45]. Data herein show further that Sox9 has a weak activity in inhibiting myogenesis. Nkx3.2 exerts a stronger inhibitory activity on Pax3, the myogenic factor that inhibits chondrogenesis.

To determine whether the activity of Sox9 on chondrogenesis and myogenesis in satellite cells is attributed to induction of Nkx3.2, examples used of a reverse function mutant of Nkx3.2 (Nkx3.2-ΔC-VP16), to evaluate whether this gene mutant, acting in a dominant-negative manner, inhibited the ability of Sox9 to induce chondrogenesis.

Surprisingly, data herein show that although muscle satellite cells contacted with a vector encoding Sox9 dramatically upregulated collagen II expression, muscle satellite cells contacted both with a vector encoding Nkx3.2ΔC-VP16 and a vector encoding Sox9 resulted in a dramatic reduction in this cartilage matrix protein (FIG. 7 panel C). RT-PCR analysis showed diminished expression of collagen II mRNA as well as aggrecan mRNA for muscle satellite cells contacted with both a vector encoding Nkx3.2ΔC-VP16 and a vector encoding Sox9 (FIG. 7 panels D and E).

To determine whether Nkx3.2 is required for the weak inhibitory activity of Sox9 on muscle gene expression, muscle satellite cells were contacted with both a vector encoding Nkx3.2ΔC-VP16 and a vector encoding Sox9 and analyzed for Pax3 and MHC expression. Data showed that contacting muscle satellite cells with vectors encoding Nkx3.2ΔC-VP16 and Sox9 completely abolished the ability of Sox9 to inhibit Pax3 and MHC expression (FIG. 7 panels F and G). Sox9 alone was unable to induce chondrogenesis in muscle satellite cells in the absence of the activity of Nkx3.2. Clearly Sox9 inhibited the myogenic program in the satellite cells through the induction of Nkx3.2 expression.

### Example 18

### Modulating Muscle Satellite Cells Using Modified Nkx3.2 Constructs

To determine the effect of Nkx3.2 and Sox9 to modulate trans-differentiation of muscle satellite cells, modifications i.e., deletions and substitutions of amino acids in both the C-terminal domain and N-terminal domain are engineered into genes encoding the amino acid sequences of human Nkx3.2 and Sox9.

The modified human proteins are expressed by mammalian and/or vertebrate vectors. Tissue with symptoms of heterotopic ossification including cartilage-like and bone-like masses in the soft tissue is contacted in vivo with vectors expressing one of the naturally-occurring or modified transcription factor constructs to compare these proteins as potential improved therapeutic agents, and to determine whether these constructs are more efficient for treating subjects than agents bisphosphonates or radiation therapy.

Analyses are performed to determine the extent that vectors expressing modified human Nkx3.2 protein and/or Sox9 proteins protect tissues and cells from trans-differentiation into cartilage and bone masses associated with heterotopic ossification. The vectors expressing modified human Nkx3.2 proteins and Sox9 proteins are tested also in an ex vivo model.

Results are predicted to indicate that these constructs are modulators of trans-differentiation and are potential therapeutic agents for heterotopic ossification.

### Example 19

### Induction of Nkx3.2 and Sox9 in the Muscle Progenitor Cells Contributes to Cartilage Formation and Fracture Repair in an In Vivo Mouse Model of Bone Fracture Healing

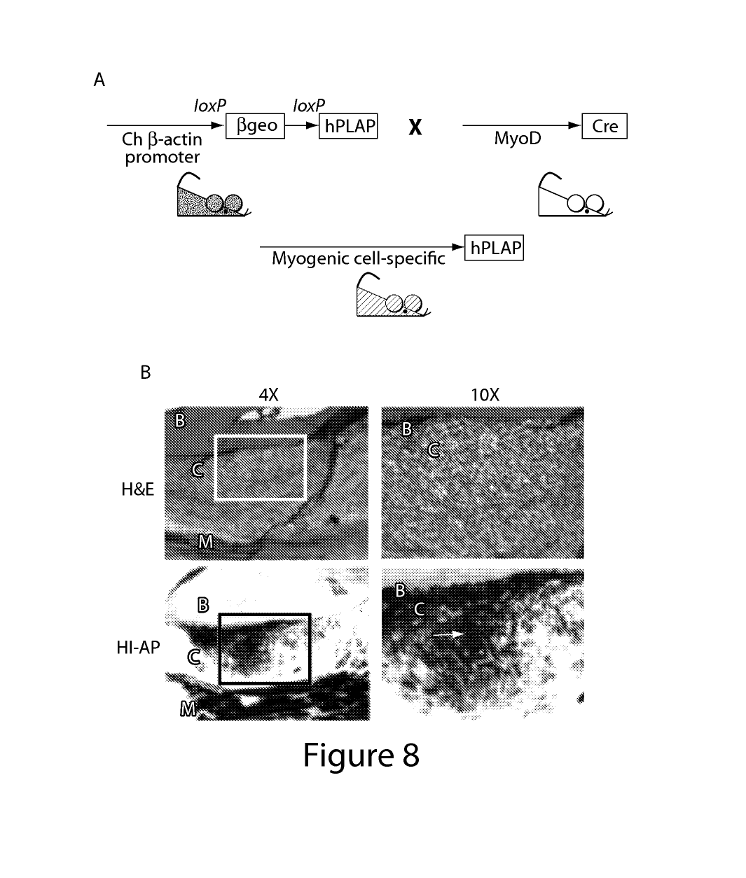

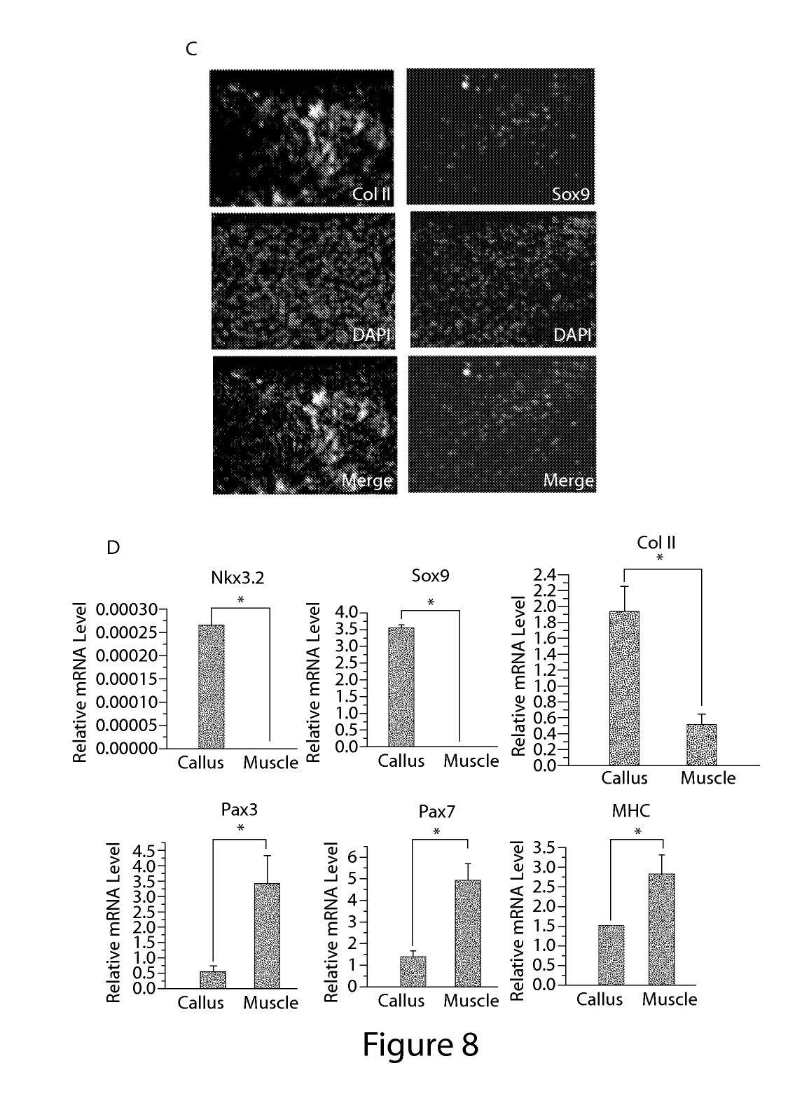

To establish the in vivo significance of Nkx3.2 and Sox9 in the chondrogenic differentiation of muscle satellite cells, a bone fracture healing model was prepared, and the expression of these transcription factors in the myogenic progenitor cells that give rise to chondrocytes during fracture healing was analyzed. A MyoD-cre:Z/AP mouse was generated by crossing two transgenic lines. See FIG. 8 panel A and Example 8 herein. The MyoD-driven Cre mouse line allows Cre to be expressed in throughout muscle progenitor cells, including satellite cells [4].

It was observed that upon Cre-mediated recombination, the Z/AP line permanently expressed the human placental alkaline phosphatase (hPLAP) reporter gene in affected cells (FIG. 8 panel A). The heat-stable property of hPLAP allowed for a clear identification of the expression of this reporter compared to expression of endogenous alkaline phosphatase, which is abundantly expressed in bone cells [9]. Therefore, in this MyoD-Cre+:Z/AP+ mouse, satellite cells and their progenitors expressed heat-stable alkaline phosphatase, which was marked by a characteristic purple stain using enzymatic reactions (FIG. 8 panel A).

The MyoD-Cre+:Z/AP+ mouse was subjected to open tibial midshaft fractures, and abundant amounts of muscle progenitor cells and progenitor cell descendants were visualized in the fracture callus region (FIG. 8 panel B). Muscle progenitor cells undergo cartilage and bone differentiation during the process of fracture healing [46,47]. Data show that in addition to contributing to the fracture callus, the hPLAP-(+) cells herein marked the muscle next to the bone, and not the endogenous osteocytes within the bone (FIG. 8 panel B). Clearly the methods herein using alkaline phosphatase and Cre-mediated recombination were effective for producing the bone fracture healing model.

Immunohistochemistry (IHC) analysis was performed on sections of the fracture region to evaluate whether Sox9 and Nkx3.2 were induced in the muscle progenitor cells (FIG. 8 panel C). Data showed that Sox9 was strongly expressed in the cells in the fracture callus, an area of the bone which correlated with the site of induction of cartilage marker collagen II expression (FIG. 8 panel C). Laser capture microscopy (LCM) was performed to evaluate Nkx3.2 expression in the fractures and to determine whether Nkx3.2 and Sox9 are indeed induced in the muscle progenitor cells that give rise to chondrocytes in the fracture healing process. Muscle progenitor cells were isolated by LCM and identified with alkaline phosphatase (HI-AP) staining. A comparison was made between expression of muscle and cartilage markers in the fracture callus to expression patterns in neighboring muscle cells (FIG. 8 panel D). Data showed increased expression Nkx3.2, Sox9, and collagen II in muscle progenitor cells in the fracture callus compared to progenitor cells in the muscle (FIG. 8 panel D). It was also observed that in progenitor cells, expression of muscle markers Pax3, Pax7 and myosin heavy chain was downregulated compared to progenitors cells in the neighboring muscle regions (FIG. 8 panel D). These data show that Nkx3.2 and Sox9 were expressed in the muscle progenitor cells that contribute to cartilage formation during fracture healing, and that these transcription factors promote chondrogenic differentiation of satellite cells during fracture healing.

### REFERENCES

1. Buckingham M, Bajard L, Chang T, Daubas P, Hadchouel J, et al. (2003) The formation of skeletal muscle: from somite to limb. J Anat 202: 59-68.
2. Kuang S, Kuroda K, Le Grand F, Rudnicki M A (2007) Asymmetric self-renewal and commitment of satellite stem cells in muscle. Cell 129: 999-1010.
3. Relaix F, Rocancourt D, Mansouri A, Buckingham M (2005) A Pax3/Pax7-dependent population of skeletal muscle progenitor cells. Nature 435: 948-953.
4. Kanisicak O, Mendez J J, Yamamoto S, Yamamoto M, Goldhamer D J (2009) Progenitors of skeletal muscle satellite cells express the muscle determination gene, MyoD. Dev Biol 332: 131-141.
5. Bryson-Richardson R J, Currie P D (2008) The genetics of vertebrate myogenesis. Nat Rev Genet 9: 632-646.
6. Shefer G, Wleklinski-Lee M. Yablonka-Reuveni Z (2004) Skeletal muscle satellite cells can spontaneously enter an alternative mesenchymal pathway. J Cell Sci 117: 5393-5404.
7. Morrison J I, Loof S, He P, Simon A (2006) Salamander limb regeneration involves the activation of a multipotent skeletal muscle satellite cell population. J Cell Biol 172: 433-440.
8. Wada M R, Inagawa-Ogashiwa M, Shimizu S, Yasumoto S, Hashimoto N (2002) Generation of different fates from multipotent muscle stem cells. Development 129: 2987-2995.
9. Hashimoto N, Kiyono T, Wada M R, Umeda R, Goto Y, et al. (2008) Osteogenic properties of human myogenic progenitor cells. Mech Dev 125: 257-269.
10. Katagiri T, Yamaguchi A, Komaki M, Abe E, Takahashi N, et al. (1994) Bone morphogenetic protein-2 converts the differentiation pathway of C2C12 myoblasts into the osteoblast lineage. J Cell Biol 127: 1755-1766.
11. Morrison J I, Borg P, Simon A (2010) Plasticity and recovery of skeletal muscle satellite cells during limb regeneration. FASEB J 24: 750-756.
12. Liu R, Schindeler A, Little D G (2010) The potential role of muscle in bone repair. J Musculoskelet Neuronal Interact 10: 71-76.
13. Liu r, Birke O, Morse A, Peacock L, Mikulec K, et al. (2011) Myogenic progenitors contribute to open but not closed fracture repair. BMC Musculoskelet Disord.
14. Schindeler A, Little D G (2008) Recent insights into bone development, homeostasis, and repair in type 1 neurofibromatosis (NF1). Bone 42: 616-622.
15. Schindeler A, McDonald M M, Bokko P, Little D G (2008) Bone remodeling during fracture repair: The cellular picture. Semin Cell Dev Biol 19: 459-466.
16. Harry L E, Sandison A, Paleolog E M, Hansen U, Pearse M F, et al. (2008) Comparison of the healing of open tibial fractures covered with either muscle or fasciocutaneous tissue in a murine model. J Orthop Res 26: 1238-1244.
17. Evans C H, Liu F J, Glatt V, Hoyland I A, Kirker-Head C, et al. (2009) Use of genetically modified muscle and fat grafts to repair defects in bone and cartilage. Eur Cell Mater 18: 96-111.
18. Bettex-Galland M, Wiesmann U (1987) Differentiation of L6 myoblastic cells into chondrocytes. Experientia 43: 610-611.
19. Li G, Peng H, Corsi K, Usa A, Gearhart B, et al. (2004) BMP4 can induce C2C12 myoblasts to differentiate into chondrocytes. Proceedings 50th annual meeting of the orthopaedic research society: poster number: 0912.
20. Nathanson M A (1983) Proteoglycan synthesis by skeletal muscle undergoing bone matrix-directed transformation into cartilage in vitro. J Biol Chem 258: 10325-10334.
21. Nathanson M A (1986) Transdifferentiation of skeletal muscle into cartilage: transformation or differentiation? Curr Top Dev Biol 20: 39-62.
22. Burton N M, Vierck J, Krabbenhoft L, Bryne K, Dodson M V (2000) Methods for animal satellite cell culture under a variety of conditions. Methods Cell Sci 22: 51-61.
23. Sekiya I, Larson B L, Vuoristo J T, Reger R L, Prockop D J (2005) Comparison of effect of BMP-2, -4, and -6 on in vitro cartilage formation of human adult stem cells from hone marrow stroma. Cell Tissue Res 320: 269-276.
24. Mello M A, Tuan R S (1999) High density micromass cultures of embryonic limb bud mesenchymal cells: an in vitro model of endochondral skeletal development. In Vitro Cell Dev Biol Anim 35: 262-269.
25. Cairns D M, Sato M E, Lee P G, Lassar A B, Zeng L (2008) A gradient of Shh establishes mutually repressing somitic cell fates induced by Nkx3.2 and Pax3. Dev Biol 323: 152-165.
26. Zeng L, Kempf H, Murtaugh L C, Sato M E, Lassar A B (2002) Shh establishes an Nkx3.2/Sox9 autoregulatory loop that is maintained by BMP signals to induce somitic chondrogenesis. Genes Dev 16: 1990-2005.
27. Provot S, Kempf H, Murtaugh L C, Chung U I, Kim D W, et al. (2006) Nkx3.2/Bapx1 acts as a negative regulator of chondrocyte maturation. Development 133: 651-662.
28, Natoli T A, Ellsworth M K, Wu C, Gross K W, Pruitt S C (1997) Positive and negative DNA sequence elements are required to establish the pattern of Pax3 expression. Development 124: 617-626.
29. Espina V, Milia J, Wu G, Cowherd S, Liotta L A (2006) Laser capture microdissection. Methods Mol Biol 319: 213-229.
30. Espina V, Wulfkuhle J D, Calvert V S, VanMeter A, Zhou W, et al. (2006) Laser-capture microdissection. Nat Protoc 1: 586-603.
31. Lobe C G, Koop K E, Kreppner W, Lomeli H, Gertsenstein M, et al. (1999) Z/AP, a double reporter for cre-mediated recombination. Dev Biol 208: 281-292.
32. Schindeler A, Morse A, Harry L, Godfrey C, Mikulec K, et al. (2008) Models of tibial fracture healing in normal and Nf1-deficient mice. J Orthop Res 26: 1053-1060.
33. Hartley R S, Bandman E, Yablonka-Reuveni Z (1992) Skeletal muscle satellite cells appear during late chicken embryogenesis. Dev Biol 153: 206-216.
34. Evans C H, Palmer G D, Pascher A, Porter R, Kwong F N, et al. (2007) Facilitated endogenous repair: making tissue engineering simple, practical, and economical. Tissue Eng 13: 1987-1993.
35. Akiyama H, Chaboissier M C, Martin J F, Schedl A, De Crombrugghe B (2002) The transcription factor Sox9 has essential roles in successive steps of the chondrocyte differentiation pathway and is required for expression of Sox5 and Sox6. Genes Dev 16: 2813-2828.
36. Bell D M, Leung K K, Wheatley S C, Ng L J, Zhou S, et al. (1997) SOX9 directly regulates the type-II collagen gene. Nat Genet 16: 174-178.
37. Bi W, Deng J M, Zhang Z, Behringer R R, de Crombrugghe B (1999) Sox9 is required for cartilage formation. Nat Genet 22: 85-89.
38. Lettice L A, Purdie L A, Carlson G J, Kilanowski F, Dorin J, et al. (1999) The mouse bagpipe gene controls development of axial skeleton, skull, and spleen. Proc Natl Acad Sci USA 96: 9695-9700.
39. Tribioli C, Lufkin T (1999) The murine Bapx1 homeobox gene plays a critical role in embryonic development of the axial skeleton and spleen. Development 126: 5699-5711.
40. Murtaugh L C, Zeng L, Chyung J H, Lassar A B (2001) The chick transcriptional repressor Nkx3.2 acts downstream of Shh to promote BMP-dependent axial chondrogenesis. Developmental Cell 1: 411-422.
41. Kim D W, Lassar A B (2003) Smad-dependent recruitment of a histone deacetylase/Sin3A complex modulates the bone morphogenetic protein-dependent transcriptional repressor activity of Nkx3.2. Mol Cell Biol 23: 8704-8717.
42. Borycki A G, Li J, Jin F, Emerson C P, Epstein J A (1999) Pax3 functions in cell survival and in pax7 regulation. Development 126: 1665-1674.
43. Olguin H C, Yang Z, Tapscott S J, Olwin B B (2007) Reciprocal inhibition between Pax7 and muscle regulatory factors modulates myogenic cell fate determination. J Cell Biol 177: 769-779.
44. Lefebvre V, Huang W, Harley V R, Goodfellow P N, de Crombrugghe B (1997) SOX9 is a potent activator of the chondrocyte-specific enhancer of the pro alpha1(II) collagen gene. Mol Cell Biol 17: 2336-2346.
45. Sekiya I, Tsuji K, Koopman P, Watanabe H, Yamada Y, et al. (2000) SOX9 enhances aggrecan gene promoter/enhancer activity and is up-regulated by retinoic acid in a cartilage-derived cell line, TC6. J Biol Chem 275: 10738-10744.
46. Rosier R N, O'Keefe R J, Hicks D G (1998) The potential role of transforming growth factor beta in fracture healing. Clin Orthop Relat Res: S294-300.
47. Xie C, Liang B, Xue M, Lin A S, Loiselle A, et al. (2009) Rescue of impaired fracture healing in COX-2−/− mice via activation of prostaglandin E2 receptor subtype 4. Am J Pathol 175: 772-785.
48. Joe A W, Yi L, Natarajan A, Le Grand F, So L, et al. (2010) Muscle injury activates resident fibro/adipogenic progenitors that facilitate myogenesis. Nat Cell Biol 12: 153-163.
49. Uezumi A, Fukada S, Yamamoto N, Takeda S, Tsuchida K (2010) Mesenchymal progenitors distinct from satellite cells contribute to ectopic fat cell formation in skeletal muscle. Nat Cell Biol 12: 143-152.
50. Bosch P, Musgrave D S, Lee J Y, Cummins J, Shuler T, et al. (2000) Osteoprogenitor cells within skeletal muscle. J Orthop Res 18: 933-944.
51. Lee J Y, Qu-Petersen Z, Cao B, Kimura S, Jankowski R, et al. (2000) Clonal isolation of muscle-derived cells capable of enhancing muscle regeneration and bone healing. J Cell Biol 150: 1085-1100.
52. Starkey J D, Yamamoto M, Yamamoto S, Goldhamer D J (2011) Skeletal muscle satellite cells are committed to myogenesis and do not spontaneously adopt nonmyogenic fates. J Histochem Cytochem 59: 33-46.
53. Meech R, Gomez M, Woolley C, Barro M, Hulin J A, et al. (2010) The homeobox transcription factor Barx2 regulates plasticity of young primary myofibers. PLoS ONE 5: e11612.
54. Odelberg S J, Kollhoff A, Keating M T (2000) Dedifferentiation of mammalian myotubes induced by msx1. Cell 103: 1099-1109.
55. Schindeler A, Liu R, Little D G (2009) The contribution of different cell lineages to bone repair: exploring a role for muscle stem cells. Differentiation 77: 12-18.
56. Colnot C (2009) Skeletal cell fate decisions within periosteum and bone marrow during bone regeneration. J Bone Miner Res 24: 274-282.
57. Doherty M J, Ashton B A, Walsh S, Beresford J N, Grant M E, et al. (1998) Vascular pericytes express osteogenic potential in vitro and in vivo. J Bone Miner Res 13: 828-838.
58. Medici D, Shore E M, Lounev V Y, Kaplan F S, Kalluri R, et al. (2010) Conversion of vascular endothelial cells into multipotent stem-like cells. Nat Med 16: 1400-1406.
59. Lu C. Marcucio R, Miclau T (2006) Assessing angiogenesis during fracture healing. Iowa Orthop J 26: 17-26.
60. Bandyopadhyay A, Tsuji K, Cox K, Harfe B D, Rosen V, et al. (2006) Genetic analysis of the roles of BMP2, BMP4, and BMP7 in limb patterning and skeletogenesis. PLoS Genet 2: e216.
61. Clark C A, Li T F, Kim K O, Drissi H, Zuscik M J, et al. (2009) Prostaglandin E2 inhibits BMP signaling and delays chondrocyte maturation. J Orthop Res 27: 785-792.
62. Yu Y Y, Lieu S, Lu C, Miclau T, Marcucio R S, et al. (2010) Immunolocalization of BMPs, BMP antagonists, receptors, and effectors during fracture repair. Bone 46: 841-851.
63. Yi S E, Daluiski A, Pederson R, Rosen V, Lyons K M (2000) The type I BMP receptor BMPRIB is required for chondrogenesis in the mouse limb. Development 127: 621-630.
64. Minear S, Leucht P, Jiang J, Liu B, Zeng A, et al. (2010) Wnt proteins promote bone regeneration. Sci Transl Med 2: 29ra30.
65. Minear S, Leucht P, Miller S, Helms J A (2010) rBMP represses Wnt signaling and influences skeletal progenitor cell fate specification during bone repair. J Bone Miner Res 25: 1196-1207.
66. Secreto F J, Hoeppner L H, Westendorf J J (2009) Wnt signaling during fracture repair. Curr Osteoporos Rep 7: 64-69.
67. Kaback L A, Soung do Y, Naik A, Geneau G, Schwarz E M, et al. (2008) Teriparatide (1-34 human PTH) regulation of osterix during fracture repair. J Cell Biochem 105: 219-226.
68. Lissenberg-Thunnissen S N, de Gorter D J, Sier C F, Schipper 113 (2011) Use and efficacy of bone morphogenetic proteins in fracture healing. Int Orthop.
69. Takahata M, Awad H A, O'Keefe R J, Bukata S V, Schwarz E M (2011) Endogenous tissue engineering: PTH therapy for skeletal repair. Cell Tissue Res.
70. Buckingham M, Relaix F (2007) The role of Pax genes in the development of tissues and organs: Pax3 and Pax7 regulate muscle progenitor cell functions. Annu Rev Cell Dev Biol 23: 645-673.
71. Relaix F. Montarras D, Zaffran S, Gayraud-Morel B, Rocancourt D, et al. (2006) Pax3 and Pax7 have distinct and overlapping functions in adult muscle progenitor cells. J Cell Biol 172: 91-102.
72. Maroto M. Reshef R, Munsterberg A E, Koester S, Goulding M, et al. (1997) Ectopic Pax-3 activates MyoD and Myf-5 expression in embryonic mesoderm and neural tissue. Cell 89: 139-148.
73. Bajard L, Relaix F, Lagha M, Rocancourt D, Daubas P, et al. (2006) A novel genetic hierarchy functions during hypaxial myogenesis: Pax3 directly activates Myf5 in muscle progenitor cells in the limb. Genes Dev 20: 2450-2464.
74. Chalepakis G, Jones F S, Edelman G M, Gruss P (1994) Pax-3 contains domains for transcription activation and transcription inhibition. Proc Natl Acad Sci USA 91: 12745-12749.
75. Epstein J A, Lam P, Jepeal L, Maas R L, Shapiro D N (1995) Pax3 inhibits myogenic differentiation of cultured myoblast cells. J Biol Chem 270: 11719-11722.
76. Hollenbach A D, Sublett J E, McPherson C J, Grosveld G (1999) The Pax3-FKHR oncoprotein is unresponsive to the Pax3-associated repressor hDaxx. EMBO J 18: 3702-3711.
77. Magnaghi P, Roberts C, Lorain S, Lipinski M, Scambler P J (1998) HIRA, a mammalian homologue of Saccharomyces cerevisiae transcriptional co-repressors, interacts with Pax3. Nat Genet 20: 74-77.
78. Wiggan O, Taniguchi-Sidle A, Hamel P A (1998) Interaction of the pRB-family proteins with factors containing paired-like homeodomains. Oncogene 16: 227-236.
79. Hernandez-Hernandez J M, Delgado-Olguin P, Aguillon-Huerta V, Furlan-Magaril M, Recillas-Targa F, et al. (2009) Sox9 represses alpha-sarcoglycan gene expression in early myogenic differentiation. J Mol Biol 394: 1-14.
80. Schmidt K, Glaser G, Wernig A, Wegner M, Rosorius O (2003) Sox8 is a specific marker for muscle satellite cells and inhibits myogenesis. J Biol Chem 278: 29769-29775.
81. Wright E, Hargrave M R, Christiansen J, Cooper I., Kun J, et al. (1995) The Sry-related gene Sox9 is expressed during chondrogenesis in mouse embryos. Nat Genet 9: 15-20.
82. Hattori T, Muller C, Gebhard S, Bauer E, Pausch F, et al. (2010) SOX9 is a major negative regulator of cartilage vascularization, bone marrow formation and endochondral ossification. Development 137: 901-911.
83. Herbrand H, Pabst O, Hill R, Arnold H-H (2002) Transcription factors Nkx3.1 and Nkx3.2 (Bapx1) play an overlapping role in sclerotome development of the mouse. Mech of Dev 117: 217-224.
84. Lettice L, Hecksher-Sorensen J, Hill R (2001) The role of Bapx1 (Nkx3.2) in the development and evolution of the axial skeleton. J Anat 199: 181-187.
85. Hellemans J, Simon M, Dheedene A, Alanay Y, Mihci E, et al. (2009) Homozygous inactivating mutations in the NKX3-2 gene result in spondylo-megaepiphyseal-metaphyseal dysplasia. Am J Hum Genet 85: 916-922.
86. Lengner C J, Hassan M Q, Serra R W, Lepper C, van Wijnen A J, et al. (2005) Nkx3.2-mediated repression of Runx2 promotes chondrogenic differentiation. J Biol Chem 280: 15872-15879.

What is claimed is:

1. A pharmaceutical composition for modulating trans-differentiation of muscle satellite cells, the pharmaceutical composition comprising:
a modulator of trans-differentiation of the muscle satellite cells selected from the group of:
a transcription factor
a nucleic acid sequence encoding expression of the transcription factor, and
an agent that binds to the transcription factor, wherein the transcription factor is selected from the group consisting of a homeodomain class transcription factor and a TATA binding protein class transcription factor and comprises
wherein the modulator has at least one nucleotide binding-domain,
wherein the transcription factor modulates trans-differentiation of the muscle satellite cells to chondrocytes and bone.

2. The composition according to claim 1, wherein the transcription factor comprises an NKX protein or a portion thereof or a Sox protein or a portion thereof.

3-5. (canceled)

6. The composition according to claim 1, wherein the composition comprises a fusion protein of the transcription factor.

7. The composition according to claim 1, wherein the transcription factor alleviates a symptom of a disease or a disorder, for example the disease or disorder is selected from the group of: heterotopic ossification; edema; formation of a tissue mass; joint or muscle stiffness; joint or muscle pain; and arthritis.

8. The composition according to claim 1, wherein the transcription factor or the agent improves fracture healing.

9. The composition according to claim 1, wherein the agent that binds the transcription factor comprises a transcription repressor or a siRNA that negatively modulates a nucleic acid that encodes the transcription factor.

10. (canceled)

11. The composition according to claim 1 effective for increasing formation of cartilage or bone in a subject, for example increasing formation in the subject having a deficiency, defect, or fracture of the cartilage or the bone, wherein the pharmaceutical composition is optionally compound to be administered by at least one technique selected from the group consisting of topically, ocularly, nasally, bucally, orally, rectally, parenterally, intracistemally, intravaginally, and intraperitoneally.

12. A method for modulating trans-differentiation of muscle satellite cells of a subject, the method comprising:
engineering a modulator of trans-differentiation of the muscle satellite cells, wherein the modulator is selected from the group of: a transcription factor, a nucleic acid sequence encoding expression of the transcription factor, and an agent that binds to the transcription factor,
wherein the transcription factor is selected from a homeodomain class transcription factor and a TATA binding protein class transcription factor, and comprises at least one nucleotide binding-domain;
contacting cells or a tissue with the modulator; and,
measuring an amount of at least one phenotype selected from chondrocyte, muscle, and bone, in comparison to cells or tissue not so contacted and otherwise identical, wherein an increase or a decrease in the phenotype in the cells or the tissue is an indication of modulation of the muscle satellite cells.

13. The method according to claim 12, wherein engineering the modulator comprises expressing in the cells or the tissue a gene encoding an NKX protein or a portion thereof, a Sox protein or a portion thereof, and a siRNA that targets a nucleic acid having a sequence encoding the transcription factor or encoding the agent that binds to the transcription factor.

14-17. (canceled)

18. The method according to claim 12, wherein engineering the modulator comprises constructing a nucleic acid vector carrying the gene encoding the transcription factor; or a viral vector carrying a gene encoding the transcription factor.

19. The method according to claim 12, wherein engineering the modulator comprises expressing a fusion protein in the cells or the tissue for example a fusion protein comprising Nkx3.2 and a VP16 transcriptional domain.

20-23. (canceled)

24. The method according to claim 12, wherein contacting the cells or the tissue with the modulator further comprises contacting with at least one selected from the group of: a coactivator, a transcription repressor, a transcription enhancer, and a growth factor.

25. (canceled)

26. The method according to claim 12, wherein measuring the amount of the at least one phenotype comprises measuring at least one from the group of: myosin for example myosin heavy chain or myosin light chain; an actin; an actin/myosin complex; a collagen; hyaluronan; aggrecan; a paired box protein for example Pax3 or Pax 7; alkaline phosphatase; osteocalcin; and procollagen type 1 N-terminal propeptide.

27. The method according to claim 12, wherein after measuring the amount of the at least one phenotype, the method further comprises observing at least one selected from the group of: remediation of a disease or condition for example heterotopic ossification; edema; formation of a mass of tissue comprising cartilaginous material or bone material; decreased joint or muscle stiffness; decreased joint or muscle pain; remediation of arthritis; improved or increased bone fracture healing, and improved healing in the tibia, fibula, or femur.

28-34. (canceled)

35. A kit for modulating the trans-differentiation of muscle satellite cells, the kit comprising:
a modulator of trans-differentiation of the muscle satellite cells, wherein the modulator is selected from the group of: a transcription factor, a nucleic acid sequence encoding expression of the transcription factor, and an agent that binds to the transcription factor,
wherein the transcription factor is selected from a homeodomain class transcription factor such as Nkx3.2 and a TATA binding protein class transcription factor such as Sox9, and comprises at least one nucleotide binding-domain;
a container; and,
instructions for use.

36. (canceled)

37. The kit according to claim 35, wherein the agent that binds to the transcription factor comprises a repressor that binds to Nkx3.2 or Sox9.

38. (canceled)

39. The kit according to 35, wherein the transcription factor comprises an at least one of the group consisting of NKX protein or a portion thereof or a Sox protein or a portion thereof.

40-66. (canceled)

67. The composition according to claim 1, wherein the composition is compounded as a medicament for promoting trans-differentiation of at least one cell or tissue.

68. The composition according to claim 67, wherein the at least one cell or the tissue comprises cartilage, muscle, or bone.

69. The composition according to claim 67, wherein the at least one cell or the tissue comprises at least one selected from the group of: stem cells, satellite cells, muscle satellite cells, and progenitor cells.
